(19) United States
(12) Patent Application Publication
(10) Pub. No.: US 2023/0040182 A1
(43) Pub. Date: Feb. 9, 2023

(54) ARYL HETEROBICYCLIC COMPOUNDS AS Kv1.3 POTASSIUM SHAKER CHANNEL BLOCKERS

(71) Applicant: D. E. Shaw Research, LLC, New York, NY (US)

(72) Inventors: Fabrizio GIORDANETTO, New York, NY (US); Morten Østergaard JENSEN, Copenhagen (DK); Vishwanath JOGINI, Hyderabad (IN); Roger John SNOW, Danbury, CT (US)

(21) Appl. No.: 17/766,837

(22) PCT Filed: Oct. 6, 2020
(86) PCT No.: PCT/US2020/054348

§ 371 Date: Apr. 6, 2022

Related U.S. Application Data
(60) Provisional application No. 62/911,642, filed on Oct. 7, 2019.

(51) Int. Cl. C07D 471/04; C07D 471/14; C07D 487/04

(52) U.S. Cl. CPC C07D 471/04; C07D 471/14; C07D 487/04

(57) ABSTRACT

A compound of Formula (I), or a pharmaceutically acceptable salt thereof, is described, where the substituents are as defined herein. Pharmaceutical compositions including the same and method of using the same are also described.

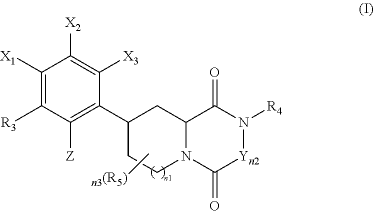

This application claims the benefit and priority of U.S. Provisional Application No. 62/911,642, filed Oct. 7, 2019, the entire contents of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Voltage-gated Kv1.3 potassium (K⁺) channels are expressed in lymphocytes (T and B lymphocytes), the central nervous system, and other tissues, and regulate a large number of physiological processes such as neurotransmitter release, heart rate, insulin secretion, and neuronal excitability. Kv1.3 channels can regulate membrane potential and thereby indirectly influence calcium signaling in human effector memory T cells. Effector memory T cells are mediators of several conditions, including multiple sclerosis, type I diabetes mellitus, psoriasis, spondylitis, parodontitis, and rheumatoid arthritis. Upon activation, effector-memory T cells increase expression of the Kv1.3 channel. Amongst human B cells, naive and early memory B cells express small numbers of Kv1.3 channels when they are quiescent. In contrast, class-switched memory B cells express high numbers of Kv1.3 channels. Furthermore, the Kv1.3 channel promotes the calcium homeostasis required for T-cell receptor-mediated cell activation, gene transcription, and proliferation (Panyi, G., et al., 2004, Trends Immunol., 565-569). Blockade of Kv1.3 channels in effector memory T cells suppresses activities like calcium signaling, cytokine production (interferon-gamma, interleukin 2), and cell proliferation.

Autoimmune disease is a family of disorders resulting from tissue damage caused by attack from the body's own immune system. Such diseases may affect a single organ, as in multiple sclerosis and type I diabetes mellitus, or may involve multiple organs, as in the case of rheumatoid arthritis and systemic lupus erythematosus. Treatment is generally palliative, with anti-inflammatory and immunosuppressive drugs, which can have severe side effects. A need for more effective therapies has led to a search for drugs that can selectively inhibit the function of effector memory T cells, known to be involved in the etiology of autoimmune diseases. These inhibitors are thought to be able to ameliorate autoimmune diseases symptoms without compromising the protective immune response. Effector memory T cells (TEMs) express high numbers of the Kv1.3 channel and depend on these channels for their function. In vivo, Kv1.3 channel blockers paralyze TEMs at the sites of inflammation and prevent their reactivation in inflamed tissues. Kv1.3 channel blockers do not affect the motility within lymph nodes of naive and central memory T cells. Suppressing the function of these cells by selectively blocking the Kv1.3 channel offers the potential for effective therapy of autoimmune diseases with minimal side effects.

Multiple sclerosis (MS) is caused by autoimmune damage to the central nervous system (CNS). Symptoms include muscle weakness and paralysis, which severely affect quality of life for patients. MS progresses rapidly and unpredictably and eventually leads to death. The Kv1.3 channel is also highly expressed in auto-reactive effector memory T cells from MS patients (Wulff H., et al., 2003, J. Clin. Invest., 1703-1713; Rus H., et al., 2005, PNAS, 11094-11099). Animal models of multiple sclerosis have been successfully treated using blockers of the Kv1.3 channel.

Compounds which are selective Kv1.3 channel blockers are thus potential therapeutic agents as immunosuppressants or immune system modulators. The Kv1.3 channel is also considered as a therapeutic target for the treatment of obesity and for enhancing peripheral insulin sensitivity in patients with type II diabetes mellitus. These compounds can also be utilized in the prevention of graft rejection and the treatment of immunological (e.g., autoimmune) and inflammatory disorders.

Tubulointerstitial fibrosis is a progressive connective tissue deposition on the kidney parenchyma, leading to renal function deterioration and is involved in the pathology of chronic kidney disease, chronic renal failure, nephritis, and inflammation in glomeruli, and is a common cause of end-stage renal failure. Overexpression of Kv1.3 channels in lymphocytes can promote their proliferation, leading to chronic inflammation and overstimulation of cellular immunity, which are involved in the underlying pathology of these renal diseases and are contributing factors in the progression of tubulointerstitial fibrosis. Inhibition of the lymphocyte Kv1.3 channel currents suppress proliferation of kidney lymphocytes and ameliorate the progression of renal fibrosis (Kazama I., et al., 2015, Mediators Inflamm., 1-12).

Kv1.3 channels also play a role in gastroenterological disorders including inflammatory bowel diseases (IBDs) such as ulcerative colitis (UC) and Crohn's disease. UC is a chronic IBD characterized by excessive T-cell infiltration and cytokine production. UC can impair quality of life and can lead to life-threatening complications. High levels of Kv1.3 channels in CD4 and CD8 positive T-cells in the inflamed mucosa of UC patients have been associated with production of pro-inflammatory compounds in active UC. Kv1.3 channels are thought to serve as a marker of disease activity and pharmacological blockade might constitute a novel immunosuppressive strategy in UC. Present treatment regimens for UC, including corticosteroids, salicylates, and anti-TNF-α reagents, are insufficient for many patients (Hansen L. K., et al., 2014, J. Crohns Colitis, 1378-1391). Crohn's disease is a type of IBD which may affect any part of the gastrointestinal tract. Crohn's disease is thought to be the result of intestinal inflammation due to a T-cell-driven process initiated by normally safe bacteria. Thus, Kv1.3 channel inhibition can be utilized in treating the Crohn's disease.

In addition to T cells, Kv1.3 channels are also expressed in microglia, where the channel is involved in inflammatory cytokine and nitric oxide production and in microglia-mediated neuronal killing. In humans, strong Kv1.3 channel expression has been found in microglia in the frontal cortex of patients with Alzheimer's disease and on CD68⁺ cells in multiple sclerosis brain lesions. It has been suggested that Kv1.3 channel blockers might be able to preferentially target detrimental proinflammatory microglia functions. Kv1.3 channels are expressed on activated microglia in infarcted rodent and human brain. Higher Kv1.3 channel current densities are observed in acutely isolated microglia from the infarcted hemisphere than in microglia isolated from the contralateral hemisphere of a mouse model of stroke (Chen Y. J., et al., 2017, Ann. Clin. Transl. Neurol., 147-161).

Expression of Kv1.3 channels is elevated in microglia of human Alzheimer's disease brains, suggesting that Kv1.3 channel is a pathologically relevant microglial target in Alzheimer's disease (Rangaraju S., et al., 2015, J. Alzheimers Dis., 797-808). Soluble AβO enhances microglial Kv1.3 channel activity. Kv1.3 channels are required for AβO-induced microglial pro-inflammatory activation and neurotoxicity. Kv1.3 channel expression/activity is upregulated in transgenic Alzheimer's disease animals and human Alzheimer's disease brains. Pharmacological targeting of microglial Kv1.3 channels can affect hippocampal synaptic plasticity and reduce amyloid deposition in APP/PS1 mice. Thus, the Kv1.3 channel may be a therapeutic target for Alzheimer's disease.

Kv1.3 channel blockers could be also useful for ameliorating pathology in cardiovascular disorders such as ischemic stroke, where activated microglia significantly contributes to the secondary expansion of the infarct.

Kv1.3 channel expression is associated with the control of proliferation in multiple cell types, apoptosis, and cell survival. These processes are crucial for cancer progression. In this context, Kv1.3 channels located in the inner mitochondrial membrane can interact with the apoptosis regulator Bax (Serrano-Albarras, A., et al., 2018, Expert Opin. Ther. Targets, 101-105). Thus, inhibitors of Kv1.3 channels may be used as anticancer agents.

A number of peptide toxins with multiple disulfide bonds from spiders, scorpions, and anemones are known to block Kv1.3 channels. A few selective, potent peptide inhibitors of the Kv1.3 channel have been developed. A synthetic derivative of stichodactyla toxin (shk) with an unnatural amino acid (shk-186) is the most advanced peptide toxin. Shk has demonstrated efficacy in preclinical models and is currently in a phase I clinical trial for treatment of psoriasis. Shk can suppress proliferation of TEM cells, resulting in improved condition in animal models of multiple sclerosis. Unfortunately, Shk also binds to the closely-related Kvi channel subtype found in CNS and heart. There is a need for Kv1.3 channel-selective inhibitors to avoid potential cardio- and neuro-toxicity. Additionally, small peptides like shk-186 are rapidly cleared from the body after administration, resulting in short circulating half-lives, frequent administration events. Thus, there is a need for the development of long-acting, selective Kv1.3 channel inhibitors for the treatment of chronic inflammatory diseases.

Thus, there remains a need for the development of novel Kv1.3 channel blockers as pharmaceutical agents.

### SUMMARY OF THE INVENTION

In one aspect, compounds useful as potassium channel blockers having a structure of Formula I

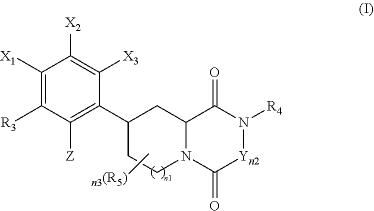

are described, where the various substituents are defined herein. The compounds of Formula I described herein can block Kv1.3 potassium (K⁺) channels and be used in the treatment of a variety of disease conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including as pharmaceutically active agents and methods for treating cancer, an immunological disorder, a CNS disorder, an inflammatory disorder, a gastroenterological disorder, a metabolic disorder, a cardiovascular disorder, a kidney disease, or a combination thereof.

In one aspect, a compound of Formula I or a pharmaceutically acceptable salt thereof is described,

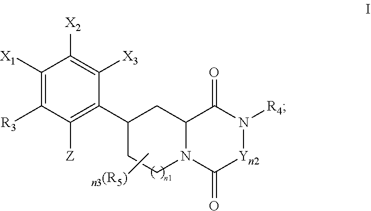

where

each occurrence of Y is independently C(R₂)₂ or NR₁;

Z is ORₐ;

X₁ is H, halogen, or alkyl;

X₂ is H, halogen, CN, alkyl, cycloalkyl, halogenated cycloalkyl, or halogenated alkyl;

X₃ is H, halogen, CN, alkyl, cycloalkyl, halogenated cycloalkyl, or halogenated alkyl;

or alternatively X₁ and X₂ and the carbon atoms they are connected to taken together form an optionally substituted 5- or 6-membered aryl;

or alternatively X₂ and X₃ and the carbon atoms they are connected to taken together form an optionally substituted 5- or 6-membered aryl;

each occurrence of R₁ is H, alkyl, cycloalkyl, heteroalkyl, or cycloheteroalkyl;

each occurrence of R₂ is H, alkyl, cycloalkyl, heteroalkyl, cycloheteroalkyl, or NRₐR_{b};

R₃ is H, alkyl, or halogen;

R₄ is H, alkyl, cycloalkyl, heteroalkyl, cycloheteroalkyl, or NRₐR_{b};

each occurrence of R₅ is H, halogen, OR₆, or alkyl, where each R₅ may be attached to any one of the carbon ring atoms of

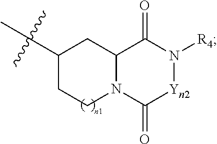

or alternatively R₁ and R₄ and the nitrogen atoms they are connected to taken together form an optionally substituted heterocycle;

or alternatively R₂ and R₄ and the carbon and nitrogen atoms they are connected to, respectively, taken together form an optionally substituted heterocycle;

each occurrence of Rₐ and R_{b} are independently H, alkyl, alkenyl, cycloalkyl, saturated heterocycle, aryl, or heteroaryl; or alternatively Rₐ and R_{b} together with the nitrogen atom that they are connected to form an optionally substituted heterocycle including the nitrogen atom and 0-3 additional heteroatoms each selected from the group consisting of N, O, and S;

the alkyl, cycloalkyl, heteroalkyl, cycloheteroalkyl, heterocycle, aryl, and heteroaryl in X₁, X₂, X₃, R₁, R₂, R₃, R₄, R₅, Rₐ, or R_{b}, where applicable, are each independently and optionally substituted by 1-4 substituents each independently selected from the group consisting of alkyl, cycloalkyl, halogenated alkyl, halogenated cycloalkyl, halogen, CN, OR₆, —(CH₂)₁₋₂OR₆, N(R₆)₂, (C═O)R₆, (C═O)N(R₆)₂, NR₆(C═O)R₆, and oxo where valence permits;

each occurrence of R₆ is independently H, alkyl, or heterocycle optionally substituted by alkyl; or alternatively two R₆ groups together with the nitrogen atom that they are connected to form a heterocycle optionally substituted by alkyl and including the nitrogen atom and 0-3 additional heteroatoms each selected from the group consisting of N, O, and S;

n₁ is an integer from 0-1;

n₂ is an integer from 0-2; and

n₃ is an integer from 0-2.

In any one of the embodiments described herein, the structural moiety

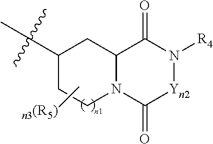

has the structure of

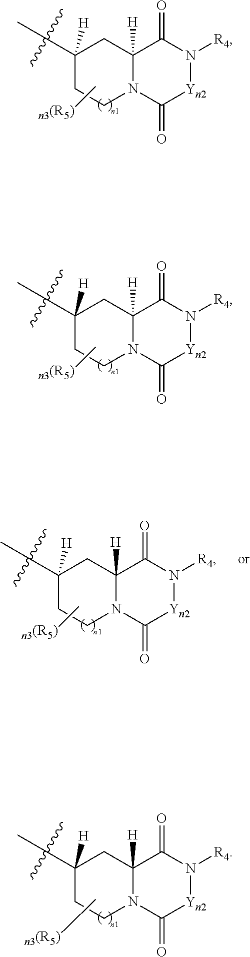

In any one of the embodiments described herein, the structural moiety

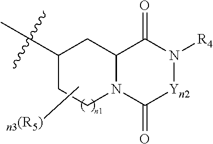

has the structure of

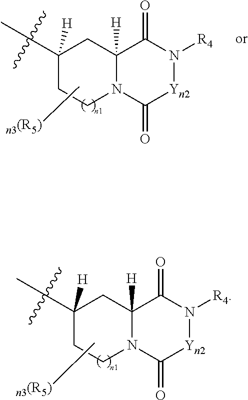

In any one of the embodiments described herein, the structural moiety

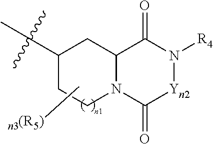

has the structure of

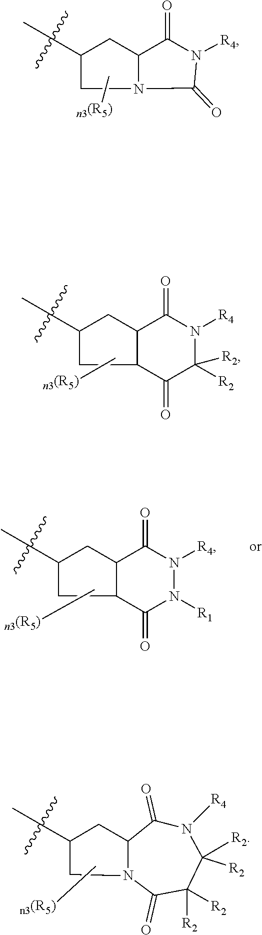

In any one of the embodiments described herein, the structural moiety

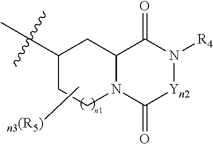

has the structure of

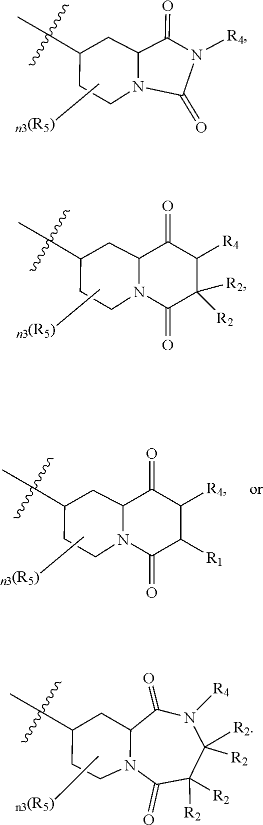

In any one of the embodiments described herein, the structural moiety

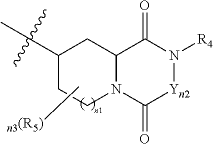

has the structure of

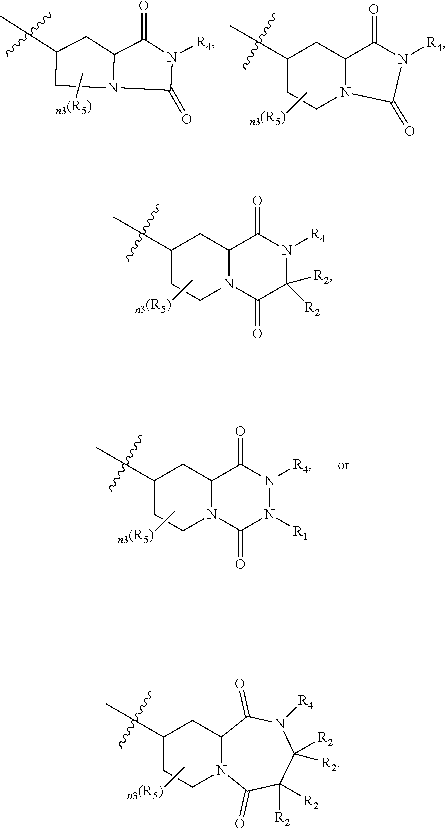

In any one of the embodiments described herein, the structural moiety

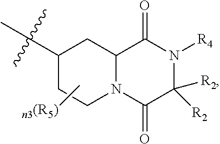

has the structure of

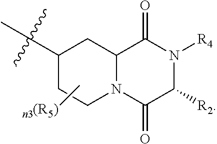

In any one of the embodiments described herein, at least one of R₁, R₂, and R₄ is H, alkyl, or cycloalkyl.

In any one of the embodiments described herein, at least one of R₁, R₂, and R₄ is H, Me, Et, n-Pr, iso-Pr, n-Bu, sec-Bu, or tert-Bu.

In any one of the embodiments described herein, at least one of R₂ and R₄ is alkyl or cycloalkyl each optionally substituted by one or more OR₆, N(R₆)₂, or —(CH₂)₁₋₂OR₆.

In any one of the embodiments described herein, at least one of R₂ and R₄ is

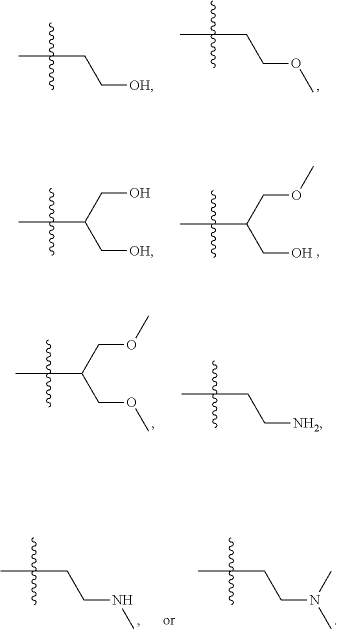

In any one of the embodiments described herein, at least one occurrence of R₂ is Me, Et,

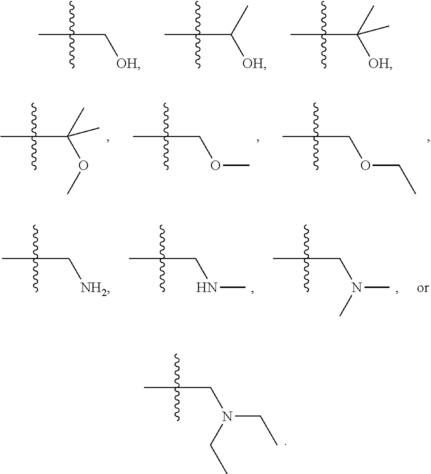

In any one of the embodiments described herein, at least one of R₂ and R₄ is

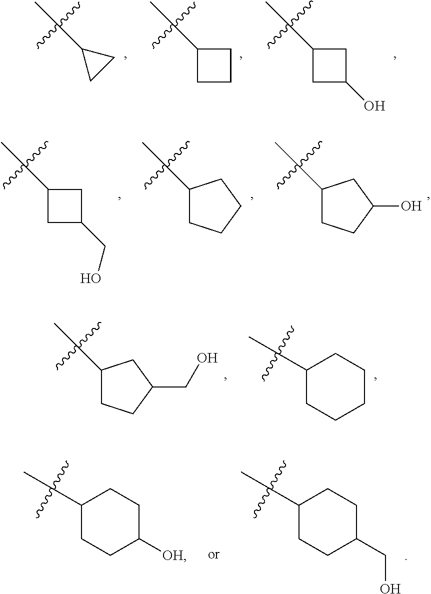

In any one of the embodiments described herein, at least one of R₁, R₂, and R₄ is heteroalkyl, or cycloheteroalkyl.

In any one of the embodiments described herein, at least one of R₂ and R₄ is NRₐR_{b}.

In any one of the embodiments described herein, Rₐ and R_{b} are each independently H, alkyl or cycloalkyl.

In any one of the embodiments described herein, at least one of R₂ and R₄ is NH₂, NHMe, or NHMe₂.

In any one of the embodiments described herein, at least one of R₂ and R₄ is cycloheteroalkyl optionally substituted by one or more alkyl.

In any one of the embodiments described herein, at least one of R₂ and R₄ is

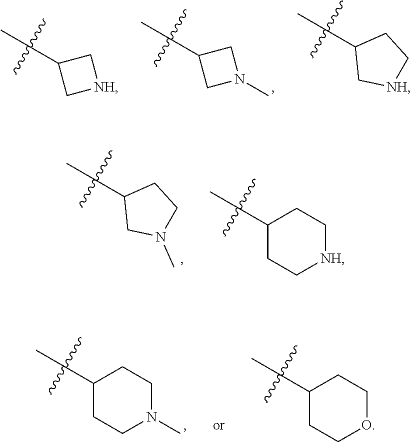

In any one of the embodiments described herein, R₁ and R₄ and the nitrogen atoms they are connected to taken together form an optionally substituted heterocycle; or where R₂ and R₄ and the carbon and nitrogen atoms they are connected to, respectively, taken together form an optionally substituted heterocycle.

In any one of the embodiments described herein, the structural moiety

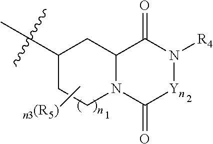

has the structure of

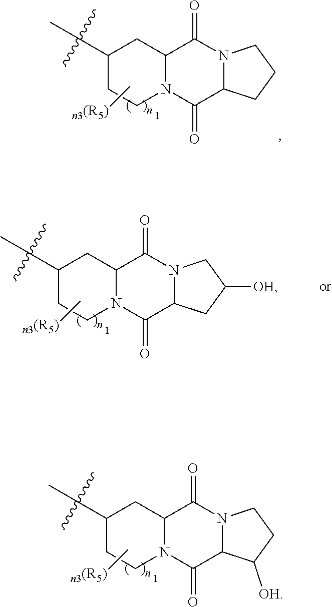

In any one of the embodiments described herein, the structural moiety

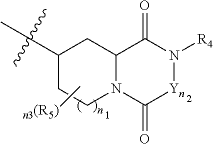

has the structure of

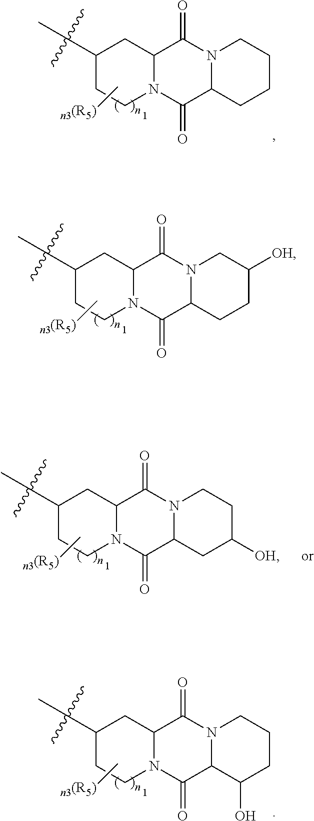

In any one of the embodiments described herein, at least one occurrence of R₅ is H or alkyl.

In any one of the embodiments described herein, at least one occurrence of R₅ is halogen or OH.

In any one of the embodiments described herein, n₃ is 0 or 1.

In any one of the embodiments described herein, Z is OH, OMe, OEt, OPr, or OBu.

In any one of the embodiments described herein, Z is OH or OMe.

In any one of the embodiments described herein, Z is OH.

In any one of the embodiments described herein, X₁ is H, halogen, or Me.

In any one of the embodiments described herein, X₁ is H or Cl.

In any one of the embodiments described herein, X₂ is H, halogen, fluorinated alkyl, or alkyl.

In any one of the embodiments described herein, X₂ is H, F, Cl, Br, Me, CF₂H, CF₂Cl, or CF₃.

In any one of the embodiments described herein, X₂ is H or Cl.

In any one of the embodiments described herein, X₃ is H, halogen, fluorinated alkyl, or alkyl.

In any one of the embodiments described herein, X₃ is H, F, Cl, Br, Me, CF₂H, CF₂Cl, or CF₃.

In any one of the embodiments described herein, X₃ is H or Cl.

In any one of the embodiments described herein, R₃ is H, Me, Et, Pr, F, Cl, or Br.

In any one of the embodiments described herein, R₃ is H.

In any one of the embodiments described herein, R₃ is Me, Et, or Pr.

In any one of the embodiments described herein, R₃ is F, Cl, or Br.

In any one of the embodiments described herein, the structural moiety

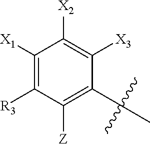

has the structure of

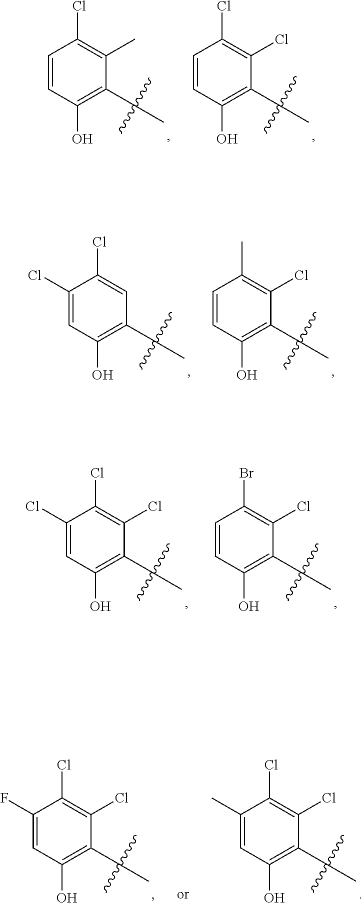

In any one of the embodiments described herein, the compound has a structure of Formula II′ or II:

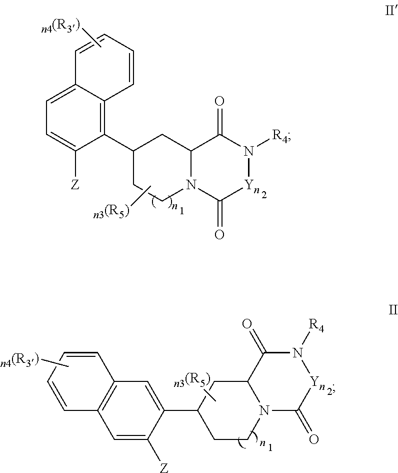

where R_{3′} is independently H, halogen, or alkyl; and

n₄ is an integer from 0-3.

In any one of the embodiments described herein, n₄ is 0, 1, or 2.

In any one of the embodiments described herein, n₄ is 0.

In any one of the embodiments described herein, R_{3′} is H or alkyl.

In any one of the embodiments described herein, R_{3′} is halogen.

In any one of the embodiments described herein, at least one occurrence of Rₐ or R_{b} is independently H, alkyl, cycloalkyl, saturated heterocycle, aryl, or heteroaryl.

In any one of the embodiments described herein, at least one occurrence of Rₐ or R_{b} is independently H, Me, Et, Pr, or a heterocycle selected from the group consisting of

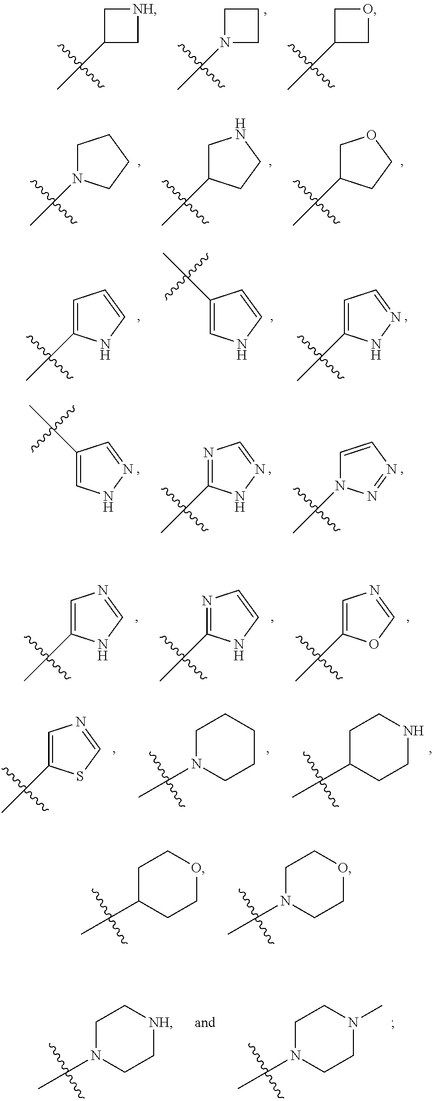

where the heterocycle is optionally substituted by alkyl, OH, oxo, or (C═O)C₁₋₄alkyl where valence permits.

In any one of the embodiments described herein, Rₐ and R_{b} together with the nitrogen atom that they are connected to form an optionally substituted heterocycle including the nitrogen atom and 0-3 additional heteroatoms each selected from the group consisting of N, O, and S.

In any one of the embodiments described herein, the compound is selected from the group consisting of compounds 1-70 as shown in Table 1.

In yet another aspect, a pharmaceutical composition is described, including at least one compound according to any one of the embodiments described herein or a pharmaceutically acceptable salt thereof and a pharmaceutically acceptable carrier or diluent.

In yet another aspect, a method of treating a condition in a mammalian species in need thereof is described, including administering to the mammalian species a therapeutically effective amount of at least one compound according to any one of the embodiments described herein or a pharmaceutically acceptable salt thereof, where the condition is selected from the group consisting of cancer, an immunological disorder, a central nervous system disorder, an inflammatory disorder, a gastroenterological disorder, a metabolic disorder, a cardiovascular disorder, and a kidney disease.

In any one of the embodiments described herein, the immunological disorder is transplant rejection or an autoimmune disease.

In any one of the embodiments described herein, the autoimmune disease is rheumatoid arthritis, multiple sclerosis, systemic lupus erythematosus, or type I diabetes mellitus.

In any one of the embodiments described herein, the central nervous system disorder is Alzheimer's disease.

In any one of the embodiments described herein, the inflammatory disorder is an inflammatory skin condition, arthritis, psoriasis, spondylitis, parodontitits, or an inflammatory neuropathy.

In any one of the embodiments described herein, the gastroenterological disorder is an inflammatory bowel disease.

In any one of the embodiments described herein, the metabolic disorder is obesity or type II diabetes mellitus.

In any one of the embodiments described herein, the cardiovascular disorder is an ischemic stroke.

In any one of the embodiments described herein, the kidney disease is chronic kidney disease, nephritis, or chronic renal failure.

In any one of the embodiments described herein, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

In any one of the embodiments described herein, the mammalian species is human.

In yet another aspect, a method of blocking Kv1.3 potassium channel in a mammalian species in need thereof is described, including administering to the mammalian species a therapeutically effective amount of at least one compound according to any one of the embodiments described herein or a pharmaceutically acceptable salt thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle, and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “heteroalkyl” refers to a straight- or branched-chain alkyl group preferably having from 2 to 12 carbons, more preferably 2 to 10 carbons in the chain, one or more of which has been replaced by a heteroatom selected from the group consisting of S, O, P and N. Exemplary heteroalkyls include, but are not limited to, alkyl ethers, secondary and tertiary alkyl amines, alkyl sulfides, and the like. The group may be a terminal group or a bridging group.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “heterocycloalkyl” or “cycloheteroalkyl” refers to a saturated or partially saturated monocyclic, bicyclic, or polycyclic ring containing at least one heteroatom selected from the group consisting of nitrogen, sulfur, and oxygen, preferably from 1 to 3 heteroatoms in at least one ring. Each ring is preferably from 3 to 10 membered, more preferably 4 to 7 membered. Examples of suitable heterocycloalkyl substituents include, but are not limited to, pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane. The group may be a terminal group or a bridging group.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings where two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to

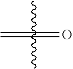 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure of

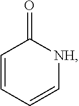

which also includes its tautomeric form of

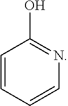

The term “alkylamino” refers to a group having the structure —NHR′, where R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, where R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a phenol or carboxylic acid, zwitterions (“inner salts”) may be formed and are included within the term “salt(s)” as used herein. Pharmaceutically-acceptable (i.e., non-toxic, physiologically-acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically-acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements, and hence may be preferred in some circumstances. Isotopically-labeled compounds can generally be prepared by carrying out the procedures disclosed in the Schemes and/or in the Examples below, by substituting a readily-available isotopically-labeled reagent for a non-isotopically-labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as Kv1.3 potassium channel blockers are described. Applicants have surprisingly discovered that the compounds disclosed herein exhibit potent Kv1.3 potassium channel-inhibiting properties. Additionally, Applicants have surprisingly discovered that the compounds disclosed herein selectively block the Kv1.3 potassium channel and do not block the hERG channel and, thus, have desirable cardiovascular safety profiles.

In one aspect, a compound of Formula I or a pharmaceutically acceptable salt thereof is described,

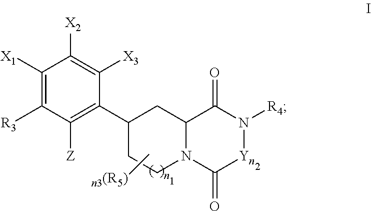

where

each occurrence of Y is independently C(R₂)₂ or NR₁;

Z is ORₐ;

X₁ is H, halogen, or alkyl;

X₂ is H, halogen, CN, alkyl, cycloalkyl, halogenated cycloalkyl, or halogenated alkyl;

X₃ is H, halogen, CN, alkyl, cycloalkyl, halogenated cycloalkyl, or halogenated alkyl;

or alternatively X₁ and X₂ and the carbon atoms they are connected to taken together form an optionally substituted 5- or 6-membered aryl;

or alternatively X₂ and X₃ and the carbon atoms they are connected to taken together form an optionally substituted 5- or 6-membered aryl;

each occurrence of R₁ is H, alkyl, cycloalkyl, heteroalkyl, or cycloheteroalkyl;

each occurrence of R₂ is H, alkyl, cycloalkyl, heteroalkyl, cycloheteroalkyl, or NRₐR_{b};

R₃ is H, alkyl, or halogen;

R₄ is H, alkyl, cycloalkyl, heteroalkyl, cycloheteroalkyl, or NRₐR_{b};

each occurrence of R₅ is H, halogen, OR₆, or alkyl, where each R₅ may be attached to any one of the carbon ring atoms of

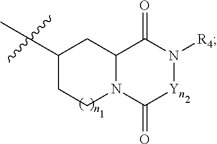

or alternatively R₁ and R₄ and the nitrogen atoms they are connected to taken together form an optionally substituted heterocycle;

or alternatively R₂ and R₄ and the carbon and nitrogen atoms they are connected to, respectively, taken together form an optionally substituted heterocycle;

each occurrence of Rₐ and R_{b} are independently H, alkyl, alkenyl, cycloalkyl, saturated heterocycle, aryl, or heteroaryl; or alternatively Rₐ and R_{b} together with the nitrogen atom that they are connected to form an optionally substituted heterocycle including the nitrogen atom and 0-3 additional heteroatoms each selected from the group consisting of N, O, and S;

the alkyl, cycloalkyl, heteroalkyl, cycloheteroalkyl, heterocycle, aryl, and heteroaryl in X₁, X₂, X₃, R₁, R₂, R₃, R₄, R₅, Rₐ, or R_{b}, where applicable, are each independently and optionally substituted by 1-4 substituents each independently selected from the group consisting of alkyl, cycloalkyl, halogenated alkyl, halogenated cycloalkyl, halogen, CN, OR₆, —(CH₂)₁₋₂OR₆, N(R₆)₂, (C═O)R₆, (C═O)N(R₆)₂, NR₆(C═O)R₆, and oxo where valence permits;

each occurrence of R₆ is independently H, alkyl, or heterocycle optionally substituted by alkyl; or alternatively two R₆ groups together with the nitrogen atom that they are connected to form a heterocycle optionally substituted by alkyl and including the nitrogen atom and 0-3 additional heteroatoms each selected from the group consisting of N, O, and S;

n₁ is an integer from 0-1;

n₂ is an integer from 0-2; and

n₃ is an integer from 0-2.

In some embodiments, the structural moiety

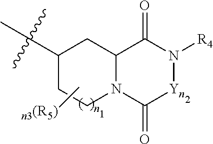

has the structure of

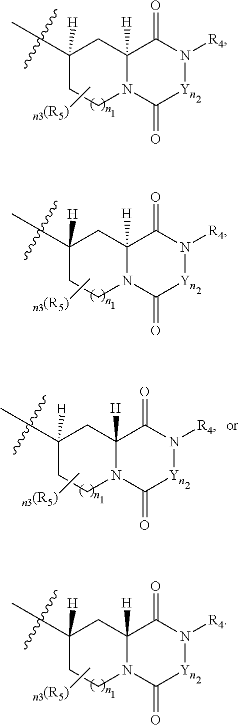

In some embodiments, the structural moiety

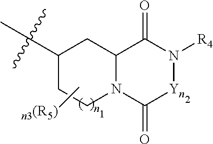

has the structure of

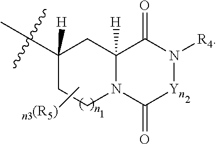

In some embodiments, the structural moiety

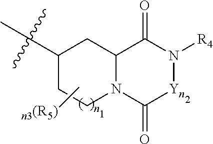

has the structure of

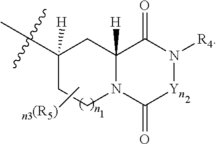

In some embodiments, the structural moiety

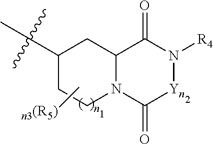

has the structure of

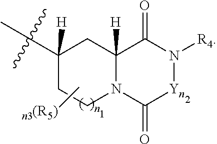

In some embodiments, the structural moiety

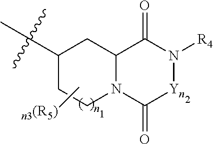

has the structure of

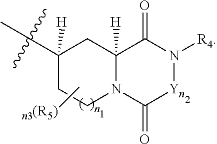

In some embodiments, n₁ is 1. In some embodiments, n₁ is 0. In some embodiments, n₂ is an integer from 0-2. In some embodiments, n₂ is an integer from 1-2. In some embodiments, n₂ is 0. In some embodiments, n₂ is 1 or 2. In some embodiments, n₂ is 2. In some embodiments, n₂ is 1.

In some embodiments, at least one occurrence of Y is C(R₂)₂. In other embodiments, at least one occurrence of Y is NR₁. In some embodiments, Yₙ₂ is —C(R₂)₂. In other embodiments, Yₙ₂ is —NR₁—. In still other embodiments, Yₙ₂ is —C(R₂)₂—C(R₂)₂—. In still other embodiments, the structural moiety —(C═O)—Yₙ₂— is —(C═O)—C(R₂)₂—NR₁—. In still other embodiments, the structural moiety —(C═O)—Yₙ₂— is —(C═O)—NR₁—C(R₂)₂—.

In some embodiments, the structural moiety

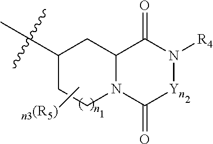

has the structure of

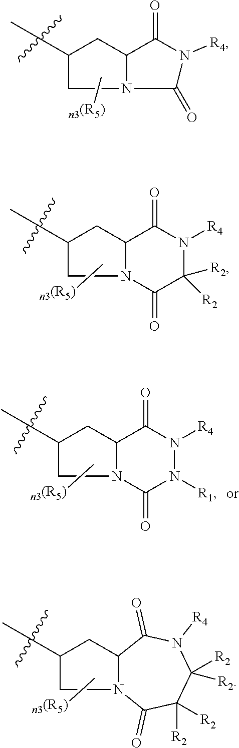

In some embodiments, the structural moiety

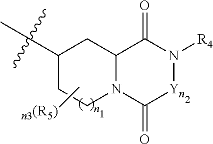

has the structure of

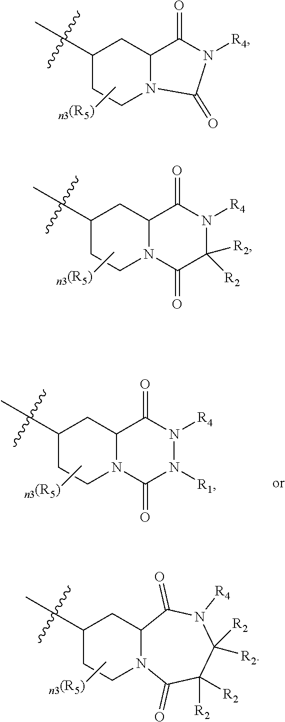

In some specific embodiments, the structural moiety

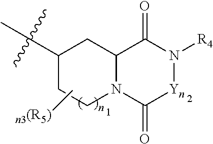

has the structure of

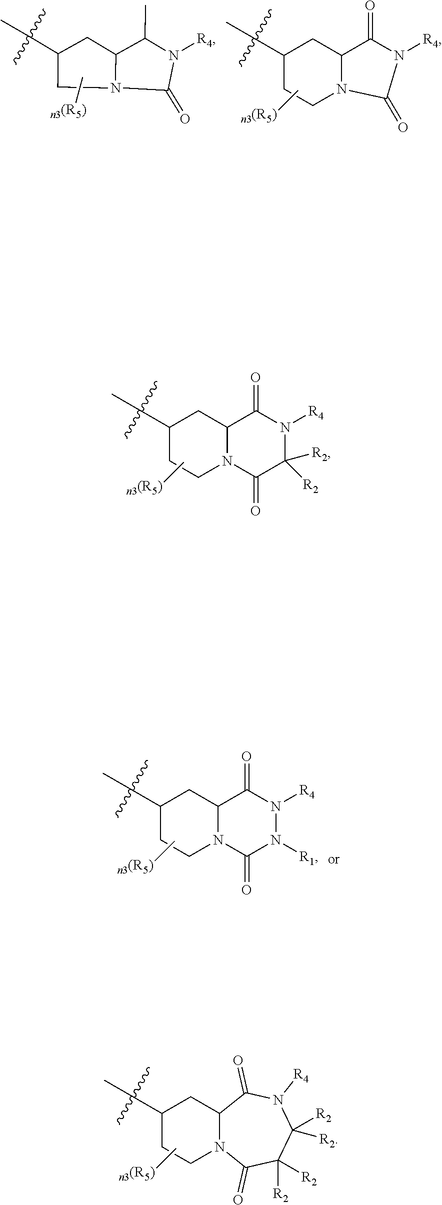

In some specific embodiments, the structural moiety

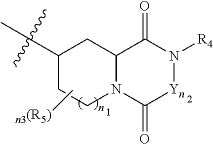

has the structure of

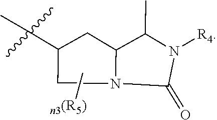

In some specific embodiments, the structural moiety

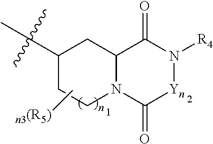

has the structure of

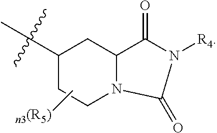

In some specific embodiments, the structural moiety

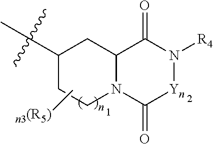

has the structure of

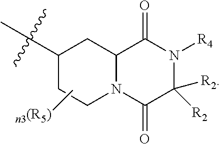

In some specific embodiments, the structural moiety

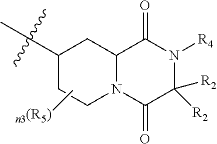

has the structure of

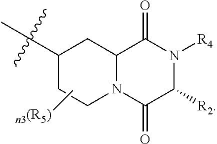

In some embodiments, R₁ is H, alkyl, or cycloalkyl. In other embodiments, R₁ is heteroalkyl, or cycloheteroalkyl.

In some embodiments, at least one occurrence of R₂ is H, alkyl, or cycloalkyl. In some specific embodiments, at least one occurrence of R₂ is H, Me, Et, n-Pr, iso-Pr, n-Bu, sec-Bu, or tert-Bu. In other specific embodiments, at least one occurrence of R₂ is alkyl or cycloalkyl each optionally substituted by one or more OR₆, N(R₆)₂, or —(CH₂)₁₋₂OR₆. In some specific embodiments, at least one occurrence of R₂ is

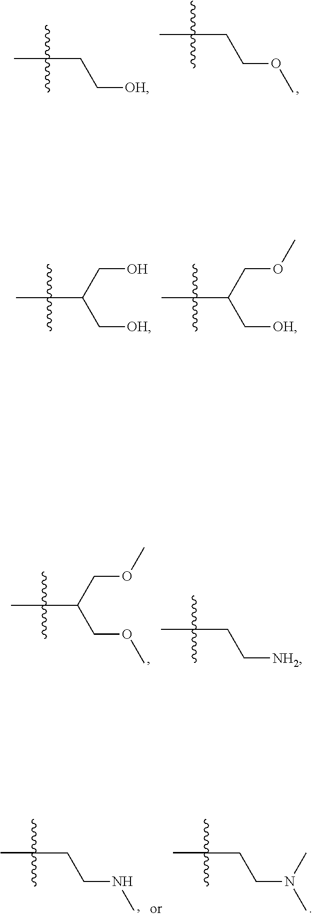

In some embodiments, at least one occurrence of R₂ is Me, Et,

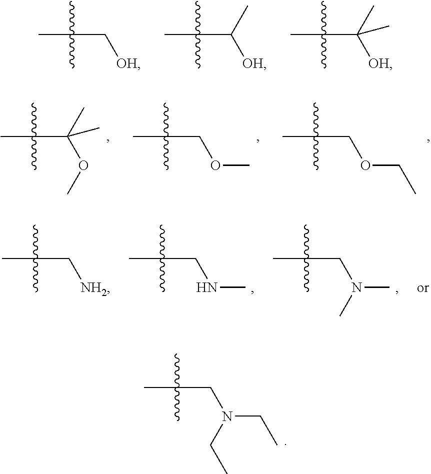

In some embodiments, at least one occurrence of R₂ is

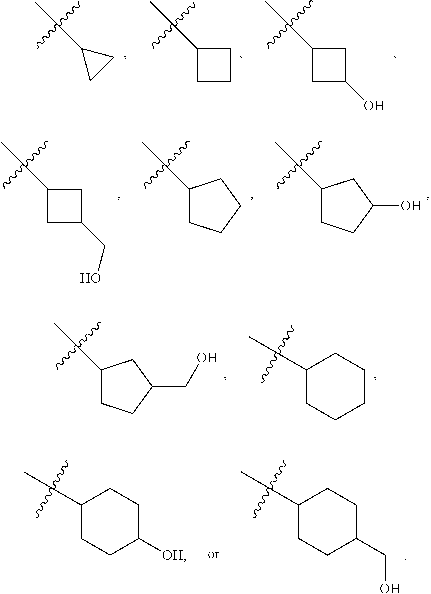

some specific embodiments, at least one occurrence of R₂ is heteroalkyl, cycloheteroalkyl, or NRₐR_{b}. In some specific embodiments, at least one occurrence of R₂ is NRₐR_{b}, such as NH₂, NHMe, or NHMe₂. In some specific embodiments, R₂ is NRₐR_{b} and Rₐ is H and R_{b} is alkyl or cycloalkyl. In some specific embodiments, R₂ is NRₐR_{b} and each of Rₐ and R_{b} is alkyl or cycloalkyl. In other embodiments, at least one occurrence of R₂ is cycloheteroalkyl optionally substituted by one or more alkyl. In some specific embodiments, R₂ is

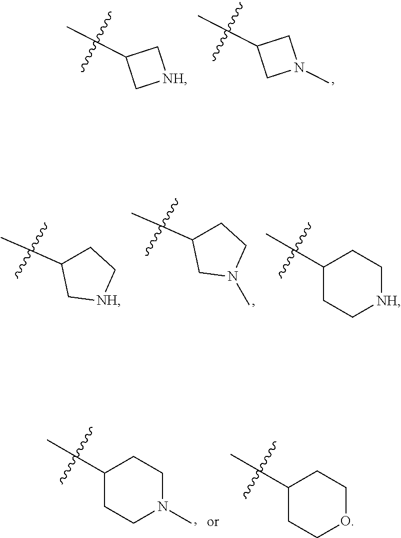

In some embodiments, R₂ is heteroalkyl. In some specific embodiments, R₂ is alkyl ethers, secondary and tertiary alkyl amines, or alkyl sulfides, such as —CH₂—CH₂—OMe, —CH₂—CH₂-OEt, —CH₂—CH₂—OPr, —CH₂—CH₂—SMe, —CH₂—CH₂-SEt, —CH₂—CH₂—SPr, —CH₂—CH₂—NHMe, —CH₂—CH₂—NMe₂, —CH₂—CH₂-NEtMe, or —CH₂—CH₂-NEt₂. In some embodiments, R₂ is cycloheteroalkyl. Non-limiting examples of cycloheteroalkyl include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane.

In some embodiments, R₄ is H, alkyl, or cycloalkyl. In some specific embodiments, R₄ is H, Me, Et, n-Pr, iso-Pr, n-Bu, sec-Bu, or tert-Bu. In other specific embodiments, R₄ is alkyl or cycloalkyl each optionally substituted by one or more OR₆, N(R₆)₂, or —(CH₂)₁₋₂OR₆. In some specific embodiments, R₄ is

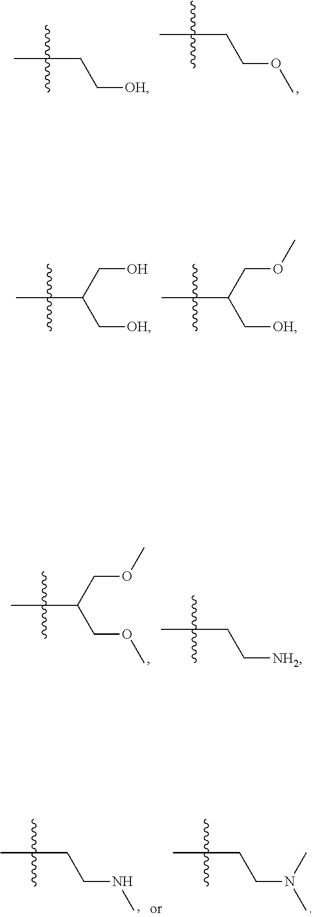

In some embodiments, R₄ is

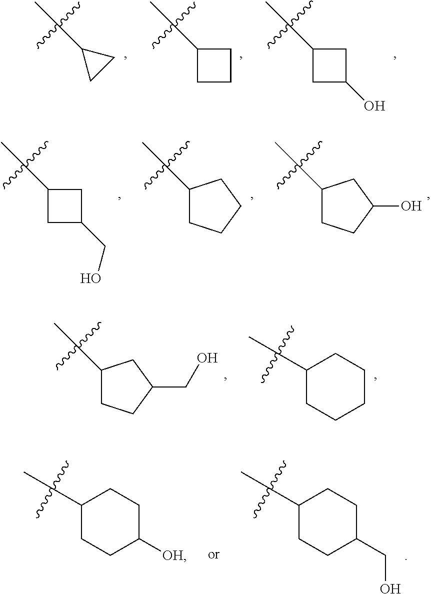

In some specific embodiments, R₄ is heteroalkyl, cycloheteroalkyl, or NRₐR_{b}. In some specific embodiments, R₄ is NRₐR_{b}, such as NH₂, NHMe, or NHMe₂. In some specific embodiments, R₄ is NRₐR_{b} and Rₐ is H and R_{b} is alkyl or cycloalkyl. In some specific embodiments, R₄ is NRₐR_{b} and each of Rₐ and R_{b} is alkyl or cycloalkyl. In other embodiments, R₄ is cycloheteroalkyl optionally substituted by one or more alkyl. In some specific embodiments, R₄ is

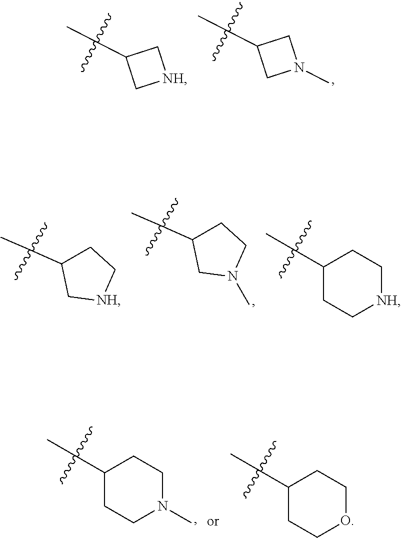

In some specific embodiments, R₄ is alkyl ethers, secondary and tertiary alkyl amines, or alkyl sulfides, such as —CH₂—CH₂—OMe, —CH₂—CH₂-OEt, —CH₂—CH₂—OPr, —CH₂—CH₂—SMe, —CH₂—CH₂—SEt, —CH₂—CH₂—SPr, —CH₂—CH₂—NHMe, —CH₂—CH₂—NMe₂, —CH₂—CH₂-NEtMe, or —CH₂—CH₂-NEt₂. In some embodiments, R₄ is cycloheteroalkyl. Non-limiting examples of cycloheteroalkyl include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane.

In other embodiments, R₁ and R₄ and the nitrogen atoms they are connected to taken together form an optionally substituted heterocycle. In still other embodiments, R₂ and R₄ and the carbon and nitrogen atoms they are connected to, respectively, taken together form an optionally substituted heterocycle.

In some specific embodiments, the structural moiety

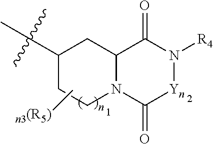

structure of

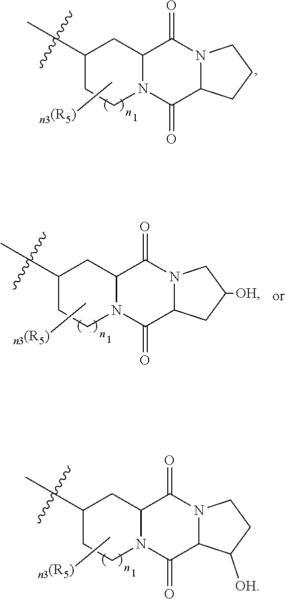

In some specific embodiments, the structural moiety

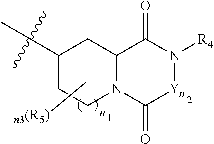

has the structure of

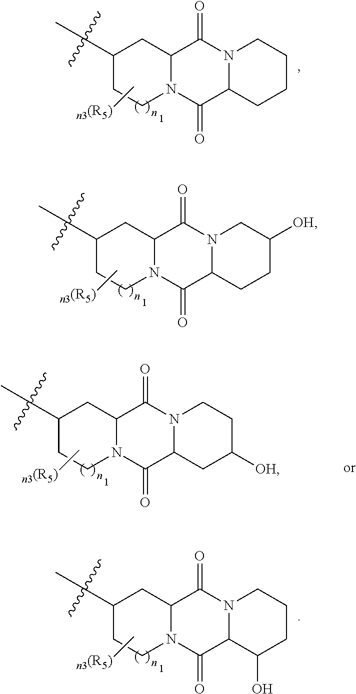

In some specific embodiments, the structural moiety

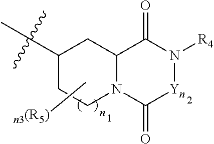

has the structure of

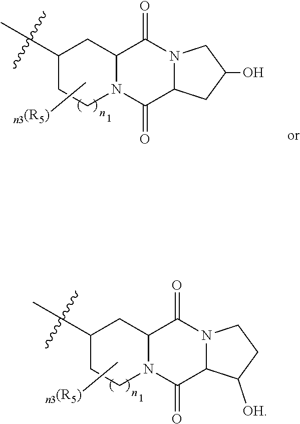

In some embodiments, at least one occurrence of R₅ is H or alkyl. Non-limiting examples of alkyl include Me, Et, propyl, isopropyl, n-butyl, iso-butyl, or sec-butyl. In other embodiments, R₅ is OR₆ or halogen. In some specific embodiments, R₅ is halogen. In some specific embodiments, R₅ is OR₆. In some specific embodiments, R₅ is OH. In some embodiments, n₃ is 2. In some embodiments, n₃ is 1. In some embodiments, n₃ is 0.

In some embodiments, R₆ is H or alkyl. In other embodiments, R₆ is optionally substituted heterocycle. In still other embodiments, the two R₆ groups together with the nitrogen atom that they are connected to form an optionally substituted heterocycle including the nitrogen atom and 0-3 additional heteroatoms each selected from the group consisting of N, O, and S.

In some embodiments, Z is ORₐ. In some embodiments, Z is OH, OMe, OEt, OPr, or OBu. In some embodiments, Z is OH.

In some embodiments, X₁ is H, halogen, or alkyl. In any one of the embodiments described herein, X₁ may be H or halogen. In some embodiments, X₁ is H or alkyl. In other embodiments, X₁ is alkyl. In other embodiments, X₁ is H. In some embodiments, X₁ is H, F, Cl, Br, or Me. In some embodiments, X₁ is H, F, or Cl. In some embodiments, X₁ is F or Cl. In some embodiments, X₁ is H or Cl. In some embodiments, X₁ is F. In some embodiments, X₁ is Cl. In some embodiments, X₁ is H.

In some embodiments, X₂ is H, halogen, CN, alkyl, halogenated alkyl, cycloalkyl, or halogenated cycloalkyl. In any one of the embodiments described herein, X₂ may be H, halogen, fluorinated alkyl, or alkyl. In some embodiments, X₂ is H or halogen. In other embodiments, X₂ is fluorinated alkyl or alkyl. In other embodiments, X₂ is cycloalkyl. In some embodiments, X₂ is H, F, Cl, Br, Me, CF₂H, CF₂Cl, or CF₃. In some embodiments, X₂ is H, F, or Cl. In some embodiments, X₂ is F or Cl. In some embodiments, X₂ is H or Cl. In some embodiments, X₂ is F. In some embodiments, X₂ is CF₃. In some embodiments, X₂ is CF₂Cl. In some embodiments, X₂ is Cl.

In some embodiments, X₃ is H, halogen, CN, alkyl, halogenated alkyl, cycloalkyl, or halogenated cycloalkyl. In any one of the embodiments described herein, X₃ may be H, halogen, fluorinated alkyl, or alkyl. In some embodiments, X₃ is H or halogen. In other embodiments, X₃ is fluorinated alkyl or alkyl. In other embodiments, X₃ is cycloalkyl. In some embodiments, X₃ is H, F, Cl, Br, Me, CF₂H, CF₂Cl, or CF₃. In some embodiments, X₃ is H, F, or Cl. In some embodiments, X₃ is F or Cl. In some embodiments, X₃ is H or Cl. In some embodiments, X₃ is F. In some embodiments, X₃ is CF₃. In some embodiments, X₃ is CF₂Cl. In some embodiments, X₃ is Cl.

In some embodiments, the structural moiety

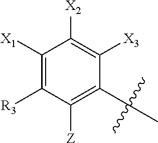

has the structure of

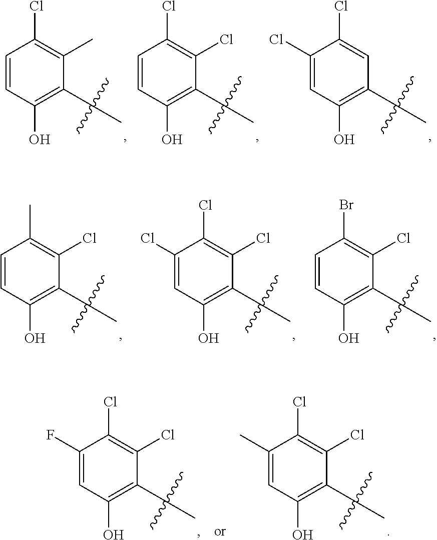

In any one of the embodiments described herein, R₃ is H, alkyl, or halogen. In some embodiments, R₃ is H or halogen. In some embodiments, R₃ is H, F, Cl, or Br. Non-limiting examples of alkyl include Me, Et, propyl, isopropyl, n-butyl, iso-butyl, and sec-butyl.

In some embodiments, the compound of Formula I has a structure of Formula II′ or II,

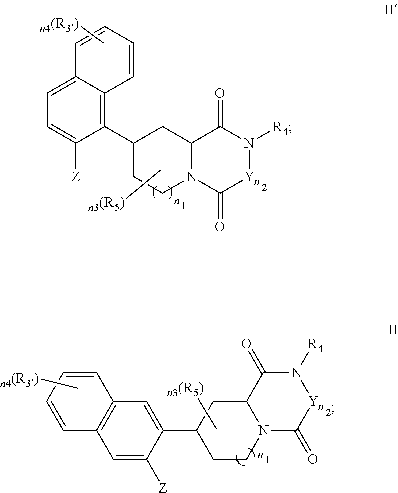

where each occurrence of R_{3′} is independently H, halogen, or alkyl; and n₄ is an integer from 0-3 and other substituents are as defined herein.

In some embodiments, Z is ORₐ. In some embodiments, Z is OH, OMe, OEt, OPr, or OBu. In some embodiments, Z is OH.

In some embodiments, n₄ is an integer from 0-3. In some embodiments, n₄ is an integer from 1-3. In some embodiments, n₄ is 0. In some embodiments, n₄ is 1 or 2. In some embodiments, n₄ is 1. In some embodiments, R_{3′} is H or alkyl. In some embodiments, R_{3′} is H. In some embodiments, R_{3′} is alkyl. In some embodiments, R_{3′} is halogen.

In any one of the embodiments described herein, at least one occurrence of Rₐ or R_{b} is independently H, alkyl, cycloalkyl, saturated heterocycle, aryl, or heteroaryl. In some embodiments, at least one occurrence of Rₐ or R_{b} is independently H, Me, Et, Pr, or Bu. In some embodiments, at least one occurrence of Rₐ or R_{b} is independently a heterocycle selected from the group consisting of

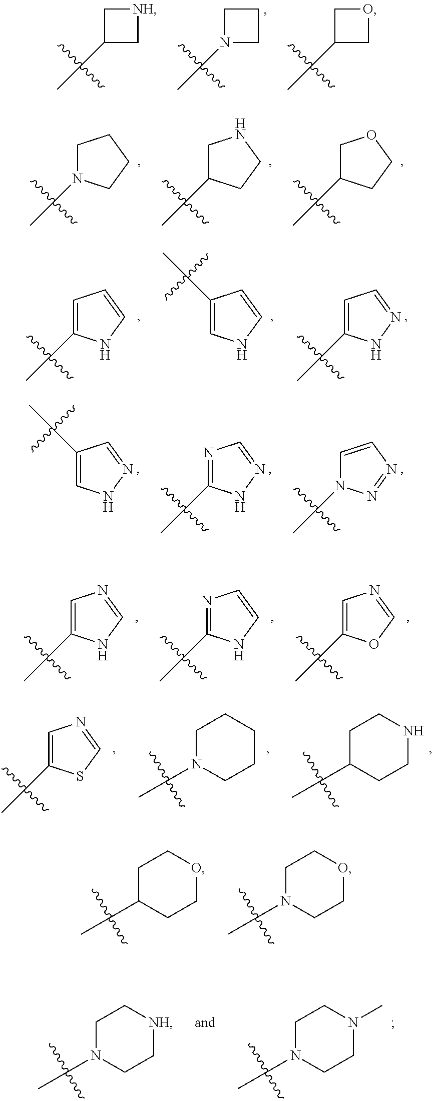

where the heterocycle is optionally substituted by alkyl, OH, oxo, or (C═O)C₁₋₄alkyl where valence permits.

In some embodiments, Rₐ and R_{b} together with the nitrogen atom that they are connected to form an optionally substituted heterocycle including the nitrogen atom and 0-3 additional heteroatoms each selected from the group consisting of N, O, and S.

In some embodiments, the compound of Formula I is selected from the group consisting of compounds 1-70 as shown in Table 1 below.

### Abbreviations

ACN Acetonitrile
Boc Tert-butyloxycarbonyl
CDI Carbonyldiimidazole
DCM Dichloromethane
DIPA Diisopropylamine
DIPEA N,N-diisopropylethylamine
DMAP 4-Dimethylaminopyridine
DMF Dimethyl formamide
EA Ethyl acetate
EDCI 1-Ethyl-3-(3-dimethylaminopropyl)carbodiimide
HATU N-[(dimethylamino)(3H-1,2,3-triazolo(4,4-b)pyridin-3-yloxy)methylene]-N-methylmethaneaminium hexafluorophosphate
HBTU 2-(1H-Benzotriazole-1-yl)-1,1,3,3-tetramethyluronium hexafluorophosphate
HOBT 1-Hydroxybenzotriazole
IPA Isopropanol
PE Petroleum ether
TEA Triethylamine
TFA Trifluoroacetic acid
THF Tetrahydrofuran

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations, but not limitations of the preparation of some of the starting materials and compounds disclosed herein.

Schemes 1-6 below describe synthetic routes which may be used for the synthesis of compounds of the present invention, e.g., compounds having a structure of Formula I or a precursor thereof. Various modifications to these methods may be envisioned by those skilled in the art to achieve similar results to that of the inventions given below. In the embodiments below, the synthetic route is described using compounds having the structure of Formula I or a precursor thereof as examples. The general synthetic routes described in Schemes 1-6 and examples described in the Example section below illustrate methods used for the preparation of the compounds described herein.

Compounds I-1a and I-2 as shown immediately below in Scheme 1 can be prepared by any method known in the art and/or are commercially available. As shown in Scheme 1, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH or an amine group. Other substituents are defined herein. As shown in Scheme 1, in some embodiments, the compounds disclosure herein can be synthesized from a suitable substituted bromo or iodo benzene I-1a, which is converted to the corresponding boronic acid I-1b by metalation with for example n-butyl lithium and reaction with a trialkyl borate such as trimethyl borate. Ketoester I-2 is reacted with a base such as lithium hexamethyldisilazide and N-phenyl triflimide to form the enol trifluoromethanesulfonate I-3. Coupling of I-3 with boronic acid I-1b in the presence of a catalyst such as 1,1′-bis(diphenylphosphino) ferrocene dichloropalladium(II) (Pd(dppf)Cl₂) gives cyclic amine I-4. Hydrogenation of I-4 over a catalyst such as platinum oxide yields the saturated cyclic amine ester I-5a. The protecting groups in compound I-5a can then be removed to give a compound of Formula I, and such compound with the free phenol OH and/or free nitrogen groups can optionally be further converted to other compounds of Formula I using methods known in the art.

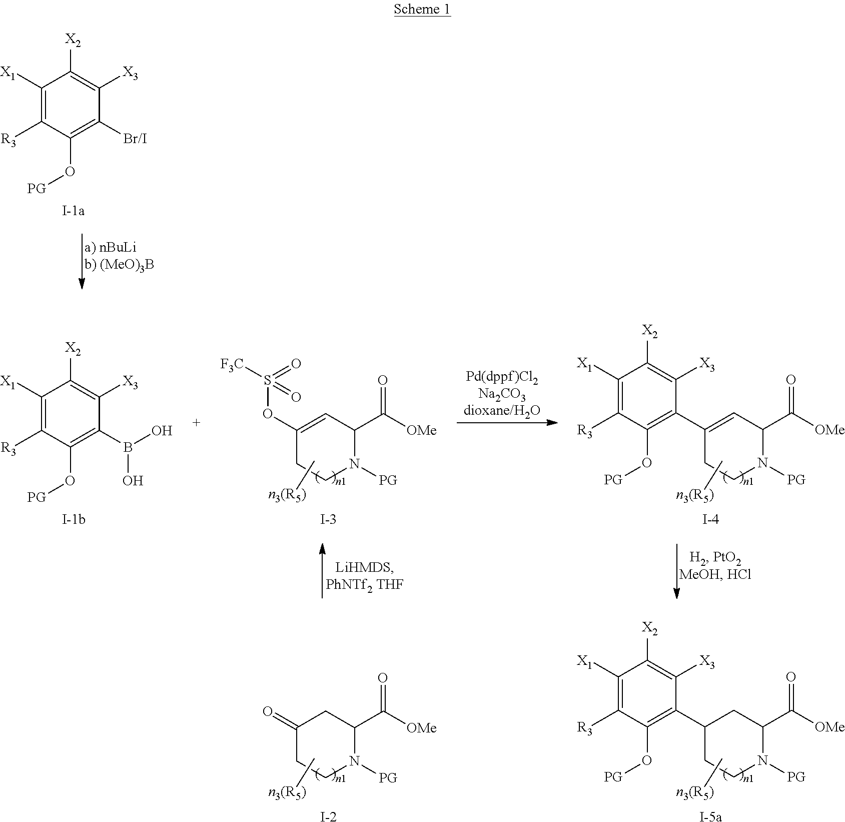

Compounds I-1a and I-6 as shown immediately below in Scheme 2 can be prepared by any method known in the art and/or are commercially available. As shown in Scheme 2, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. As shown in Scheme 2, in some embodiments, the compounds disclosure herein where n₁=1 can be prepared by an alternative route. The iodo or bromo benzene I-1a is coupled with a pyridine boronate ester I-6 in the presence of a palladium catalyst such as Pd(dppf)Cl₂ to form the 4-aryl pyridine I-7. Hydrogenation of I-7 over a catalyst such as platinum oxide provides the 4-aryl piperidine I-5b. The protecting groups in compound I-5b can then be removed to give a compound of Formula I, and such compound with the free phenol OH and/or free nitrogen groups can optionally be further converted to other compounds of Formula I using methods known in the art.

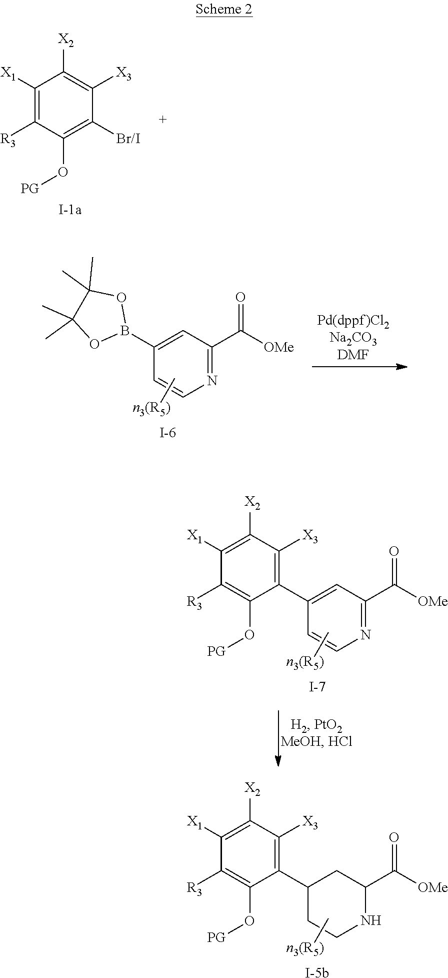

As shown in Scheme 3, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. As shown immediately below in Scheme 3, compounds as disclosed herein where R₄ is H or lower alkyl can be obtained from piperidine esters I-5b, or I-5c which can be obtained from I-5a by selectively removing the protecting group on the nitrogen. The cyclic amine ester I-5b or I-5c is coupled with a suitably protected amino acid using a coupling reagent such as EDC/HOBt, HBTU or HATU to form amide I-8. One example of a suitable amine protecting group on the nitrogen is t-butyl oxycarbonyl (boc). Removal of the amine protecting group using TFA followed by heating with a base such as triethylamine in a solvent such as toluene results in cyclization to the diketopiperazine I-9a. Removal of the phenol protecting group yields I-10a.

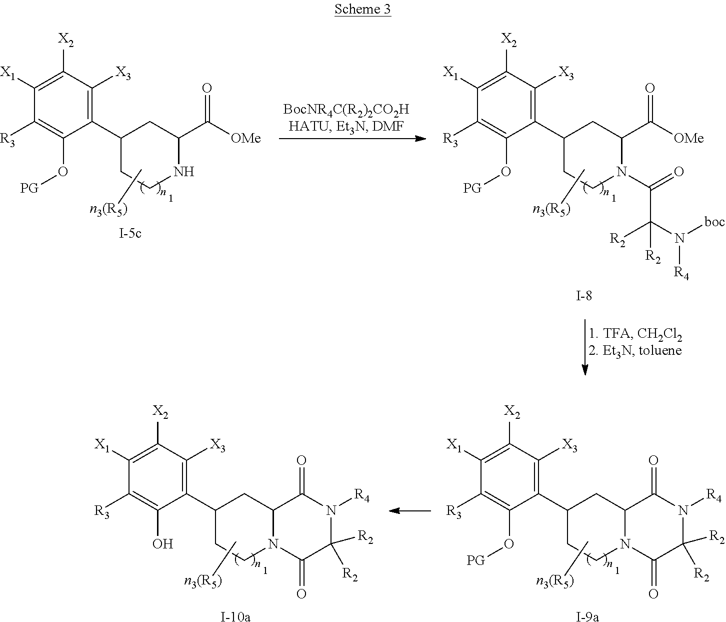

As shown in Scheme 4, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. A related method applicable for more complex R₄ groups is shown immediately below in Scheme 4. Reaction of amino ester I-5c with chloroacetyl chloride and a base such as triethylamine gives the chloro acetamide I-11. Treatment of I-11 with an amine R₄NH₂ and as base such as triethylamine and heating in a solvent such as ethanol yield the N-substituted diketopiperazine I-9b which is converted to I-10b by removal of the phenol protecting group.

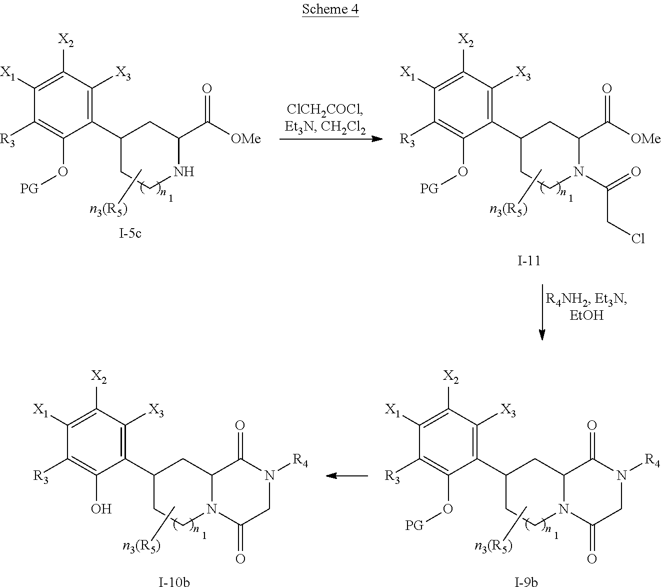

As shown in Scheme 5, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. Compounds as disclosed herein where Y is either absent or is nitrogen can be synthesized by the methods shown immediately below in Scheme 5. As shown in Scheme 5, for compounds as disclosed herein where Y is absent, reaction of amino ester I-5c with an amine R₄NH₂ by heating in methanol provides amide I-12. Treatment of amide I-12 with carbonyl diimidazole (CDI) in DMF causes cyclization to the imidazolinedione I-13. For compounds as disclosed herein where Y is nitrogen, reaction of the boc-protected amino ester I-5d with hydrazine hydrate gives the triazolidinedione I-14 directly. The protecting groups in compounds I-13 and I-14 can then be removed to give compounds of Formula I, and such compounds with the free phenol OH group can optionally be further converted to other compounds of Formula I using methods known in the art.

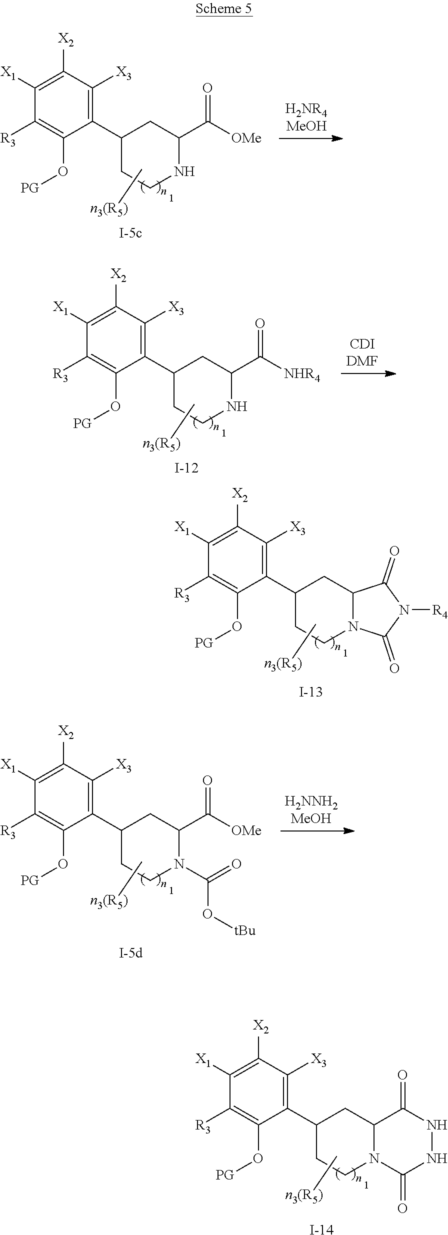

As shown in Scheme 6, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. A stereocontrolled synthesis of intermediate 5d to result in chiral intermediate 5e is shown immediately below in Scheme 6. As shown in Scheme 6, the enantiomerically pure piperidone I-15 can be synthesized from protected L-aspartic acid and Meldrum's acid by the method of Org. Syn., 2008, 85, 147, and then converted to the enol triflate I-16 by treatment with trifluoromethansulfonic anhydride and base according to the procedures described in Syn. Lett., 2009, 71-74. The enol triflate I-16 is coupled with boronic acid I-1b using a palladium catalyst such as Pd(dppf)Cl₂ to yield I-17. Hydrogenation of I-17 over a catalyst such as platinum oxide gives piperidinone I-18 predominantly as the 2S,4S enantiomer, and reduction of the amide using borane methyl sulfide complex provides enantiomerically pure I-5e, which can be used in the syntheses outlined in Schemes 3, 4 and 5.

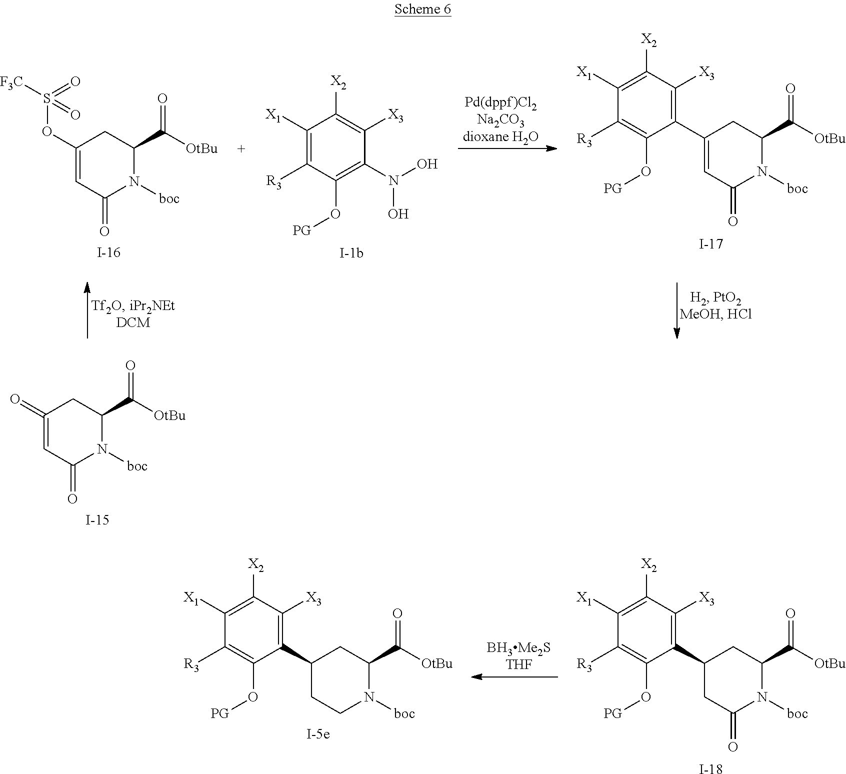

The reactions described above in Schemes 1-6 can be carried out in a suitable solvent. Suitable solvents include, but are not limited to, acetonitrile, methanol, ethanol, dichloromethane, DMF, THF, MTBE, or toluene. The reactions described in Schemes 1-6 may be conducted under inert atmosphere, e.g., under nitrogen or argon, or the reaction may be carried out in a sealed tube. The reaction mixture may be heated in a microwave or heated to an elevated temperature. Suitable elevated temperatures include, but are not limited to, 40, 50, 60, 80, 90, 100, 110, 120° C. or higher or the refluxing/boiling temperature of the solvent used. The reaction mixture may alternatively be cooled in a cold bath at a temperature lower than room temperature, e.g., 0, −10, −20, −30, −40, −50, −78, or −90° C. The reaction may be worked up by removing the solvent or partitioning of the organic solvent phase with one or more aqueous phases, each optionally containing NaCl, NaHCO₃, or NH₄Cl. The solvent in the organic phase can be removed by reduced vacuum evaporation and the resulting residue may be purified using a silica gel column or HPLC.

### Pharmaceutical Compositions

This invention also provides a pharmaceutical composition including at least one of the compounds as described herein or a pharmaceutically-acceptable salt or solvate thereof, and a pharmaceutically-acceptable carrier.

In yet another aspect, the present invention provides a pharmaceutical composition including at least one compound selected from the group consisting of compounds of Formula I as described herein and a pharmaceutically-acceptable carrier or diluent.

In certain embodiments, the composition is in the form of a hydrate, solvate or pharmaceutically-acceptable salt. The composition can be administered to the subject by any suitable route of administration, including, without limitation, oral and parenteral.

The phrase “pharmaceutically-acceptable carrier” as used herein means a pharmaceutically-acceptable material, composition or vehicle, such as a liquid or solid filler, diluent, excipient, solvent, or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically-acceptable salts. The term “pharmaceutically-acceptable salt,” in this respect, refers to the relatively non-toxic, inorganic and organic acid salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. (See, for example, Berge et al., (1977) “Pharmaceutical Salts”, J. Pharm. Sci. 66:1-19.)

The pharmaceutically-acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like. See, e.g., Berge et al. (supra).

Wetting agents, emulsifiers, and lubricants, such as sodium lauryl sulfate, magnesium stearate, and polyethylene oxide-polybutylene oxide copolymer, as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also include buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions, to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically-acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the proper medium. Absorption enhancers can also be used to increase the flux of the pharmaceutical agents of the invention across the skin. The rate of such flux can be controlled, by either providing a rate-controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration include one or more compounds of the invention in combination with one or more pharmaceutically-acceptable sterile isotonic aqueous or nonaqueous solutions, dispersions, suspensions, or emulsions; or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers where the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot-injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically-acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism which can tolerate the compounds.

The invention also provides a pharmaceutical pack or kit including one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject

In yet another aspect, the present invention provides a method for treating a condition in a mammalian species in need thereof, the method including administering to the mammalian species a therapeutically effective amount of at least one compound selected from the group consisting of compounds of Formula I, or a pharmaceutically-acceptable salt thereof, where the condition is selected from the group consisting of cancer, an immunological disorder, a central nervous system (CNS) disorder, an inflammatory disorder, a gastroenterological disorder, a metabolic disorder, a cardiovascular disorder, and a kidney disease.

In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In some embodiments, the inflammatory disorder is an inflammatory skin condition, arthritis, psoriasis, spondylitis, parodontitits, or an inflammatory neuropathy. In some embodiments, the gastroenterological disorder is an inflammatory bowel disease such as Crohn's disease or ulcerative colitis.

In some embodiments, the immunological disorder is transplant rejection or an autoimmune disease (e.g., rheumatoid arthritis, multiple sclerosis, systemic lupus erythematosus, or type I diabetes mellitus). In some embodiments, the CNS disorder is Alzheimer's disease.

In some embodiments, the metabolic disorder is obesity or type II diabetes mellitus. In some embodiments, the cardiovascular disorder is an ischemic stroke. In some embodiments, the kidney disease is chronic kidney disease, nephritis, or chronic renal failure.

In some embodiments, the mammalian species is human.

In some embodiments, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, inflammatory bowel disease, obesity, type II diabetes mellitus, ischemic stroke, chronic kidney disease, nephritis, chronic renal failure, and a combination thereof.

In yet another aspect, a method of blocking Kv1.3 potassium channel in a mammalian species in need thereof is described, including administering to the mammalian species a therapeutically effective amount of at least one compound of Formula I, or a pharmaceutically-acceptable salt thereof.

In some embodiments, the compounds described herein is selective in blocking the Kv 1.3 potassium channels with minimal or no off-target inhibition activities against other potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically-acceptable solutions, which may routinely contain pharmaceutically-acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, age and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically-acceptable salt. When used in medicine the salts should be pharmaceutically acceptable, but non-pharmaceutically-acceptable salts can conveniently be used to prepare pharmaceutically-acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, Pa.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids, or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### Assays for Determining the Effectiveness of Kv1.3 Potassium Channel Blockers

In some embodiments, the compounds as described herein are tested for their activities against Kv1.3 potassium channel. In some embodiments, the compounds as described herein are tested for their Kv1.3 potassium channel electrophysiology. In some embodiments, the compounds as described herein are tested for their hERG electrophysiology.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Examples 1-5 describe various intermediates used in the syntheses of representative compounds of Formula I disclosed herein.

### Example 1. Intermediate 1 (2-bromo-3,4-dichloro-1-methoxybenzene) and Intermediate 2 (1-bromo-4,5-dichloro-2-methoxybenzene)

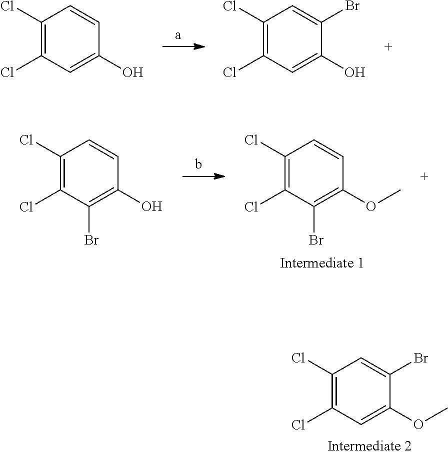

Step a:

To a stirred solution of 3,4-dichlorophenol (100.00 g, 613.49 mmol) in DCM (1000 mL) was added Br₂ (98.04 g, 613.49 mmol) dropwise at 0° C. under nitrogen atmosphere. The reaction solution was stirred for 16 h at room temperature under nitrogen atmosphere. The reaction was quenched with saturated aq. Na₂S₂O₃ (500 mL) at 0° C. The resulting mixture was extracted with EA (6×400 mL). The combined organic layers were washed with brine (2×400 mL) and dried over anhydrous Na₂SO₄. After filtration, the filtrate was concentrated under reduced pressure to afford a mixture of 2-bromo-4,5-dichlorophenol and 2-bromo-3,4-dichlorophenol (100 g, crude) as a yellow oil. The crude product was used in the next step directly without further purification.

Step b:

To a crude mixture of 2-bromo-4,5-dichlorophenol and 2-bromo-3,4-dichlorophenol (32 g, 125.04 mmol, 1 equiv.) and K₂CO₃ (54.9 g, 396.87 mmol, 3 equiv.) in ACN (210 mL) was added MeI (16.5 mL, 116.05 mmol, 2 equiv.) dropwise at 0° C. The reaction mixture was stirred at 50° C. for 4 h. The reaction mixture was filtered and concentrated. The residue was purified by silica gel column chromatography, eluted with PE to afford Intermediate 1 (2-bromo-3,4-dichloro-1-methoxybenzene) (8.7 g, 25.7%) as a white solid: ¹H NMR (300 MHz, CDCl3) δ 7.40 (dd, J=9.0, 1.1 Hz, 1H), 6.79 (d, J=8.9 Hz, 1H), 3.92 (s, 3H); and Intermediate 2 (1-bromo-4,5-dichloro-2-methoxybenzene) (24.3 g, 71.77%) as a white solid: ¹H NMR (300 MHz, CDCl₃) δ 7.64 (s, 1H), 6.99 (s, 1H), 3.91 (s, 3H).

### Example 2. Intermediate 3 ((2,3-dichloro-6-methoxyphenyl)boronic acid)

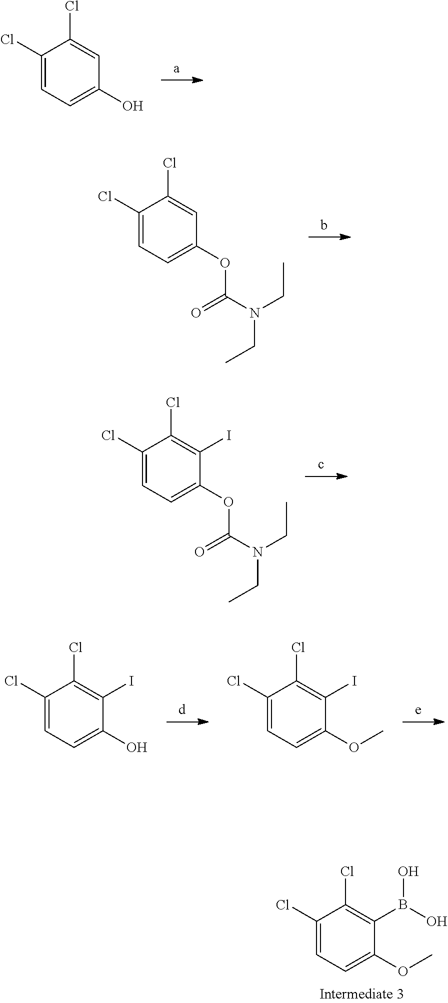 [SMILES: Oc1ccc(Cl)c(Cl)c1 ; CCN(CC)C(=O)Oc1ccc(Cl)c(Cl)c1 ; CCN(CC)C(=O)Oc1ccc(Cl)c(Cl)c1I ; Oc1ccc(Cl)c(Cl)c1I ; COc1ccc(Cl)c(Cl)c1I ; COc1ccc(Cl)c(Cl)c1B(O)O]

Step a:

To a stirred solution of 3,4-dichlorophenol (120 g, 0.74 mol) in THE (400 mL) was added NaOH (75 g, 1.88 mol) in portions at room temperature under nitrogen atmosphere, followed by stirring for 30 min. To this was added N,N-diethylcarbamoyl chloride (150 g, 1.11 mol) over 40 min, followed by stirring for 15 h. The reaction mixture was poured into water (1.5 L) and extracted with PE (2×800 mL). The combined organic phase was washed with brine (500 mL) and dried over Na₂SO₄. After filtration, the filtrate was concentrated under reduced pressure to afford 3,4-dichlorophenyl N,N-diethylcarbamate as a yellow oil (213 g, crude): LCMS (ESI) calculated for C₁₁H₁₃Cl₂NO₂ [M+H]⁺: 262, 264 (3:2), found 262, 264 (3:2); ¹H NMR (400 MHz, CDCl₃) δ 7.43 (d, J=8.8 Hz, 1H), 7.30 (d, J=2.7 Hz, 1H), 7.03 (dd, J=8.8, 2.7 Hz, 1H), 3.50-3.34 (m, 4H), 1.32-1.17 (m, 6H).

Step b:

To a solution of DIPA (32 g, 0.32 mol) in THE (400 mL) was added dropwise n-BuLi (131 mL, 0.33 mmol) at −65° C. under nitrogen atmosphere. The resulting mixture was stirred for 1 h. To this was added a solution of 3,4-dichlorophenyl N,N-diethylcarbamate (77 g, 0.29 mol) in THE (200 mL) dropwise, followed by stirring for 1 h. To this was added a solution of 12 (82 g, 0.32 mol) in THE (200 mL) dropwise over 1 h. The resulting mixture was stirred for additional 30 min at −65° C. The reaction was quenched by the addition of aq. NH₄Cl (300 mL) at room temperature. The resulting mixture was extracted with EA (3×400 mL). The combined organic layers were washed with brine (500 mL) and dried over anhydrous Na₂SO₄. After filtration, the filtrate was concentrated under reduced pressure. Three additional batches (3×77 g of 3,4-dichlorophenyl N,N-diethylcarbamate) were reacted, worked up and then combined with the previous one. The resulting residue was slurried in PE (500 mL), and then filtered to afford 300 g of 3,4-dichloro-2-iodophenyl N,N-diethylcarbamate. The filtrate was purified with silica gel column chromatography, eluted with PE/EA (50/1) to afford another 75 g of pure product. 3,4-dichloro-2-iodophenyl N,N-diethylcarbamate (375 g, 83% over 2 steps) was obtained as an off-white solid: LCMS (ESI) calculated for C₁₁H₁₂Cl₂INO₃ [M+H]⁺: 388, 390 (3:2), found 388, 390 (3:2); ¹H NMR (400 MHz, CDCl₃) δ 7.48 (d, J=8.8 Hz, 1H), 7.08 (d, J=8.8 Hz, 1H), 3.55 (q, J=7.2 Hz, 2H), 3.42 (q, J=7.1 Hz, 2H), 1.34 (t, J=7.1 Hz, 3H), 1.25 (t, J=7.1 Hz, 3H).

Step c:

To a stirred solution of 3,4-dichloro-2-iodophenyl N,N-diethylcarbamate (200 g, 0.52 mol) in EtOH (1.50 L) was added NaOH (165 g, 4.1 mol) at room temperature. The resulting mixture was stirred for 1 h at 80° C. under nitrogen atmosphere. The reaction mixture was concentrated under reduced pressure. The residue was diluted with ice water (1.5 L). The mixture was then acidified with aq. HCl (6 N) to pH=3. The resulting mixture was extracted with EA (3×1 L). The combined organic layers were washed with brine (800 mL) and dried over anhydrous Na₂SO₄. After filtration, the filtrate was concentrated under reduced pressure to afford 3,4-dichloro-2-iodophenol as a brown oil (202 g, crude): LCMS (ESI) calculated for C₆H₃Cl₂IO [M−H]⁻: 287, 289 (3:2), found 287, 289 (3:2).

Step d:

To a stirred solution of 3,4-dichloro-2-iodophenol (220 g, 0.76 mol) in DMF (700 mL) was added K₂CO₃ (210 g, 1.52 mol) and MeI (119 g, 0.84 mol). The resulting mixture was stirred for 5 h at room temperature. Another batch (100 g of 3,4-dichloro-2-iodophenol) was reacted and combined with the previous one. The resulting mixture was diluted with water (5 L) at room temperature. The resulting mixture was then extracted with EA (3×1 L). The combined organic layers were washed with brine (4×400 mL) and dried over anhydrous Na₂SO₄. After filtration, the filtrate was concentrated under reduced pressure. The residue was slurried in PE (300 mL), and then filtered to afford 128 g of desired product. The filtrate was purified by silica gel column chromatography, eluted with PE/EA (40/1) to afford an additional 64 g of desired product. 1,2-dichloro-3-iodo-4-methoxybenzene (192 g, 78% over 2 steps) was obtained as a light-yellow solid: ¹H NMR (400 MHz, CDCl₃) δ 7.44 (d, J=8.9 Hz, 1H), 6.70 (d, J=8.9 Hz, 1H), 3.91 (s, 3H).

Step e:

To a solution of 1,2-dichloro-3-iodo-4-methoxybenzene (100 g, 0.33 mol) in THF (1.2 L) was added i-PrMgCl (182 mL, 0.36 mol) dropwise at 0° C. under nitrogen atmosphere. The reaction mixture was then stirred at 0° C. for 1 h. B(OMe)₃ (86 g, 0.83 mol) was added dropwise at 0° C. Then, the reaction mixture was allowed to warm to room temperature over 1 h and stirred at room temperature for an additional 1 h. Then, aq. H₂SO₄ (5%, 500 mL) was added dropwise at 0° C. The reaction mixture was stirred at room temperature for 30 min. The mixture was extracted with EA (2×500 mL). The organic layers were combined, washed with brine (500 mL) and dried over anhydrous Na₂SO₄. After filtration, the filtrate was concentrated. The residue was stirred in DCM (200 mL), and then filtered to afford Intermediate 3 ((2,3-dichloro-6-methoxyphenyl)boronic acid) as an off-white solid (55 g, 76%): LCMS (ESI) calculated for C₁₅H₁₆Cl₂N₂O₄ [M−H]⁻: 219, 221 (3:2), found 219, 221 (3:2); ¹H NMR (400 MHz, CDCl₃) δ 7.48 (d, J=8.8 Hz, 1H), 6.82 (d, J=8.9 Hz, 1H), 5.65 (s, 2H), 3.89 (s, 3H).

### Example 3. Intermediate 4 (1,2-di-tert-butyl (2S)-6-oxo-4-(trifluoromethanesulfonyloxy)-2,3-dihydropyridine-1,2-dicarboxylate)

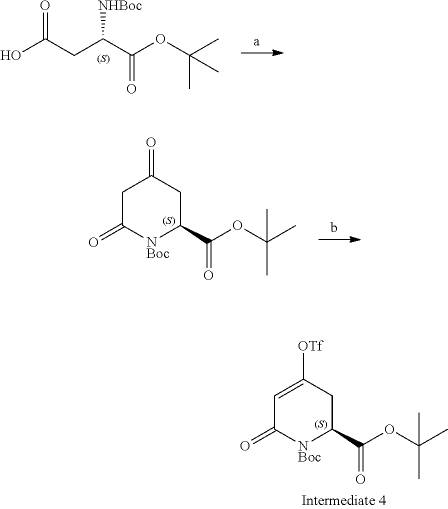

Step a:

EDCI (4.97 g, 25.92 mmol), DMAP (3.17 g, 25.92 mmol), and Meldrum's acid (2.49 g, 17.28 mmol) were added to a solution of (3S)-4-(tert-butoxy)-3-[(tert-butoxycarbonyl)amino]-4-oxobutanoic acid (5.0 g, 17.28 mmol) in DCM (70 mL) at 0° C. The mixture was stirred for 3 h at room temperature and then washed with aq. KHSO₄ (30 mL). The organic layer was dried over anhydrous Na₂SO₄, filtered, and concentrated under reduced pressure. The residue was dissolved in EA (170 mL) and refluxed overnight. The resulting mixture was cooled, washed with aq. KHSO₄ (60 mL) and brine (50 mL), dried over anhydrous Na₂SO₄, filtered and the filtrate concentrated under reduced pressure. The crude product was washed with DCM/PE (½, 25 mL) to afford 1,2-di-tert-butyl (2S)-4,6-dioxopiperidine-1,2-dicarboxylate as an off-white solid (3 g, 55%): LCMS (ESI) calculated for C₁₅H₂₃NO₆ [M+H−100]⁺: 214, found 214; ¹H NMR (400 MHz, CDCl₃) δ 5.09 (dd, J=6.9, 2.2 Hz, 1H), 3.55 (d, J=19.5 Hz, 1H), 3.39 (d, J=19.4 Hz, 1H), 3.04 (dd, J=17.6, 2.2 Hz, 1H), 2.85 (dd, J=17.6, 6.9 Hz, 1H), 1.57 (s, 9H), 1.48 (s, 9H).

Step b:

To a solution of 1,2-di-tert-butyl (2S)-4,6-dioxopiperidine-1,2-dicarboxylate (1.0 g, 3.19 mmol) in DCM (10 mL) was added DIPEA (1.67 mL, 12.90 mmol) dropwise at 0° C. under nitrogen atmosphere. To this was added triflic anhydride (1.08 g, 3.83 mmol) dropwise at 0° C., followed by stirring for 1 h at room temperature. The reaction mixture was quenched with 10 mL of aq. NaHCO₃. The aqueous phase was extracted with DCM (10 mL). The organic phases were combined, washed with brine (10 mL), and dried over anhydrous Na₂SO₄. After filtration, the filtrate was concentrated under reduced pressure to afford Intermediate 4 (1,2-di-tert-butyl (2S)-6-oxo-4-(trifluoromethanesulfonyloxy)-2,3-dihydropyridine-1,2-dicarboxylate) as a brown solid (2.6 g, crude): LCMS (ESI) calculated for C₁₄H₂₂F₃NO₈S [M+H]⁺: 446, found 446.

### Example 4. Intermediate 5 (methyl 4-(2,3-dichloro-6-methoxyphenyl)piperidine-2-carboxylate)

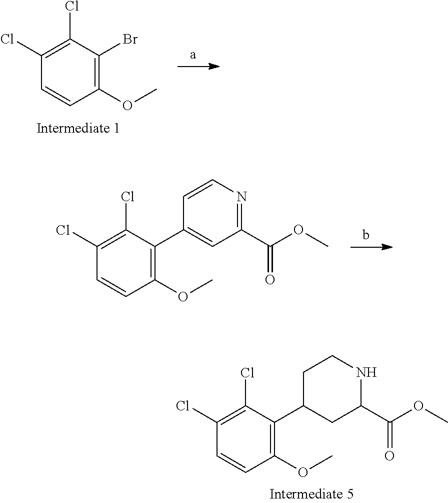 [SMILES: COc1ccc(Cl)c(Cl)c1Br ; COC(=O)c1cc(-c2c(OC)ccc(Cl)c2Cl)ccn1 ; COC(=O)C1CC(c2c(OC)ccc(Cl)c2Cl)CCN1]

Step a:

To a solution of 2-bromo-3,4-dichloro-1-methoxybenzene (Intermediate 1, Example 1) (5 g, 0.02 mmol, 1 equiv.) and methyl 4-(4,4,5,5-tetramethyl-1,3,2-dioxaborolan-2-yl)pyridine-2-carboxylate (6.2 g, 0.02 mmol, 1.2 equiv.) in dioxane and water were added Na₂CO₃ (6.2 g, 0.06 mmol, 3 equiv.) and Pd(dppf)Cl₂.CH₂Cl₂ (3.2 g, 0.2 equiv.). After stirring for 3 h at 80° C. under a nitrogen atmosphere, the resulting mixture was concentrated under reduced pressure. The residue was purified by silica gel column chromatography, eluted with PE/EA (3:1) to afford methyl 4-(2,3-dichloro-6-methoxyphenyl)pyridine-2-carboxylate (1 g, 16.4%) as a light-yellow solid: LCMS (ESI) calculated for C₁₄H₁₁Cl₂NO₃ [M+H]⁺: 312, 314 (3:2), found 312,314 (3:2). ¹H NMR (400 MHz, CD₃OD) δ 8.78 (d, J=5.0 Hz, 1H), 8.08 (s, 1H), 7.66-7.57 (m, 2H), 7.16 (d, J=9.0 Hz, 1H), 4.01 (s, 3H), 3.78 (s, 3H).

Step b:

To a solution of PtO₂ (65.5 mg, 0.29 mmol, 0.3 equiv.) and methyl 4-(2,3-dichloro-6-methoxyphenyl)pyridine-2-carboxylate (300 mg, 0.96 mmol, 1 equiv.) in MeOH was added HCl (6 M, 1 mL) in portions at room temperature. The resulting mixture was stirred for 4 days at 30° C. under hydrogen atmosphere. The solid was filtered out and washed with MeOH (3×10 mL). The filtrate was concentrated under reduced pressure to afford Intermediate 5 (methyl 4-(2,3-dichloro-6-methoxyphenyl)piperidine-2-carboxylate) (200 mg, 52.32%) as a yellow oil: LCMS (ESI) calculated for C₁₄H₁₇Cl₂NO₃ [M+H]⁺: 318, 320 (3:2), found 318, 320 (3:2). ¹H NMR (400 MHz, CD₃OD) δ 7.38 (d, J=8.9 Hz, 1H), 6.96 (d, J=9.0 Hz, 1H), 3.86 (s, 3H), 3.74 (s, 3H), 3.67-3.58 (m, 1H), 3.47 (dd, J=11.9, 3.0 Hz, 1H), 3.26-3.16 (m, 1H), 2.76 (td, J=12.4, 2.9 Hz, 1H), 2.45-2.27 (m, 2H), 1.90 (d, J=12.7 Hz, 1H), 1.51 (d, J=13.1 Hz, 1H).

### Example 5. Intermediate 6 (methyl (2S,4R)-4-(2,3-dichloro-6-methoxyphenyl)piperidine-2-carboxylate)

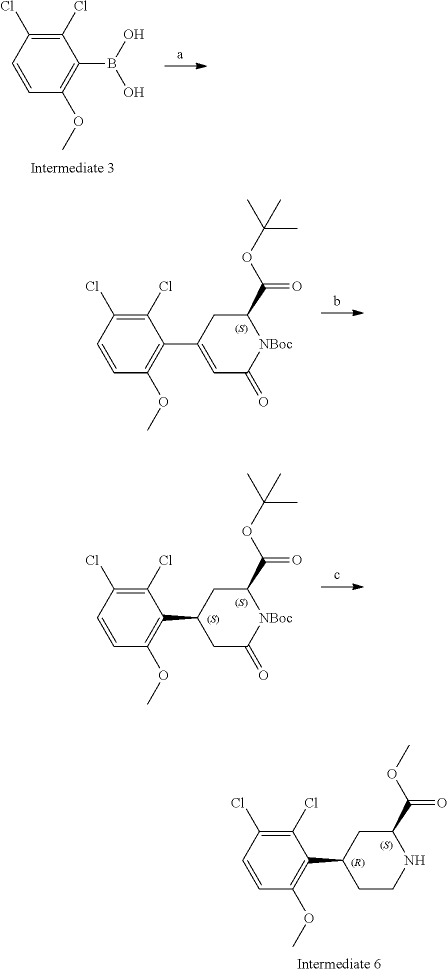

Step a:

To a mixture of 1,2-di-tert-butyl (2S)-6-oxo-4-(trifluoromethanesulfonyloxy)-2,3-dihydropyridine-1,2-dicarboxylate (Intermediate 4, Example 3) (2.52 g, crude), 2,3-dichloro-6-methoxyphenyl)boronic acid (Intermediate 3, Example 2) (700 mg, 3.17 mmol), and Na₂CO₃ (1.01 g, 9.51 mmol) in dioxane (20 mL) and H₂O (5 mL) was added Pd(dppf)Cl₂.CH₂Cl₂ (130 mg, 0.16 mmol) in one portion at room temperature. The suspension was degassed under vacuum and purged with nitrogen 3 times. The reaction was stirred at 80° C. for 3 h under nitrogen atmosphere and then concentrated under reduced pressure. The residue was dissolved in EA (30 mL) and washed with brine (2×15 mL). The organic phase was dried over anhydrous Na₂SO₄, filtered, and concentrated under reduced pressure. The residue was purified with silica gel column chromatography, eluted with PE/EA (5/1) to afford 1,2-di-tert-butyl (2S)-4-(2,3-dichloro-6-methoxyphenyl)-6-oxo-2,3-dihydropyridine-1,2-dicarboxylate as a light-yellow foam (900 mg, 60%): LCMS (ESI) calculated for C₂₂H₂₁Cl₂N₇O₆ [M+H−100]⁺: 372, 374 (3:2), found 372, 374 (3:2); ¹H NMR (400 MHz, Chloroform-d) δ 7.41 (d, J=8.9 Hz, 1H), 6.79 (d, J=9.0 Hz, 1H), 5.92 (d, J=2.7 Hz, 1H), 4.94 (dd, J=7.2, 1.8 Hz, 1H), 3.78 (s, 3H), 3.13 (d, J=18.5 Hz, 1H), 2.89 (d, J=18.3 Hz, 1H), 1.59 (s, 9H), 1.49 (s, 9H).

Step b:

To a solution of 1,2-di-tert-butyl (2S)-4-(2,3-dichloro-6-methoxyphenyl)-6-oxo-2,3-dihydropyridine-1,2-dicarboxylate (900 mg, 1.91 mmol) in EA (50 mL) and AcOH (0.50 mL) was added PtO₂ (150 mg, 0.66 mmol) under nitrogen atmosphere. The suspension was degassed under vacuum and purged 3 times with hydrogen. The mixture was stirred under hydrogen (1.5 atm) at room temperature for 16 h. Then, the reaction was filtered and concentrated under reduced pressure. The residue was purified by silica gel column chromatography, eluted with PE/EA (5/1) to afford 1,2-di-tert-butyl (2S,4S)-4-(2,3-dichloro-6-methoxyphenyl)-6-oxopiperidine-1,2-dicarboxylate as a colorless foam (550 mg, 61%): LCMS (ESI) calculated for C₂₂H₂₉Cl₂NO₆ [M+H−100]⁺: 474, 476 (3:2), found 474, 476 (3:2); ¹H NMR (400 MHz, CDCl₃) δ 7.35 (d, J=9.0 Hz, 1H), 6.77 (d, J=9.0 Hz, 1H), 4.61 (dd, J=8.7, 7.2 Hz, 1H), 4.01-3.87 (m, 1H), 3.82 (s, 3H), 3.40-3.23 (m, 1H), 2.66-2.48 (m, 2H), 2.36-2.27 (m, 1H), 1.56 (s, 9H), 1.49 (s, 9H).

Step c:

To a solution of 1,2-di-tert-butyl (2S,4S)-4-(2,3-dichloro-6-methoxyphenyl)-6-oxopiperidine-1,2-dicarboxylate (550 mg, 1.16 mmol) in THE (10 mL) was added BH₃.Me₂S (0.21 mL, 2.11 mmol) under nitrogen atmosphere at 0° C. Then, the reaction was stirred under nitrogen atmosphere at room temperature for 4 h. Then, 10 mL of MeOH at 0° C. was added dropwise and the resulting mixture stirred for 1 h. To this was added 6 mL of aq. HCl (6 M). The reaction was stirred at room temperature for 12 h. The reaction was concentrated under reduced pressure. The residue was purified with reverse phase HPLC to afford Intermediate 6 (methyl (2S,4R)-4-(2,3-dichloro-6-methoxyphenyl)piperidine-2-carboxylate) (125 mg, 34%) as colorless oil: LCMS (ESI) calculated for C₁₄H₁₇Cl₂NO₃ [M+H]⁺: 318, 320 (3:2), found 318, 320 (3:2); ¹H NMR (400 MHz, CD₃OD) δ 7.38 (d, J=8.9 Hz, 1H), 6.96 (d, J=9.0 Hz, 1H), 3.86 (s, 3H), 3.74 (s, 3H), 3.69-3.53 (m, 1H), 3.47 (dd, J=11.9, 3.0 Hz, 1H), 3.25-3.16 (m, 1H), 2.76 (td, J=12.4, 2.9 Hz, 1H), 2.45-2.25 (m, 2H), 1.90 (d, J=12.7 Hz, 1H), 1.51 (d, J=13.0 Hz, 1H).

Examples 6-8 describe the syntheses and/or characterization data of representative compounds of Formula I disclosed herein.

### Example 6. Compound 1 ((8R,9aS)-2-(azetidin-3-yl)-8-(2,3-dichloro-6-hydroxyphenyl)-octahydro-1H-pyrido[1,2-a]pyrazine-1,4-dione)

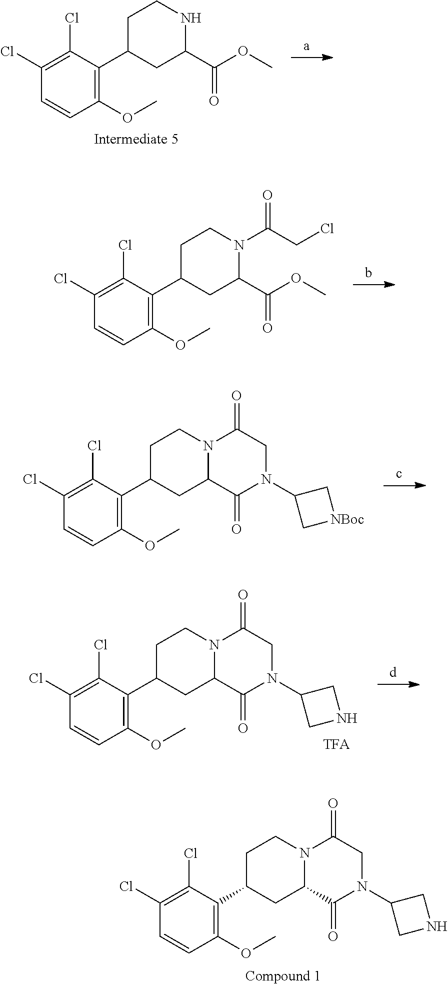 [SMILES: COC(=O)C1CC(c2c(OC)ccc(Cl)c2Cl)CCN1 ; COC(=O)C1CC(c2c(OC)ccc(Cl)c2Cl)CCN1C(=O)CCl ; COc1ccc(Cl)c(Cl)c1C1CCN2C(=O)CN(C3CN(C(=O)OC(C)(C)C)C3)C(=O)C2C1 ; COc1ccc(Cl)c(Cl)c1C1CCN2C(=O)CN(C3CNC3)C(=O)C2C1 ; COc1ccc(Cl)c(Cl)c1[C@@H]1CCN2C(=O)CN(C3CNC3)C(=O)[C@@H]2C1]

Step a:

To a stirred solution of methyl 4-(2,3-dichloro-6-methoxyphenyl)piperidine-2-carboxylate (Intermediate 5, Example 4) (400 mg, 1.01 mmol, 80%) and TEA (509 mg, 5.03 mmol) in DCM (8 mL) was added 2-chloroacetyl chloride (170 mg, 1.51 mmol) at 0° C. under nitrogen atmosphere. The reaction mixture was stirred for 1 h at room temperature, then concentrated to afford methyl 1-(2-chloroacetyl)-4-(2,3-dichloro-6-methoxyphenyl)piperidine-2-carboxylate as light brown solid (500 mg, crude): LCMS (ESI) calculated for C16H18Cl3NO4 [M+H]+: 394, 396 (1:1), found 394, 396 (1:1).

Step b:

To a stirred solution of methyl 1-(2-chloroacetyl)-4-(2,3-dichloro-6-methoxyphenyl)piperidine-2-carboxylate (500 mg, 1.27 mmol) and TEA (385 mg, 3.80 mmol) in EtOH (10 mL) was added tert-butyl 3-aminoazetidine-1-carboxylate (327 mg, 1.90 mmol) at room temperature. The resulting reaction mixture was stirred for 16 h at 80° C., then concentrated under vacuum. The residue was dissolved in EA (20 mL). The solution was washed with brine (2×10 mL). The organic phase was dried over Na₂SO₄, filtered, and the filtrate was concentrated. The residue was purified with reverse phase HPLC to obtain tert-butyl 3-[8-(2,3-dichloro-6-methoxyphenyl)-1,4-dioxo-octahydro-1H-pyrido[1,2-a]pyrazin-2-yl]azetidine-1-carboxylate as light brown oil (285 mg, 45%): LCMS (ESI) calculated for C₂₃H₂₉Cl₂N₃O₅ [M+H]⁺: 498, 500 (3:2), found 498, 500 (3:2).

Step c:

To a stirred solution of tert-butyl 3-[8-(2,3-dichloro-6-methoxyphenyl)-1,4-dioxo-octahydro-1H-pyrido[1,2-a]pyrazin-2-yl]azetidine-1-carboxylate (285 mg, 0.29 mmol, 80%) in DCM (5 mL) was added BBr₃ (0.51 mL, 2.03 mmol) at room temperature. The reaction was stirred at room temperature for 3 h. The reaction mixture was quenched with water (10 mL). The pH value was adjusted to 7 by adding saturated aq. NaHCO₃ and the resulting solution was concentrated under vacuum. The residue was purified by Prep-HPLC with the following condition: Column: Xselect CSH OBD Column 30×150 mm, 5 μm; Mobile Phase A: Water (0.1% FA), Mobile Phase B: ACN; Flow rate: 60 mL/min; Gradient: 7% B to 25% B in 9 min; Detector: UV 220 nm; Retention time: 8.20 min. The fractions containing desired product were combined and concentrated under reduced pressure to afford 2-(azetidin-3-yl)-8-(2,3-dichloro-6-hydroxyphenyl)-octahydro-1H-pyrido[1,2-a]pyrazine-1,4-dione trifluoroacetic acid as an off-white solid (120 mg, 86%): LCMS (ESI) calculated for C₁₇H₁₉Cl₂N₃O₃ [M+H]⁺: 384, 386 (3:2), found 384, 386 (3:2). ¹H NMR (400 MHz, Methanol-d₄) δ 7.21 (d, J=8.8 Hz, 1H), 6.72 (d, J=8.8 Hz, 1H), 5.04-4.95 (m, 1H), 4.75-4.60 (m, 1H), 4.30-4.04 (m, 3H), 3.98-3.83 (m, 2H), 3.82-3.58 (m, 3H), 2.87-2.71 (m, 1H), 2.61-2.40 (m, 2H), 2.14 (d, J=12.9 Hz, 1H), 1.64 (d, J=13.2 Hz, 1H).

Step d:

2-(azetidin-3-yl)-8-(2,3-dichloro-6-hydroxyphenyl)-octahydro-1H-pyrido[1,2-a]pyrazine-1,4-dione (120 mg, 0.31 mmol) was separated with chiral-HPLC with the following conditions: Column: CHIRALPAK IG, 2×25 cm, 5 um; Mobile Phase A: Hex (0.2% IPA), Mobile Phase B: EtOH; Flow rate: 20 mL/min; Gradient: 30% B to 30% B in 21 min; Detector: UV 220/254 nm; Retention time: 17.719 min. The fractions containing the desired product were combined and concentrated under reduced pressure to afford Compound 1 ((8R,9aS)-2-(azetidin-3-yl)-8-(2,3-dichloro-6-hydroxyphenyl)-octahydro-1H-pyrido[1,2-a]pyrazine-1,4-dione) as an off-white solid (30 mg, 25%): LCMS (ESI) calculated for C₁₇H₁₉Cl₂N₃O₃ [M+H]⁺: 384, 386 (3:2), found 384, 386 (3:2). ¹H NMR (400 MHz, Methanol-d₄) δ¹H NMR (400 MHz, Methanol-d₄) δ 7.21 (d, J=8.8 Hz, 1H), 6.73 (d, J=8.8 Hz, 1H), 5.02-4.93 (m, 1H), 4.75-4.63 (m, 1H), 4.29-4.04 (m, 3H), 4.04-3.90 (m, 2H), 3.87-3.67 (m, 3H), 2.86-2.73 (m, 1H), 2.60-2.40 (m, 2H), 2.15 (d, J=12.9 Hz, 1H), 1.63 (d, J=13.2 Hz, 1H).

### Example 7. Compound 2 ((3R,8R,9aS)-8-(2,3-dichloro-6-hydroxyphenyl)-3-(hydroxymethyl)-hexahydro-2H-pyrido[1,2-a]pyrazine-1,4-dione)

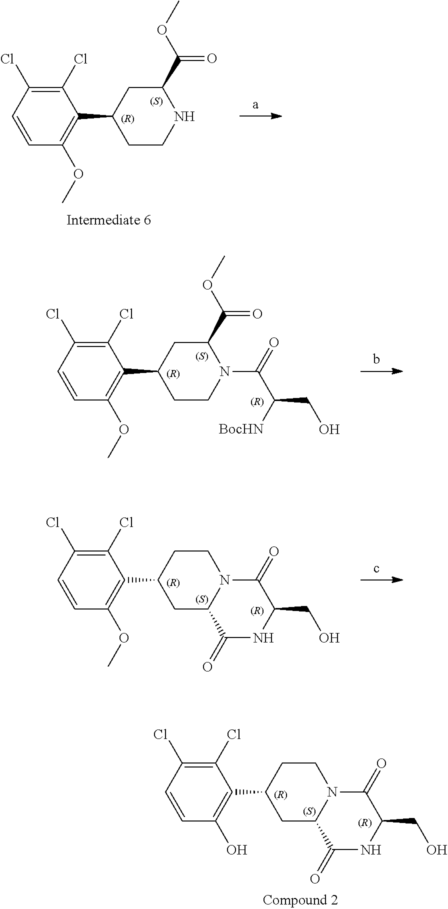

Step a:

To a stirred solution of (2R)-2-[(tert-butoxycarbonyl)amino]-3-hydroxypropanoic acid (97 mg, 0.47 mmol) in DMF (2 mL) were added EDCI (113 mg, 0.59 mmol) and HOBT (80 mg, 0.59 mmol) at room temperature. TEA (119 mg, 1.18 mmol) and methyl (2S,4R)-4-(2,3-dichloro-6-methoxyphenyl)piperidine-2-carboxylate (Intermediate 6, Example 5) (125 mg, 0.40 mmol) were then added and the resulting mixture was stirred for 12 h, then poured into H₂O (10 mL) and extracted with EA (3×5 mL). The combined organic phase was washed with brine (3×5 mL), dried over anhydrous Na₂SO₄, and filtered and concentrated under reduced pressure. The residue was purified with Prep-HPLC with the follow conditions: Column: Xselect CSH OBD Column 30×150 mm, 5 μm; Mobile Phase A: Water (0.05% TFA), Mobile Phase B: ACN; Flow rate: 60 mL/min; Gradient: 35% B to 60% B in 8 min; Detector: UV 220 nm; Retention time: 7.12. The fractions containing the desired product were collected and concentrated under reduced pressure to afford methyl (2S,4R)-1-[(2R)-2-[(tert-butoxycarbonyl)amino]-3-hydroxypropanoyl]-4-(2,3-dichloro-6-methoxyphenyl)piperidine-2-carboxylate as a light-yellow foam (35 mg, 18%): LCMS (ESI) calculated for C₂₂H₃₀Cl₂N₂O₇ [M+H]⁺: 505, 507 (3:2), found 505, 507 (3:2).

Step b:

To a stirred solution of methyl (2S,4R)-1-[(2R)-2-[(tert-butoxycarbonyl)amino]-3-hydroxypropanoyl]-4-(2,3-dichloro-6-methoxyphenyl)piperidine-2-carboxylate (35 mg, 0.07 mmol) in DCM (1 mL) was added TFA (0.5 mL, 6.73 mmol) at room temperature. The resulting mixture was stirred for 30 min and concentrated under reduced pressure. The residue was dissolved in EtOH (3 mL). TEA (21 mg, 0.21 mmol) was then added and the reaction mixture was stirred at 80° C. for 12 h. The reaction mixture was concentrated to afford (3R,8R,9aS)-8-(2,3-dichloro-6-methoxyphenyl)-3-(hydroxymethyl)-hexahydro-2H-pyrido[1,2-a]pyrazine-1,4-dione as light-yellow oil (80 mg, crude): LCMS (ESI) calculated for C₁₆H₁₈Cl₂N₂O₄ [M+H]⁺: 373, 375 (3:2), found 373, 375 (3:2).

Step c:

To a stirred solution of (3R,8R,9aS)-8-(2,3-dichloro-6-methoxyphenyl)-3-(hydroxymethyl)-hexahydro-2H-pyrido[1,2-a]pyrazine-1,4-dione (80 mg, crude) in DCM (2 mL) was added BBr₃ (0.2 mL) at room temperature. The reaction mixture was stirred for 2 h, then added dropwise to 3 mL of MeOH at 0° C. The resulting mixture was concentrated under reduced pressure. The residue was purified with Prep-HPLC with the follow conditions: Column: XBridge Prep C18 OBD Prep Column, 19×150 mm, 5 μm; Mobile Phase A: water (plus 0.05% TFA), Mobile Phase B: ACN; Flow rate: 60 mL/min; Gradient: 15% B to 40% B in 7 min; Detector: UV 220 nm; Retention time: 6.58 min. The fractions containing the desired product were collected and concentrated under reduced pressure to afford Compound 2 ((3R,8R,9aS)-8-(2,3-dichloro-6-hydroxyphenyl)-3-(hydroxymethyl)-hexahydro-2H-pyrido[1,2-a]pyrazine-1,4-dione) as an off-white solid (12.3 mg): LCMS (ESI) calculated for C₁₅H₁₆Cl₂N₂O₄ [M+H]⁺: 359, 361 (3:2), found 359, 361 (3:2); ¹H NMR (400 MHz, CD₃OD) δ 7.22 (d, J=8.8 Hz, 1H), 6.73 (d, J=8.8 Hz, 1H), 4.79-4.68 (m, 1H), 4.14-3.96 (m, 3H), 3.75 (dd, J=11.0, 2.6 Hz, 2H), 2.78 (td, J=13.2, 3.0 Hz, 1H), 2.59-2.41 (m, 2H), 2.26-2.15 (m, 1H), 1.66 (d, J=13.3 Hz, 1H).

### Example 8. The Following Compounds were Made in Analogous Fashion to that of Compound 1 (Example 6) or Compound 2 (Example 7), and/or by a Method Known in the Art

TABLE 1a

| Compound | | | |
|---|---|---|---|
| Number | Structure | Chemical Name | MS (M + H)⁺ & ¹H NMR |
| 3 | 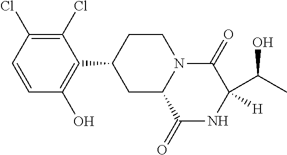 [SMILES: C[C@H](O)[C@H]1NC(=O)[C@@H]2C[C@H](c3c(O)ccc(Cl)c3Cl)CCN2C1=O] | (3R,8R,9aS)-8-(2,3- dichloro-6- hydroxyphenyl)-3- [(1S)-1- hydroxyethyl]- hexahydro-2H- pyrido[1,2- a]pyrazine-1,4-dione | [M + H]⁺_{:} 373, 375 (3:2); ¹H NMR (400 MHz, CD₃OD) δ 7.22 (d, J = 8.8 Hz, 1H), 6.72 (d, J = 8.8 Hz, 1H), 4.78-4.68 (m, 1H), 4.32-4.23 (m, 1H), 4.06 (dd, J = 12.2, 3.4 Hz, 1H), 3.86-3.80 (m, 1H), 3.78-3.66 (m, 1H), 2.77 (td, J = 13.3, 3.1 Hz, 1H), 2.56-2.41 (m, 2H), 2.27-2.19 (m, 1H), 1.66 (d, J = 13.2 Hz, 1H), 1.27 (d, J = 6.6 Hz, 3H). |
| 4 | 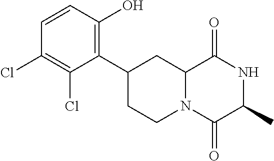 [SMILES: C[C@@H]1NC(=O)C2CC(c3c(O)ccc(Cl)c3Cl)CCN2C1=O] | (3S)-8-(2,3-dichloro- 6-hydroxyphenyl)-3- methyl-octahydro- 1H-pyrido[1,2- a]pyrazine-1,4-dione | [M + H]⁺: 343, 345 (3:2); ¹H NMR (400 MHz, CD₃OD) δ 7.22 (dd, J = 8.8, 2.0 Hz, 1H), 6.72 (d, J = 8.8 Hz, 1H), 4.74-4.63 (m, 1H), 4.19-4.04 (m, 2H), 3.87-3.73 (m, 1H), 2.79 (td, J = 13.1, 3.0 Hz, 1H), 2.64-2.40 (m, 2H), 2.19-2.12 (m, 1H), 1.681.59 (m, 1H), 1.50 (dd, J = 11.1, 6.9 Hz, 3H). |
| 5 | 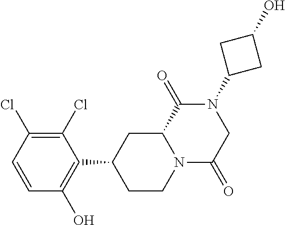 | 8-(2,3-dichloro-6- hydroxyphenyl)-2-[3- hydroxycyclobutyl]- hexahydropyrido[1,2- a]pyrazine-1,4-dione isomer 4 | [M + H]⁺: 399, 401 (3:2); ¹H NMR (400 MHz, CD₃OD) δ 7.21 (d, J = 8.8 Hz, 1H), 6.72 (d, J = 8.8 Hz, 1H), 5.16-5.00 (m, 1H), 4.75-4.64 (m, 1H), 4.42-4.32 (m, 1H), 4.21-4.05 (m, 3H), 3.85-3.70 (m, 1H), 2.79 (td, J = 13.2, 3.1 Hz, 1H), 2.66- 2.38 (m, 4H), 2.29-2.12 (m, 3H), 1.74- 1.56 (m, 1H). |
| 6 | 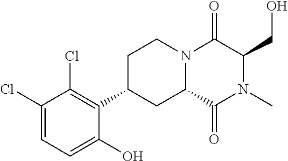 [SMILES: CN1C(=O)[C@@H]2C[C@H](c3c(O)ccc(Cl)c3Cl)CCN2C(=O)[C@H]1CO] | (3R,8R,9aS)-8-(2,3- dichloro-6- hydroxyphenyl)-3- (hydroxymethyl)-2- methyl- hexahydropyrido[1,2- a]pyrazine-1,4-dione | [M + H]⁺: 373, 375 (3:2); ¹H NMR (400 MHz, CD₃OD) δ 7.21 (d, J = 8.8 Hz, 1H), 6.72 (d, J = 8.8 Hz, 1H), 4.78-4.70 (m, 1H), 4.08 (dd, J = 12.2, 3.4 Hz, 1H), 4.05- 4.02 (m, 1H), 4.00-3.96 (m, 2H), 3.80- 3.63 (m, 1H), 3.00 (s, 3H), 2.78 (td, J = 13.3, 3.2 Hz, 1H), 2.54-2.39 (m, 2H), 2.32-2.24 (m, 1H), 1.70-1.61 (m, 1H). |
| 7 | 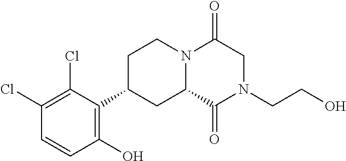 [SMILES: O=C1[C@@H]2C[C@H](c3c(O)ccc(Cl)c3Cl)CCN2C(=O)CN1CCO] | (8R,9aS)-rel-8-(2,3- dichloro-6- hydroxyphenyl)-2-(2- hydroxyethyl)hexahydro- 4H-pyrido[1,2- a]pyrazine-1,4(6H)- dione | [M + H]⁺: 373, 375 (3:2); ¹H NMR (400 MHz, CD₃OD) δ 7.22 (d, J = 8.8 Hz, 1H), 6.73 (d, J = 8.8 Hz, 1H), 4.74-4.65 (m, 1H), 4.33-4.09 (m, 3H), 3.84-3.71 (m, 3H), 3.59-3.47 (m, 2H), 2.80 (td, J = 13.1, 3.0 Hz, 1H), 2.62-2.40 (m, 2H), 2.21-2.13 (m, 1H), 1.64 (d, J = 13.4 Hz, 1H). |
| 8 | 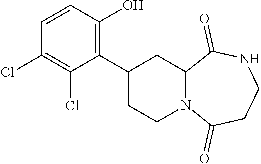 [SMILES: O=C1NCCC(=O)N2CCC(c3c(O)ccc(Cl)c3Cl)CC12] | 9-(2,3-dichloro-6- hydroxyphenyl)- decahydropyrido[1,2- a][1,4]diazepine-1,5- dione | [M + H]⁺_{:} 343, 345 (3:2); ¹1H NMR (400 MHz, CD₃OD) δ 7.22 (d, J = 8.7 Hz, 1H), 6.75 (d, J = 8.7 Hz, 1H), 4.59 (dd, J = 12.3, 4.0 Hz, 1H), 4.07-3.94 (m, 1H), 3.91-3.76 (m, 1H), 3.73-3.61 (m, 1H), 3.61-3.48 (m, 1H), 3.46-3.36 (m, 1H), 3.09-2.93 (m, 2H), 2.80-2.68 (m, 1H), 2.33-2.20 (m, 1H), 1.91-1.77 (m, 2H). |
| 9 | 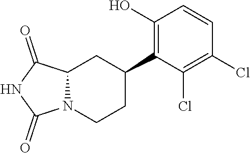 [SMILES: O=C1NC(=O)N2CC[C@H](c3c(O)ccc(Cl)c3Cl)C[C@@H]12] | 7-(2,3-dichloro-6- hydroxyphenyl)tetrahydro- imidazo[1,5- a]pyridine- 1,3(2H,5H)-dione diastereoisomer 1 | [M + H]⁺: 315, 317 (3:2); ¹H NMR (400 MHz, CD₃OD) δ 7.22 (d, J = 8.8 Hz, 1H), 6.73 (d, J = 8.8 Hz, 1H), 4.16 (dd, J = 13.3, 4.9 Hz, 1H), 4.10 (dd, J = 12.1, 4.4 Hz, 1H), 3.76-3.61 (m, 1H), 3.03 (td, J= 13.1, 3.6 Hz, 1H), 2.50-2.34 (m, 2H), 2.04-1.95 (m, 1H), 1.67-1.57 (m, 1H). |
| 10 | 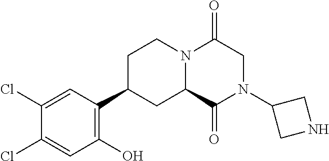 [SMILES: O=C1[C@H]2C[C@@H](c3cc(Cl)c(Cl)cc3O)CCN2C(=O)CN1C1CNC1] | (8S,9aR)-2-(azetidin- 3-yl)-8-(4,5-dichloro- 2- hydroxyphenyl)hexahydro- 4H-pyrido[1,2- a]pyrazine-1,4(6H)- dione | [M + H]⁺: 384, 386 (3:2); ¹H NMR (400 MHz, CD₃OD) δ 7.21 (s, 1H), 6.93 (s, 1H), 5.03-4.91 (m, 1H), 4.77-4.64 (m, 1H), 4.26-4.07 (m, 3H), 3.98-3.86 (m, 2H), 3.77-3.66 (m, 2H), 3.31-3.22 (m, 1H), 2.81 (td, J = 13.1, 2.9 Hz, 1H), 2.45- 2.34 (m, 1H), 1.98-1.86 (m, 1H), 1.84- 1.56 (m, 2H). |
| 11 | 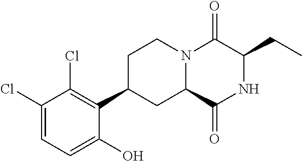 [SMILES: CC[C@H]1NC(=O)[C@H]2C[C@@H](c3c(O)ccc(Cl)c3Cl)CCN2C1=O] | (8S,9aR)-8-(2,3- dichloro-6- hydroxyphenyl)-3- (3R)-ethyl- hexahydro-2H- pyrido[1,2- a]pyrazine-1,4-dione | [M + H]⁺ 357, 359 (3:2); ¹H NMR (400 MHz, CD₃OD) δ 7.22 (d, J = 8.8 Hz, 1H), 6.72 (d, J = 8.8 Hz, 1H), 4.78-4.69 (m, 1H), 4.18-4.09 (m, 2H), 3.87-3.72 (m, 1H), 2.80 (td, J = 13.1, 3.0 Hz, 1H), 2.59- 2.40 (m, 2H), 2.23-2.13 (m, 1H), 2.11- 1.97 (m, 1H), 1.89-1.75 (m, 1H), 1.64 (d, J = 13.4 Hz, 1H), 0.93 (t, J = 7.4 Hz, 3H). |
| 12 | 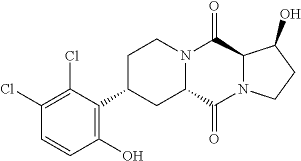 [SMILES: O=C1[C@H]2[C@@H](O)CCN2C(=O)[C@@H]2C[C@H](c3c(O)ccc(Cl)c3Cl)CCN12] | (1S,5aS,7R,11aR)-7- (2,3-dichloro-6- hydroxyphenyl)-1- hydroxyhexahydro- 1H-pyrido[1,2- a]pyrrolo[1,2- d]pyrazine- 5,11(5aH,11aH)- dione | [M + H]⁺: 385, 387 (3:2); ¹H NMR (400 MHz, DMSO-d₆) δ 10.14 (s, 1H), 7.31 (d, J = 8.8 Hz, 1H), 6.81 (d, J = 8.9 Hz, 1H), 5.24 (d, J = 4.4 Hz, 1H), 4.52-4.45 (m, 1H), 4.45-4.39 (m, 1H), 4.16-4.11 (m, 1H), 4.10-4.03 (m, 1H), 3.77-3.63 (m, 2H), 3.32-3.26 (m, 1H), 2.81-2.70 (m, 1H), 2.43-2.34 (m, 1H), 2.29-2.17 (m, 1H), 2.03-1.83 (m, 2H), 1.83-1.74 (m, 1H), 1.55-1.43 (m, 1H). |
| 13 | 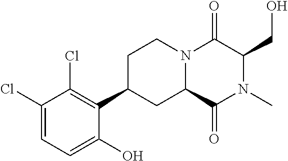 [SMILES: CN1C(=O)[C@H]2C[C@@H](c3c(O)ccc(Cl)c3Cl)CCN2C(=O)[C@H]1CO] | (3R,8S,9aR)-8-(2,3- dichloro-6- hydroxyphenyl)-3- (hydroxymethyl)-2- methyl- hexahydropyrido[1,2- a]pyrazine-1,4-dione | [M + H]⁺: 373, 375 (3:2); ¹H NMR (400 MHz, CD₃OD) δ 7.20 (d, J = 8.7 Hz, 1H), 6.71 (d, J = 8.8 Hz, 1H), 4.76-4.68 (m, 1H), 4.09 (dd, J = 12.2, 2.8 Hz, 1H), 4.04- 3.99 (m, 1H), 3.99-3.90 (m, 2H), 3.86- 3.68 (m, 1H), 3.01 (s, 3H), 2.92-2.73 (m, 2H), 2.57-2.42 (m, 1H), 2.11 (d, J = 12.7 Hz, 1H), 1.60 (d, J = 13.3 Hz, 1H). |
| 14 | 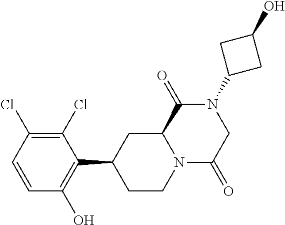 | 8-(2,3-dichloro-6- hydroxyphenyl)-2-[3- hydroxycyclobutyl]- hexahydropyrido[1,2- a]pyrazine-1,4-dione isomer 1 | [M + H]⁺_{:} 399, 401 (3:2); ¹H NMR (400 MHz, CD₃OD) δ 7.21 (d, J = 8.8 Hz, 1H), 6.72 (d, J = 8.8 Hz, 1H), 4.74-4.65 (m, 1H), 4.35-4.21 (m, 1H), 4.17-4.04 (m, 3H), 4.04-3.95 (m, 1H), 3.85-3.72 (m, 1H), 2.79 (td, J = 13.1, 3.0 Hz, 1H), 2.65- 2.53 (m, 2H), 2.53-2.40 (m, 2H), 2.19- 2.06 (m, 3H), 1.69-1.60 (m, 1H). |
| 15 | 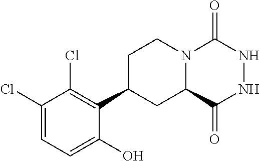 [SMILES: O=C1NNC(=O)N2CC[C@H](c3c(O)ccc(Cl)c3Cl)C[C@H]12] | (8S,9aR)-8-(2,3- dichloro-6- hydroxyphenyl)hexahydro- 4H-pyrido[1,2- d][1,2,4]triazine- 1,4(6H)-dione | [M + H]⁺: 330, 332 (3:2); ¹H NMR (400 MHz, DMSO-d6) δ 12.15 (brs, 1H), 10.13 (s, 1H), 7.29 (d, J = 8.8 Hz, 1H), 6.81 (d, J = 8.8 Hz, 1H), 3.66 (dd, J = 11.5, 2.7 Hz, 1H), 3.50-3.36 (m, 1H), 3.13-3.01 (m, 1H), 2.73-2.56 (m, 2H), 2.49-2.42 (m, 1H), 2.37-2.23 (m, 1H), 1.70-1.59 (m, 1H), 1.44-1.33 (m, 1H). |
| 16 | 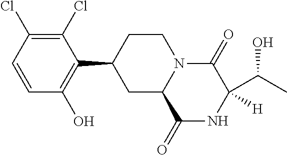 [SMILES: C[C@@H](O)[C@H]1NC(=O)[C@H]2C[C@@H](c3c(O)ccc(Cl)c3Cl)CCN2C1=O] | (3R,8S,9aR)-8-(2,3- dichloro-6- hydroxyphenyl)-3- [(1R)-1- hydroxyethyl]- hexahydro-2H- pyrido[1,2- a]pyrazine-1,4-dione | [M + H]⁺ 373, 375 (3:2); ¹H NMR (400 MHz, CD₃OD) δ 7.21 (d, J = 8.7 Hz, 1H), 6.70 (d, J = 8.7 Hz, 1H), 4.77-4.64 (m, 1H), 4.33-4.21 (m, 1H), 4.19-4.05 (m, 2H), 3.90-3.71 (m, 1H), 2.82-2.57 (m, 2H), 2.52-2.35 (m, 1H), 2.23-2.11 (m, 1H), 1.63 (d, J = 13.3 Hz, 1H), 1.22 (d, J = 6.4 Hz, 3H). |
| 17 | 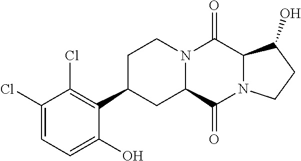 [SMILES: O=C1[C@H]2[C@H](O)CCN2C(=O)[C@H]2C[C@@H](c3c(O)ccc(Cl)c3Cl)CCN12] | 7-(2,3-dichloro-6- hydroxyphenyl)-1- hydroxyhexahydro- 1H-pyrido[1,2- a]pyrrolo[1,2- d]pyrazine- 5,11(5aH,11aH)- dione isomer 2 | [M + H]⁺: 385, 387 (3:2); ¹H NMR (400 MHz, CD₃OD) δ 7.22 (d, J = 8.8 Hz, 1H), 6.73 (d, J = 8.8 Hz, 1H), 4.69-4.59 (m, 1H), 4.40 (q, J = 7.5 Hz, 1H), 4.15-4.03 (m, 1H), 3.94 (d, J = 7.4 Hz, 1H), 3.88- 3.68 (m, 2H), 3.54-3.42 (m, 1H), 2.83 (td, J = 13.0, 3.0 Hz, 1H), 2.62 (q, J = 12.5 Hz, 1H), 2.54-2.40 (m, 1H), 2.34-2.21 (m, 1H), 2.07-1.96 (m, 1H), 1.95-1.81 (m, 1H), 1.66-1.56 (m, 1H). |
| 18 | 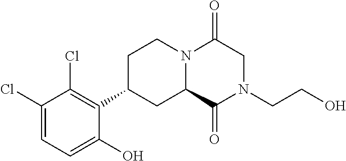 [SMILES: O=C1[C@H]2C[C@H](c3c(O)ccc(Cl)c3Cl)CCN2C(=O)CN1CCO] | (8R,9aR)-rel-8-(2,3- dichloro-6- hydroxyphenyl)-2-(2- hydroxyethyl)- hexahydropyrido[1,2- a]pyrazine-1,4-dione | [M + H]⁺: 373, 375 (3:2); ¹H NMR (400 MHz, CD₃OD) δ 7.23 (d, J = 8.8 Hz, 1H), 6.78 (d, J = 8.8 Hz, 1H), 4.72- 4.66 (m, 1H), 4.31-4.09 (m, 2H), 3.89- 3.67 (m, 4H), 3.68-3.46 (m, 3H), 2.77- 2.67 (m, 1H), 2.62-2.48 (m, 1H), 2.26- 2.14 (m, 1H), 1.82-1.69 (m, 1H). |
| 19 | 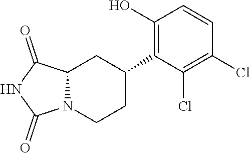 [SMILES: O=C1NC(=O)N2CC[C@@H](c3c(O)ccc(Cl)c3Cl)C[C@@H]12] | 7-(2,3-dichloro-6- hydroxyphenyl)tetrahydro- imidazo[1,5- a]pyridine- 1,3(2H,5H)-dione diastereoisomer 2 | [M + H]⁺: 315, 317 (3:2); ¹H NMR (400 MHz, CD₃OD) δ 7.24 (d, J = 8.8 Hz, 1H), 6.79 (d, J = 8.7 Hz, 1H), 4.62 (dd, J = 10.4, 6.4 Hz, 1H), 3.78-3.66 (m, 1H), 3.62-3.51 (m, 2H), 2.56-2.42 (m, 2H), 2.11-2.00 (m, 1H), 1.82-1.72 (m, 1H). |
| 20 | 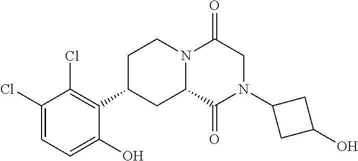 [SMILES: O=C1[C@@H]2C[C@H](c3c(O)ccc(Cl)c3Cl)CCN2C(=O)CN1C1CC(O)C1] | (8R,9aS)-rel-8-(2,3- dichloro-6- hydroxyphenyl)-2-(3- hydroxycyclobutyl)- hexahydropyrido[1,2- a]pyrazine-1,4-dione | [M + H]⁺: 399, 401 (3:2); ¹H NMR (400 MHz, CD₃OD) δ 7.22 (d, J = 8.8 Hz, 1H), 6.72 (d, J = 8.8 Hz, 1H), 4.74-4.66 (m, 1H), 4.33-4.23 (m, 1H), 4.18-4.04 (m, 3H), 4.04-3.95 (m, 1H), 3.85-3.71 (m, 1H), 2.79 (td, J = 13.1, 3.0 Hz, 1H), 2.66- 2.53 (m, 2H), 2.53-2.39 (m, 2H), 2.20- 2.05 (m, 3H), 1.64 (d, J = 13.2 Hz, 1H). |
| 21 | 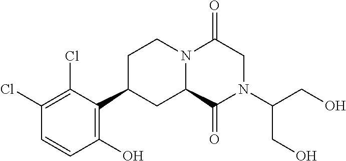 [SMILES: O=C1[C@H]2C[C@@H](c3c(O)ccc(Cl)c3Cl)CCN2C(=O)CN1C(CO)CO] | (8S,9aR)-8-(2,3- dichloro-6- hydroxyphenyl)-2- (1,3- dihydroxypropan-2- yl)hexahydro-4H- pyrido[1,2- a]pyrazine-1,4(6H)- dione | [M + H]⁺: 403, 405 (3:2); ¹H NMR (400 MHz, CD₃OD) δ 7.22 (d, J = 8.8 Hz, 1H), 6.72 (d, J = 8.8 Hz, 1H), 4.75-4.64 (m, 1H), 4.51-4.32 (m, 1H), 4.21 (s, 2H), 4.18-4.10 (m, 1H), 3.91-3.69 (m, 5H), 2.81 (td, J = 13.1, 3.1Hz, 1H), 2.64-2.41 (m, 2H), 2.25-2.13 (m, 1H), 1.70-1.60 (m, 1H). |
| 22 | 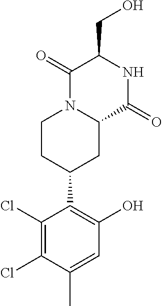 [SMILES: Cc1cc(O)c([C@@H]2CCN3C(=O)[C@@H](CO)NC(=O)[C@@H]3C2)c(Cl)c1Cl] | (8R,9aS)-rel-8-(2,3- dichloro-6-hydroxy- 4-methylphenyl)-3- ((3R)- hydroxymethyl)- hexahydro-2H- pyrido[1,2- a]pyrazine-1,4-dione | [M + H]⁺: 373, 375 (3:2); ¹H NMR (400 MHz, CD₃OD) δ 6.68 (d, J = 4.8 Hz, 1H), 4.78-4.66 (m, 1H), 4.13-3.93 (m, 3H), 3.79-3.63 (m, 2H), 2.85-2.38 (m, 3H), 2.31 (d, J = 3.1 Hz, 3H), 2.26-2.08 (m, 1H), 1.62 (t, J = 15.1 Hz, 1H). |
| 23 | 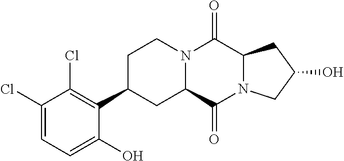 [SMILES: O=C1[C@H]2C[C@H](O)CN2C(=O)[C@H]2C[C@@H](c3c(O)ccc(Cl)c3Cl)CCN12] | 7-(2,3-dichloro-6- hydroxyphenyl)-2- hydroxyhexahydro- 1H-pyrido[1,2- a]pyrrolo[1,2- d]pyrazine- 5,11(5aH,11aH)- dione isomer 1 | [M + H]⁺: 385, 387 (3:2); ¹H NMR (400 MHz, CD₃OD) δ 7.22 (d, J = 8.8 Hz, 1H), 6.77 (d, J = 8.7 Hz, 1H), 4.77-4.69 (m, 1H), 4.58-4.45 (m, 2H), 3.95-3.80 (m, 1H), 3.80-3.64 (m, 3H), 3.55-3.35 (m, 1H), 2.90-2.74 (m, 1H), 2.56-1.99 (m, 4H), 1.93-1.79 (m, 1H). |
| 24 | 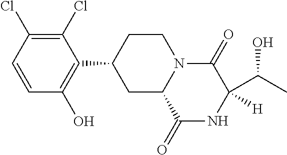 [SMILES: C[C@@H](O)[C@H]1NC(=O)[C@@H]2C[C@H](c3c(O)ccc(Cl)c3Cl)CCN2C1=O] | (3R,8R,9aS)-8-(2,3- dichloro-6- hydroxyphenyl)-3- [(1R)-1- hydroxyethyl]- hexahydro-2H- pyrido[1,2- a]pyrazine-1,4-dione | [M + H]⁺: 373, 375 (3:2); ¹H NMR (400 MHz, CD₃OD) δ 7.22 (d, J = 8.8 Hz, 1H), 6.72 (d, J = 8.8 Hz, 1H), 4.72 (d, J = 13.4 Hz, 1H), 4.28-4.16 (m, 1H), 4.16- 4.04 (m, 2H), 3.87-3.69 (m, 1H), 2.77 (t, J = 12.9 Hz, 1H), 2.59-2.38 (m, 2H), 2.20 (d, J = 13.2 Hz, 1H), 1.65 (d, J = 13.2 Hz, 1H), 1.19 (d, J = 6.4 Hz, 3H). |
| 25 | 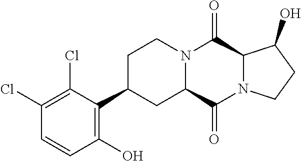 [SMILES: O=C1[C@H]2[C@@H](O)CCN2C(=O)[C@H]2C[C@@H](c3c(O)ccc(Cl)c3Cl)CCN12] | (1S,5aR,7S,11aR)-7- (2,3-dichloro-6- hydroxyphenyl)-1- hydroxyhexahydro- 1H-pyrido[1,2- a]pyrrolo[1,2- d]pyrazine- 5,11(5aH,11aH)- dione | [M + H]⁺_{:} 385, 387 (3:2); ¹H NMR (400 MHz, DMSO-d₆) δ 10.12 (s, 1H), 7.30 (d, J = 8.8 Hz, 1H), 6.81 (d, J = 8.8 Hz, 1H), 5.22 (d, J = 4.4 Hz, 1H), 4.61-4.51 (m, 1H), 4.43-4.38 (m, 1H), 4.10-4.01 (m, 2H), 3.81-3.69 (m, 1H), 3.53 (brs, 1H), 3.31-3.20 (m, 1H), 2.70-2.59 (m, 1H), 2.35-2.17 (m, 2H), 2.13-2.04 (m, 1H), 2.04-1.89 (m, 1H), 1.82-1.72 (m, 1H), 1.62-1.50 (m, 1H). |
| 26 | 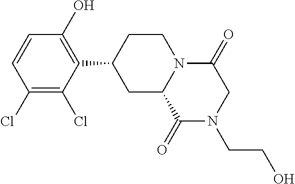 [SMILES: O=C1[C@@H]2C[C@H](c3c(O)ccc(Cl)c3Cl)CCN2C(=O)CN1CCO] | (8R,9aS)-8-(2,3- dichloro-6- hydroxyphenyl)-2-(2- hydroxyethyl)- hexahydropyrido[1,2- a]pyrazine-1,4-dione | [M + H]⁺: 373, 375 (3:2); ¹H NMR (400 MHz, CD₃OD) δ 7.22 (d, J = 8.8 Hz, 1H), 6.73 (d, J = 8.8 Hz, 1H), 4.76-4.66 (m, 1H), 4.34-4.16 (m, 2H), 4.16-4.06 (m, 1H), 3.87-3.71 (m, 3H), 3.59-3.46 (m, 2H), 2.80 (td, J = 13.1, 3.0 Hz, 1H), 2.62- 2.41 (m, 2H), 2.23-2.12 (m, 1H), 1.64 (d, J = 13.2 Hz, 1H). |
| 27 | 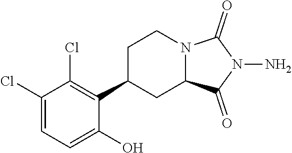 [SMILES: NN1C(=O)[C@H]2C[C@@H](c3c(O)ccc(Cl)c3Cl)CCN2C1=O] | (7S,8aR)-2-amino-7- (2,3-dichloro-6- hydroxyphenyl)tetrahydro- imidazo[1,5- a]pyridine- 1,3(2H,5H)-dione | [M + H]⁺: 330, 332 (3:2); ¹H NMR (400 MHz, CD₃OD) δ 7.22 (d, J = 8.7 Hz, 1H), 6.72 (d, J = 8.8 Hz, 1H), 4.21 (dd, J = 13.4, 5.1 Hz, 1H), 4.10 (dd, J = 12.1, 4.3 Hz, 1H), 3.79-3.64 (m, 1H), 3.09 (td, J = 13.2, 3.5 Hz, 1H), 2.51-2.34 (m, 2H), 2.08-1.98 (m, 1H), 1.64 (d, J = 13.2 Hz, 1H). |
| 28 | 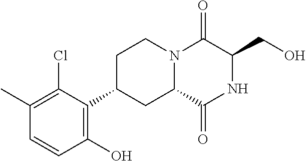 [SMILES: Cc1ccc(O)c([C@@H]2CCN3C(=O)[C@@H](CO)NC(=O)[C@@H]3C2)c1Cl] | (8R,9aS)-rel-8-(2- chloro-6-hydroxy-3- methylphenyl)-3- ((3R)- hydroxymethyl)- hexahydro-2H- pyrido[1,2- a]pyrazine-1,4-dione | [M + H]⁺: 339, 341 (3:1); ¹H NMR (400 MHz, CD₃OD) δ 7.01-6.92 (m, 1H), 6.66-6.57 (m, 1H), 4.79-4.67 (m, 1H), 4.13-3.92 (m, 3H), 3.84-3.68 (m, 2H), 2.82-2.44 (m, 3H), 2.28 (d, J = 3.6 Hz, 3H), 2.25-2.09 (m, 1H), 1.61 (t, J = 14.1 Hz, 1H). |
| 29 | 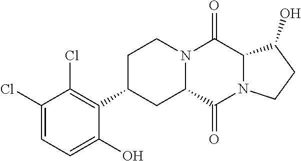 [SMILES: O=C1[C@@H]2C[C@H](c3c(O)ccc(Cl)c3Cl)CCN2C(=O)[C@@H]2[C@H](O)CCN12] | 7-(2,3-dichloro-6- hydroxyphenyl)-1- hydroxyhexahydro- 1H-pyrido[1,2- a]pyrrolo[1,2- d]pyrazine- 5,11(5aH,11aH)- dione isomer 1 | [M + H]⁺: 385, 387 (3:2); ¹H NMR (400 MHz, CD₃OD) δ 7.22 (d, J = 8.8 Hz, 1H), 6.72 (d, J = 8.8 Hz, 1H), 4.71-4.63 (m, 1H), 4.42 (q, J = 7.5 Hz, 1H), 4.19-4.12 (m, 1H), 3.95-3.89 (m, 1H), 3.78-3.63 (m, 2H), 3.55-3.43 (m, 1H), 2.84 (td, J = 13.2, 3.3 Hz, 1H), 2.52-2.38 (m, 2H), 2.33-2.19 (m, 2H), 1.93-1.81 (m, 1H), 1.74-1.65 (m, 1H). |
| 30 | 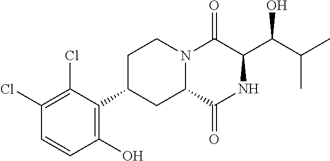 [SMILES: CC(C)[C@H](O)[C@H]1NC(=O)[C@@H]2C[C@H](c3c(O)ccc(Cl)c3Cl)CCN2C1=O] | (3R,8R.9aS)-8-(2,3- dichloro-6- hydroxyphenyl)-3- [(1S)-1-hydroxy-2- methylpropyl]- hexahydro-2H- pyrido[1,2- a]pyrazine-1,4-dione | [M + H]⁺: 401, 403 (3:2); ¹H NMR (400 MHz, CD₃OD) δ 7.21 (d, J = 8.8 Hz, 1H), 6.72 (d, J = 8.8 Hz, 1H), 4.79-4.70 (m, 1H), 4.16-4.11 (m, 1H), 4.06 (dd, J = 12.2, 3.4 Hz, 1H), 3.80-3.64 (m, 1H), 3.58 (dd, J = 10.1, 1.5 Hz, 1H), 2.76 (td, J = 13.3, 3.1 Hz, 1H), 2.55-2.42 (m, 2H), 2.28-2.19 (m, 1H), 1.88-1.73 (m, 1H), 1.66 (d, J = 13.2 Hz, 1H), 1.06 (d, J = 6.5 Hz, 3H), 0.97 (d, J = 6.6 Hz, 3H). |
| 31 | 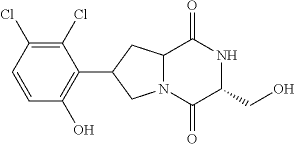 [SMILES: O=C1N[C@H](CO)C(=O)N2CC(c3c(O)ccc(Cl)c3Cl)CC12] | (3R)-7-(2,3-dichloro- 6-hydroxyphenyl)-3- (hydroxymethyl)hexahydro- pyrrolo[1,2- a]pyrazine-1,4-dione | [M + H]⁺: 345, 347 (3:2); ¹H NMR (400 MHz, DMSO-d₆) δ 10.38 (s, 1H), 8.16 (d, J = 3.6 Hz, 1H), 7.36 (d, J = 8.8 Hz, 1H), 6.83 (d, J = 8.9 Hz, 1H), 5.29 (t, J = 5.4 Hz, 1H), 4.39 (dd, J = 11.3, 6.4 Hz, 1H), 4.21-4.08 (m, 1H), 3.94-3.86 (m, 1H), 3.80-3.69 (m, 2H), 3.63-3.43 (m, 2H), 2.46-2.38 (m, 1H), 2.28-2.14 (m, 1H). |
| 32 | 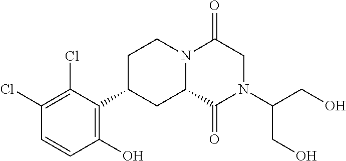 [SMILES: O=C1[C@@H]2C[C@H](c3c(O)ccc(Cl)c3Cl)CCN2C(=O)CN1C(CO)CO] | (8R,9aS)-8-(2,3- dichloro-6- hydroxyphenyl)-2- (1,3- dihydroxypropan-2- yl)hexahydro-4H- pyrido[1,2- a]pyrazine-1,4(6H)- dione | [M + H]⁺: 403, 405 (3:2); ¹H NMR (400 MHz, CD₃OD) δ 7.22 (d, J = 8.8 Hz, 1H), 6.72 (d, J = 8.8 Hz, 1H), 4.74-4.66 (m, 1H), 4.50-4.40 (m, 1H), 4.21 (s, 2H), 4.17-4.11 (m, 1H), 3.85-3.71 (m, 5H), 2.81 (td, J = 13.2, 3.0 Hz, 1H), 2.64-2.43 (m, 2H), 2.23-2.13 (m, 1H), 1.67-1.57 (m, 1H). |
| 33 | 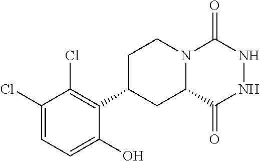 [SMILES: O=C1NNC(=O)N2CC[C@@H](c3c(O)ccc(Cl)c3Cl)C[C@@H]12] | (8R,9aS)-8-(2,3- dichloro-6- hydroxyphenyl)hexahydro- 4H-pyrido[1,2- d][1,2,4]triazine- 1,4(6H)-dione | [M + H]⁺: 330, 332 (3:2); ¹H NMR (400 MHz, DMSO-d₆) δ 12.14 (brs, 1H), 10.12 (s, 1H), 7.29 (d, J = 8.8 Hz, 1H), 6.81 (d, J = 8.8 Hz, 1H), 3.66 (dd, J = 11.4, 2.7 Hz, 1H), 3.48-3.36 (m, 1H), 3.08 (d, J = 11.8 Hz, 1H), 2.73-2.56 (m, 2H), 2.49- 2.42 (m, 1H), 2.36-2.22 (m, 1H), 1.64 (d, J = 12.3 Hz, 1H), 1.44-1.34 (m, 1H). |
| 34 | 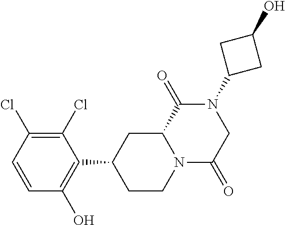 | 8-(2,3-dichloro-6- hydroxyphenyl)-2-[3- hydroxycyclobutyl]- hexahydropyrido[1,2- a]pyrazine-1,4-dione isomer 3 | [M + H]⁺_{:} 399, 401 (3:2); ¹H NMR (400 MHz, CD₃OD) δ 7.21 (d, J = 8.8 Hz, 1H), 6.72 (d, J = 8.8 Hz, 1H), 4.74-4.61 (m, 1H), 4.34-4.21 (m, 1H), 4.17-4.04 (m, 3H), 4.04-3.95 (m, 1H), 3.87-3.70 (m, 1H), 2.79 (td, J = 13.1, 3.0 Hz, 1H), 2.65- 2.54 (m, 2H), 2.54-2.39 (m, 2H), 2.20- 2.04 (m, 3H), 1.68-1.59 (m, 1H). |
| 35 | 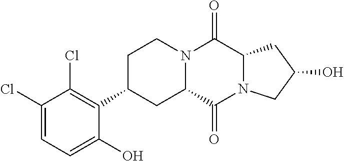 [SMILES: O=C1[C@@H]2C[C@H](O)CN2C(=O)[C@@H]2C[C@H](c3c(O)ccc(Cl)c3Cl)CCN12] | 7-(2,3-dichloro-6- hydroxyphenyl)-2- hydroxyhexahydro- 1H-pyrido[1,2- a]pyrrolo[1,2- d]pyrazine- 5,11(5aH,11aH)- dione isomer 4 | [M + H]⁺: 385, 387 (3:2); ¹H NMR (400 MHz, CD₃OD) δ 7.22 (d, J = 8.8 Hz, 1H), 6.73 (d, J = 8.8 Hz, 1H), 4.69-4.61 (m, 1H), 4.56-4.47 (m, 2H), 4.19-4.08 (m, 1H), 3.99-3.75 (m, 2H), 3.38 (d, J = 13.2 Hz, 1H), 2.86 (td, J = 13.0, 3.0 Hz, 1H), 2.66 (q, J = 12.5 Hz, 1H), 2.55-2.33 (m, 2H), 2.12-1.99 (m, 2H), 1.68-1.57 (m, 1H). |
| 36 | 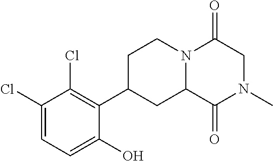 [SMILES: CN1CC(=O)N2CCC(c3c(O)ccc(Cl)c3Cl)CC2C1=O] | 8-(2,3-dichloro-6- hydroxyphenyl)-2- methyl-octahydro- 1H-pyrido[1,2- a]pyrazine-1,4-dione | [M + H]⁺: 343, 345 (3:2); ¹H NMR (400 MHz, CD₃OD) δ 7.22 (d, J = 8.8 Hz, 1H), 6.72 (d, J = 8.8 Hz, 1H), 4.74-4.66 (m, 1H), 4.20-4.02 (m, 3H), 3.84-3.70 (m, 1H), 2.98 (s, 3H), 2.79 (td, J = 13.1, 3.1 Hz, 1H), 2.59-2.41 (m, 2H), 2.20-2.13 (m, 1H), 1.68-1.58 (m, 1H). |
| 37 | 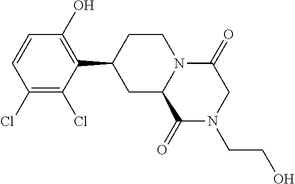 [SMILES: O=C1[C@H]2C[C@@H](c3c(O)ccc(Cl)c3Cl)CCN2C(=O)CN1CCO] | (8S,9aR)-8-(2,3- dichloro-6- hydroxyphenyl)-2-(2- hydroxyethyl)- hexahydropyrido[1,2- a]pyrazine-1,4-dione | [M + H]⁺: 373, 375 (3:2); ¹H NMR (400 MHz, CD₃OD) δ 7.22 (d, J = 8.8 Hz, 1H), 6.73 (d, J = 8.8 Hz, 1H), 4.74-4.66 (m, 1H), 4.34-4.15 (m, 2H), 4.15-4.07 (m, 1H), 3.86-3.68 (m, 3H), 3.60-3.46 (m, 2H), 2.80 (td, J = 13.0, 3.0 Hz, 1H), 2.62- 2.40 (m, 2H), 2.23-2.11 (m, 1H), 1.69- 1.59 (m, 1H). |
| 38 | 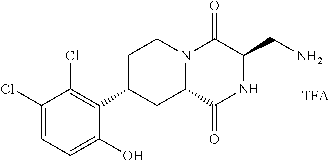 [SMILES: NC[C@H]1NC(=O)[C@@H]2C[C@H](c3c(O)ccc(Cl)c3Cl)CCN2C1=O] | (8R,9aS)-rel-3-((3R)- aminomethyl)-8-(2,3- dichloro-6- hydroxyphenyl)- hexahydro-2H- pyrido[1,2- a]pyrazine-1,4-dione; trifluoroacetic acid | [M + H]⁺: 358, 360 (3:2); ¹H NMR (400 MHz, CD₃OD) δ 7.23 (d, J = 8.7 Hz, 1H), 6.74 (d, J = 8.9 Hz, 1H), 4.81-4.71 (m, 1H), 4.50-4.32 (m, 1H), 4.19-4.07 (m, 1H), 3.86-3.72 (m, 1H), 3.49-3.35 (m, 1H), 3.31-3.24 (m, 1H), 2.81 (td, J = 13.2, 3.0 Hz, 1H), 2.67-2.40 (m, 2H), 2.29-2.10 (m, 1H), 1.74-1.57 (m, 1H); ¹⁹F NMR (400 MHz, CD₃OD) δ −77.03. |
| 39 | 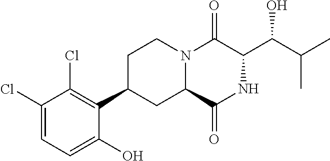 [SMILES: CC(C)[C@@H](O)[C@@H]1NC(=O)[C@H]2C[C@@H](c3c(O)ccc(Cl)c3Cl)CCN2C1=O] | (3S,8S,9aR)-8-(2,3- dichloro-6- hydroxyphenyl)-3- [(1R)-1-hydroxy-2- methylpropyl]- hexahydro-2H- pyrido[1,2- a]pyrazine-1,4-dione | [M + H]⁺: 401, 403 (3:2); ¹H NMR (400 MHz, CD₃OD) δ 7.18 (d, J = 8.7 Hz, 1H), 6.70 (d, J = 8.8 Hz, 1H), 4.77-4.68 (m, 1H), 4.11 (s, 1H), 4.06-3.98 (m, 1H), 3.85-3.65 (m, 1H), 3.57 (dd, J = 10.0, 1.7 Hz, 1H), 2.92-2.66 (m, 2H), 2.56-2.42 (m, 1H), 2.09 (d, J = 12.7 Hz, 1H), 1.85- 1.71 (m, 1H), 1.62-1.54 (m, 1H), 1.04 (d, J = 6.5 Hz, 3H), 0.95 (d, J = 6.6 Hz, 3H). |
| 40 | 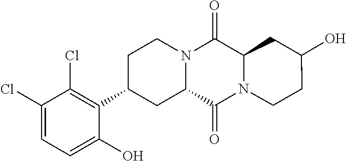 | (2R,6aR,12aS)-2- (2,3-dichloro-6- hydroxyphenyl)-8- hydroxyoctahydro- dipyrido[1,2-a:1′,2′- d]pyrazine- 6,12(2H,6aH)-dione | [M + H]⁺: 399, 401 (3:2); ¹H NMR (400 MHz, CD₃OD) δ 7.22 (d, J = 8.8 Hz, 1H), 6.72 (d, J = 8.8 Hz, 1H), 4.77-4.68 (m, 1H), 4.68-4.60 (m, 1H), 4.19-4.06 (m, 2H), 3.94-3.72 (m, 2H), 2.79 (td, J = 13.1, 3.0 Hz, 1H), 2.68 (td, J = 13.4, 2.8 Hz, 1H), 2.62-2.55 (m, 1H), 2.55-2.40 (m, 2H), 2.24-2.14 (m, 1H), 2.05-1.95 (m, 1H), 1.64 (d, J = 13.3 Hz, 1H), 1.50-1.28 (m, 2H). |
| 41 | 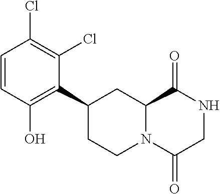 [SMILES: O=C1NCC(=O)N2CC[C@@H](c3c(O)ccc(Cl)c3Cl)C[C@@H]12] | (8R,9aS)-8-(2,3- dichloro-6- hydroxyphenyl)hexahydro- 4H-pyrido[1,2- a]pyrazine-1,4(6H)- dione | [M + H]⁺: 329, 331 (3:2); ¹H NMR (400 MHz, CD₃OD) δ 7.22 (d, J = 8.8 Hz, 1H), 6.73 (d, J = 8.8 Hz, 1H), 4.76-4.65 (m, 1H), 4.16-4.06 (m, 1H), 4.06-3.98 (m, 2H), 3.85-3.69 (m, 1H), 2.79 (td, J = 13.1, 3.0 Hz, 1H), 2.62-2.38 (m, 2H), 2.21-2.11 (m, 1H), 1.64 (d, J = 13.3 Hz, 1H). |
| 42 | 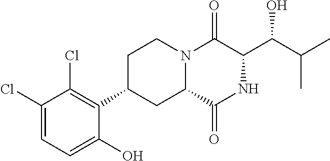 [SMILES: CC(C)[C@@H](O)[C@@H]1NC(=O)[C@@H]2C[C@H](c3c(O)ccc(Cl)c3Cl)CCN2C1=O] | (3S,8R,9aS)-8-(2,3- dichloro-6- hydroxyphenyl)-3- [(1R)-1-hydroxy-2- methylpropyl]- hexahydro-2H- pyrido[1,2- a]pyrazine-1,4-dione | [M + H]⁺: 401, 403 (3:2); ¹H NMR (400 MHz, CD₃OD) δ 7.21 (d, J = 8.8 Hz, 1H), 6.73 (d, J = 8.8 Hz, 1H), 4.78-4.70 (m, 1H), 4.15-4.10 (m, 1H), 4.06 (dd, J = 12.2, 3.4 Hz, 1H), 3.80-3.62 (m, 1H), 3.61-3.55 (m, 1H), 2.76 (td, J = 13.3, 3.1 Hz, 1H), 2.57-2.41 (m, 2H), 2.30-2.20 (m, 1H), 1.86-1.73 (m, 1H), 1.69-1.63 (m, 1H), 1.06 (d, J = 6.5 Hz, 3H), 0.97 (d, J = 6.6 Hz, 3H). |
| 43 | 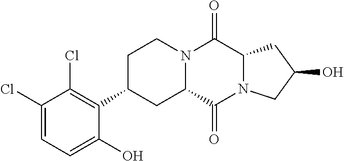 [SMILES: O=C1[C@@H]2C[C@@H](O)CN2C(=O)[C@@H]2C[C@H](c3c(O)ccc(Cl)c3Cl)CCN12] | (2R,5aS,7R,11aS)-7- (2,3-dichloro-6- hydroxyphenyl)-2- hydroxyhexahydro- 1H-pyrido[1,2- a]pyrrolo[1,2- d]pyrazine- 5,11(5aH,11aH)- dione | [M + H]⁺: 385, 387 (3:2); ¹H NMR (400 MHz, CD₃OD) δ 7.21 (d, J = 8.7 Hz, 1H), 6.72 (d, J = 8.8 Hz, 1H), 4.73- 4.62 (m, 1H), 4.55-4.43 (m, 2H), 4.27- 4.17 (m, 1H), 3.90 (dd, J = 13.2, 5.1 Hz, 1H), 3.79-3.61 (m, 1H), 3.40 (d, J = 13.2 Hz, 1H), 2.85 (td, J = 13.1, 3.2 Hz, 1H), 2.56-2.33 (m, 3H), 2.32-2.22 (m, 1H), 2.12-2.01 (m, 1H), 1.75-1.62 (m, 1H). |
| 44 | 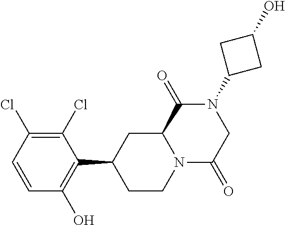 | 8-(2,3-dichloro-6- hydroxyphenyl)-2-[3- hydroxycyclobutyl]- hexahydropyrido[1,2- a]pyrazine-1,4-dione isomer 2 | [M + H]⁺: 399, 401 (3:2); ¹H NMR (400 MHz, CD₃OD) δ 7.21 (d, J = 8.8 Hz, 1H), 6.72 (d, J = 8.8 Hz, 1H), 4.73-4.65 (m, 1H), 4.33-4.21 (m, 1H), 4.17-4.03 (m, 3H), 4.03-3.93 (m, 1H), 3.85-3.70 (m, 1H), 2.79 (td, J = 13.1, 3.0 Hz, 1H), 2.65- 2.54 (m, 2H), 2.53-2.39 (m, 2H), 2.20- 2.02 (m, 3H), 1.67-1.60 (m, 1H). |
| 45 | 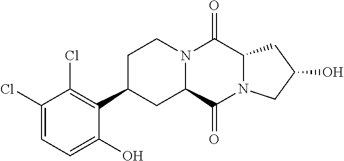 [SMILES: O=C1[C@@H]2C[C@H](O)CN2C(=O)[C@H]2C[C@@H](c3c(O)ccc(Cl)c3Cl)CCN12] | 7-(2,3-dichloro-6- hydroxyphenyl)-2- hydroxyhexahydro- 1H-pyrido[1,2- a]pyrrolo[1,2- d]pyrazine- 5,11(5aH,11aH)- dione isomer 3 | [M + H]⁺: 385, 387 (3:2); ¹H NMR (400 MHz, CD₃OD) δ 7.22 (d, J = 8.8 Hz, 1H), 6.72 (d, J = 8.8 Hz, 1H), 4.76-4.64 (m, 1H), 4.57-4.44 (m, 2H), 4.27-4.18 (m, 1H), 3.90 (dd, J = 13.2, 5.1 Hz, 1H), 3.79- 3.64 (m, 1H), 3.43-3.36 (m, 1H), 2.91- 2.75 (m, 1H), 2.52-2.15 (m, 4H), 2.12- 2.03 (m, 1H), 1.70 (d, J = 13.3 Hz, 1H). |
| 46 | 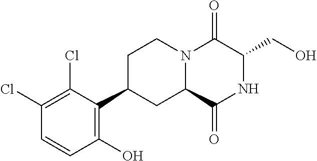 [SMILES: O=C1N[C@@H](CO)C(=O)N2CC[C@H](c3c(O)ccc(Cl)c3Cl)C[C@H]12] | (3S,8S,9aR)-8-(2,3- dichloro-6- hydroxyphenyl)-3- (hydroxymethyl)hexahydro- 4H-pyrido[1,2- a]pyrazine-1,4(6H)- dione | [M + H]⁺: 359, 361 (3:2); ¹H NMR (400 MHz, CD₃OD) δ 7.20 (d, J = 8.8 Hz, 1H), 6.71 (d, J = 8.8 Hz, 1H), 4.77-4.67 (m, 1H), 4.13-4.04 (m, 1H), 4.04-3.95 (m, 2H), 3.87-3.66 (m, 2H), 2.77 (td, J = 13.0, 2.9 Hz, 2H), 2.60-2.38 (m, 1H), 2.20-2.07 (m, 1H), 1.61 (d, J = 13.2 Hz, 1H). |
| 47 | 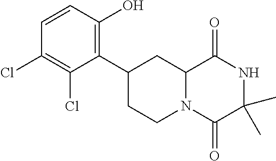 [SMILES: CC1(C)NC(=O)C2CC(c3c(O)ccc(Cl)c3Cl)CCN2C1=O] | 8-(2,3-dichloro-6- hydroxyphenyl)-3,3- dimethyl-octahydro- 1H-pyrido[1,2- a]pyrazine-1,4-dione | [M + H]⁺: 357, 359 (3:2); ¹1H NMR (400 MHz, CD₃OD) δ 7.22 (d, J = 8.8 Hz, 1H), 6.72 (d, J = 8.8 Hz, 1H), 4.71-4.65 (m, 1H), 4.13 (dd, J = 12.4, 3.1 Hz, 1H), 3.88- 3.75 (m, 1H), 2.79 (td, J = 13.1, 2.9 Hz, 1H), 2.64-2.41 (m, 2H), 2.19-2.10 (m, 1H), 1.68-1.60 (m, 1H), 1.53 (s, 3H), 1.50 (s, 3H). |
| 48 | 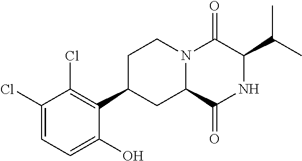 [SMILES: CC(C)[C@H]1NC(=O)[C@H]2C[C@@H](c3c(O)ccc(Cl)c3Cl)CCN2C1=O] | (8S,9aR)-8-(2,3- dichloro-6- hydroxyphenyl)-3- (3R)-isopropyl- hexahydro-2H- pyrido[1,2- a]pyrazine-1,4-dione | [M + H]⁺: 371, 373 (3:2); ¹H NMR (400 MHz, CD₃OD) δ 7.21 (d, J = 8.7 Hz, 1H), 6.70 (d, J = 8.8 Hz, 1H), 4.77-4.69 (m, 1H), 4.14-4.07 (m, 1H), 3.95 (d, J = 3.0 Hz, 1H), 3.86-3.70 (m, 1H), 2.75 (td, J = 13.0, 2.8 Hz, 1H), 2.65 (q, J = 12.5 Hz, 1H), 2.53-2.32 (m, 2H), 2.19-2.11 (m, 1H), 1.64 (d, J = 13.3 Hz, 1H), 1.07 (d, J = 7.1 Hz, 3H), 1.00 (d, J = 6.8 Hz, 3H). |
| 49 | 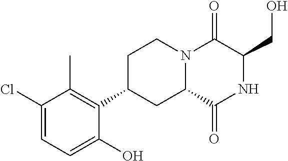 [SMILES: Cc1c(Cl)ccc(O)c1[C@@H]1CCN2C(=O)[C@@H](CO)NC(=O)[C@@H]2C1] | (8R,9aS)-rel-8-(3- chloro-6-hydroxy-2- methylphenyl)-3- ((3R)- hydroxymethyl)- hexahydro-2H- pyrido[1,2- a]pyrazine-1,4-dione | [M + H]⁺: 339, 341 (3:1); ¹H NMR (400 MHz, CD₃OD) δ 7.05 (dd, J = 8.7, 4.8 Hz, 1H), 6.59 (dd, J = 8.7, 3.6 Hz, 1H), 4.77- 4.68 (m, 1H), 4.12-3.94 (m, 3H), 3.78- 3.70 (m, 1H), 3.38-3.34 (m, 1H), 2.83- 2.72 (m, 2H), 2.63-2.45 (m, 1H), 2.43 (d, J = 3.7 Hz, 3H), 2.22-2.07 (m, 1H), 1.60 (t, J = 12.1 Hz, 1H). |
| 50 | 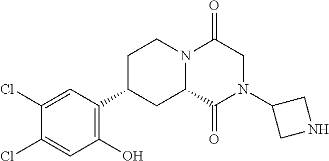 [SMILES: O=C1[C@@H]2C[C@H](c3cc(Cl)c(Cl)cc3O)CCN2C(=O)CN1C1CNC1] | (8R,9aS)-2-(azetidin- 3-yl)-8-(4,5-dichloro- 2- hydroxyphenyl)hexahydro- 4H-pyrido[1,2- a]pyrazine-1,4(6H)- dione | [M + H]⁺: 384, 386 (3:2); ¹H NMR (400 MHz, CD₃OD) δ 7.21 (s, 1H), 6.93 (s, 1H), 5.01-4.88 (m, 1H), 4.77-4.66 (m, 1H), 4.25-4.09 (m, 3H), 3.97-3.87 (m, 2H), 3.80-3.70 (m, 2H), 3.30-3.24 (m, 1H), 2.81 (td, J = 13.0, 2.9 Hz, 1H), 2.49- 2.37 (m, 1H), 1.94-1.85 (m, 1H), 1.80- 1.58 (m, 2H). |
| 51 | 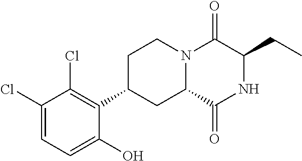 [SMILES: CC[C@H]1NC(=O)[C@@H]2C[C@H](c3c(O)ccc(Cl)c3Cl)CCN2C1=O] | (8R,9aS)-8-(2,3- dichloro-6- hydroxyphenyl)-3- (3R)-ethyl- hexahydro-2H- pyrido[1,2- a]pyrazine-1,4-dione | [M + H]⁺: 357, 359 (3:2); ¹H NMR (400 MHz, CD₃OD) δ 7.21 (d, J = 8.8 Hz, 1H), 6.70 (d, J = 8.8 Hz, 1H), 4.77-4.67 (m, 1H), 4.13-4.05 (m, 2H), 3.87-3.72 (m, 1H), 2.76 (td, J = 13.1, 2.9 Hz, 1H), 2.62 (q, J = 12.5 Hz, 1H), 2.52-2.38 (m, 1H), 2.21-2.11 (m, 1H), 2.09-1.94 (m, 1H), 1.89-1.78 (m, 1H), 1.69-1.57 (m, 1H), 1.02 (t, J = 7.4 Hz, 3H). |
| 52 | 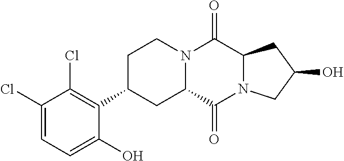 [SMILES: O=C1[C@@H]2C[C@H](c3c(O)ccc(Cl)c3Cl)CCN2C(=O)[C@H]2C[C@@H](O)CN12] | (2R,5aS,7R,11aR)-7- (2,3-dichloro-6- hydroxyphenyl)-2- hydroxyhexahydro- 1H-pyrido[1,2- a]pyrrolo[1,2- d]pyrazine- 5,11(5aH,11aH)- dione | [M + H]⁺: 385, 387 (3:2); ¹H NMR (400 MHz, CD₃OD) δ 7.22 (d, J = 8.8 Hz, 1H), 6.73 (d, J = 8.8 Hz, 1H), 4.69-4.61 (m, 1H), 4.52-4.44 (m, 1H), 4.36 (t, J = 8.5 Hz, 1H), 4.20-4.11 (m, 1H), 3.92-3.78 (m, 1H), 3.78-3.71 (m, 1H), 3.55-3.46 (m, 1H), 2.86 (td, J = 13.0, 3.0 Hz, 1H), 2.72-2.56 (m, 2H), 2.55-2.39 (m, 1H), 2.15-1.97 (m, 2H), 1.62 (d, J = 13.3 Hz, 1H). |
| 53 | 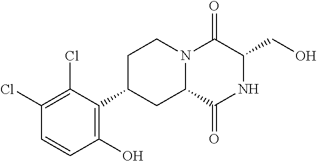 [SMILES: O=C1N[C@@H](CO)C(=O)N2CC[C@@H](c3c(O)ccc(Cl)c3Cl)C[C@@H]12] | (3S,8R,9aS)-8-(2,3- dichloro-6- hydroxyphenyl)-3- (hydroxymethyl)hexahydro- 4H-pyrido[1,2- a]pyrazine-1,4(6H)- dione | [M + H]⁺_{:} 359, 361 (3:2); ¹H NMR (400 MHz, CD₃OD) δ 7.22 (d, J = 8.8 Hz, 1H), 6.72 (d, J = 8.8 Hz, 1H), 4.78-4.69 (m, 1H), 4.13-4.07 (m, 1H), 4.07-3.97 (m, 2H), 3.82-3.69 (m, 2H), 2.78 (td, J = 13.2, 3.0 Hz, 1H), 2.58-2.39 (m, 2H), 2.25-2.16 (m, 1H), 1.70-1.60 (m, 1H). |
| 54 | 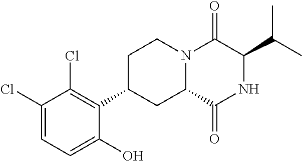 [SMILES: CC(C)[C@H]1NC(=O)[C@@H]2C[C@H](c3c(O)ccc(Cl)c3Cl)CCN2C1=O] | (8R,9aS)-8-(2,3- dichloro-6- hydroxyphenyl)-3- (3R)-isopropyl- hexahydro-2H- pyrido[1,2- a]pyrazine-1,4-dione | [M + H]⁺: 371, 373 (3:2): ¹H NMR (400 MHz, CD₃OD) δ 7.21 (d, J = 8.8 Hz, 1H), 6.72 (d, J = 8.8 Hz, 1H), 4.80-4.66 (m, 1H), 4.12 (dd, J = 12.2, 3.2 Hz, 1H), 3.97- 3.91 (m, 1H), 3.86-3.68 (m, 1H), 2.78 (td, J = 13.2, 3.1 Hz, 1H), 2.59-2.30 (m, 3H), 2.25-2.17 (m, 1H), 1.65 (d, J = 13.2 Hz, 1H), 1.05 (d, J = 7.1 Hz, 3H), 0.92 (d, J= 6.8 Hz, 3H). |
| 55 | 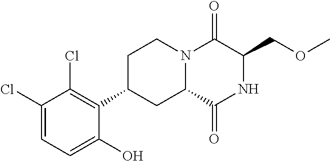 [SMILES: COC[C@H]1NC(=O)[C@@H]2C[C@H](c3c(O)ccc(Cl)c3Cl)CCN2C1=O] | (8R,9aS)-rel-8-(2,3- dichloro-6- hydroxyphenyl)-3- ((3R)- methoxymethyl)- hexahydro-2H- pyrido[1,2- a]pyrazine-1,4-dione | [M + H]⁺: 373, 375 (3:2); ¹H NMR (400 MHz, CD₃OD) δ 7.21 (dd, J = 8.9, 2.3 Hz, 1H), 6.72 (d, J = 8.8 Hz, 1H), 4.76-4.64 (m, 1H), 4.17-4.02 (m, 2H), 3.91-3.66 (m, 2H), 3.63-3.51 (m, 1H), 3.40 (d, J = 17.4 Hz, 3H), 2.88-2.37 (m, 3H), 2.26- 2.04 (m, 1H), 1.73-1.55 (m, 1H). |
| 56 | 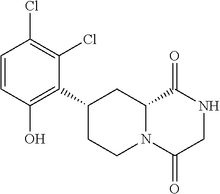 [SMILES: O=C1NCC(=O)N2CC[C@H](c3c(O)ccc(Cl)c3Cl)C[C@H]12] | (8S,9aR)-8-(2,3- dichloro-6- hydroxyphenyl)hexahydro- 4H-pyrido[1,2- a]pyrazine-1,4(6H)- dione | [M + H]⁺: 329, 331 (3:2); ¹H NMR (400 MHz, CD₃OD) δ 7.22 (d, J = 8.8 Hz, 1H), 6.73 (d, J = 8.8 Hz, 1H), 4.76-4.67 (m, 1H), 4.14-4.06 (m, 1H), 4.06-4.00 (m, 2H), 3.88-3.70 (m, 1H), 2.79 (td, J = 13.1, 3.0 Hz, 1H), 2.62-2.40 (m, 2H), 2.22-2.11 (m, 1H), 1.64 (d, J = 13.3 Hz, 1H). |
| 57 | 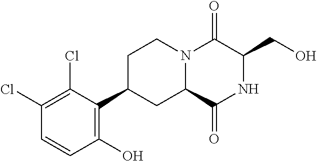 [SMILES: O=C1N[C@H](CO)C(=O)N2CC[C@H](c3c(O)ccc(Cl)c3Cl)C[C@H]12] | (3R,8S,9aR)-8-(2,3- dichloro-6- hydroxyphenyl)-3- (hydroxymethyl)hexahydro- 4H-pyrido[1,2- a]pyrazine-1,4(6H)- dione | [M + H]⁺: 359, 361 (3:2); ¹H NMR (400 MHz, CD₃OD) δ 7.20 (d, J = 8.8 Hz, 1H), 6.71 (d, J = 8.8 Hz, 1H), 4.78-4.70 (m, 1H), 4.08 (dd, J = 12.1, 2.8 Hz, 1H), 4.04- 3.96 (m, 2H), 3.85-3.65 (m, 2H), 2.86- 2.71 (m, 2H), 2.59-2.40 (m, 1H), 2.14 (d, J = 12.7 Hz, 1H), 1.68-1.55 (m, 1H). |
| 58 | 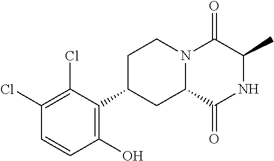 [SMILES: C[C@H]1NC(=O)[C@@H]2C[C@H](c3c(O)ccc(Cl)c3Cl)CCN2C1=O] | (3R,8R,9aS)-8-(2,3- dichloro-6- hydroxyphenyl)-3- methyl-hexahydro- 2H-pyrido[1,2- a]pyrazine-1,4-dione | [M + H]⁺_{:} 343, 345 (3:2); ¹H NMR (400 MHz, CD₃OD) δ 7.22 (d, J = 8.8 Hz, 1H), 6.72 (d, J = 8.8 Hz, 1H), 4.74-4.66 (m, 1H), 4.19-4.07 (m, 2H), 3.87-3.72 (m, 1H), 2.79 (td, J = 13.1, 3.0 Hz, 1H), 2.61- 2.39 (m, 2H), 2.20-2.12 (m, 1H), 1.69- 1.59 (m, 1H), 1.49 (d, J = 6.9 Hz, 3H). |
| 59 | 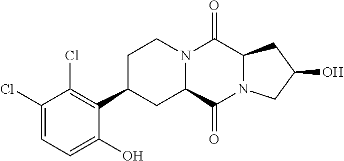 [SMILES: O=C1[C@H]2C[C@@H](O)CN2C(=O)[C@H]2C[C@@H](c3c(O)ccc(Cl)c3Cl)CCN12] | (2R,5aR,7S,11aR)-7- (2,3-dichloro-6- hydroxyphenyl)-2- hydroxyhexahydro- 1H-pyrido[1,2- a]pyrrolo[1,2- d]pyrazine- 5,11(5aH,11aH)- dione | [M + H]⁺: 385, 387 (3:2); ¹H NMR (400 MHz, CD₃OD) δ 7.22 (d, J = 8.8 Hz, 1H), 6.73 (d, J = 8.8 Hz, 1H), 4.65 (d, J = 13.3, 3.6 Hz, 1H), 4.53-4.45 (m, 1H), 4.29 (t, J = 8.6 Hz, 1H), 4.22-4.14 (m, 1H), 3.77- 3.64 (m, 2H), 3.52 (dd, J = 12.5, 6.2 Hz, 1H), 2.86 (td, J = 13.2, 3.3 Hz, 1H), 2.70- 2.61 (m, 1H), 2.60-2.39 (m, 2H), 2.31- 2.22 (m, 1H), 2.17-2.03 (m, 1H), 1.75- 1.66 (m, 1H). |
| 60 | 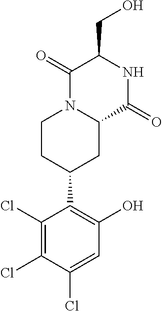 [SMILES: O=C1N[C@H](CO)C(=O)N2CC[C@@H](c3c(O)cc(Cl)c(Cl)c3Cl)C[C@@H]12] | (8R,9aS)- 3-((3R)- hydroxymethyl)-8- (2,3,4-trichloro-6- hydroxyphenyl)- hexahydro-2H- pyrido[1,2- a]pyrazine-1,4-dione | [M + H]⁺: 393, 395, 397 (3:3:1); ¹H NMR (400 MHz, CD₃OD) δ 6.94 (s, 1H), 4.79-4.68 (m, 1H), 4.15-4.07 (m, 1H), 4.07-3.96 (m, 2H), 3.80-3.63 (m, 2H), 2.78 (td, J = 13.2, 3.0 Hz, 1H), 2.54-2.37 (m, 2H), 2.27-2.18 (m, 1H), 1.73-1.61 (m, 1H). |
| 61 | 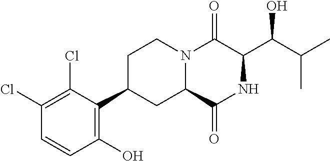 [SMILES: CC(C)[C@H](O)[C@H]1NC(=O)[C@H]2C[C@@H](c3c(O)ccc(Cl)c3Cl)CCN2C1=O] | (3R,8S,9aR)-8-(2,3- dichloro-6- hydroxyphenyl)-3- [(1S)-1-hydroxy-2- methylpropyl]- hexahydro-2H- pyrido[1,2- a]pyrazine-1,4-dione | [M + H]⁺: 401, 403 (3:2); ¹H NMR (400 MHz, CD₃OD) δ 7.20 (d, J = 8.8 Hz, 1H), 6.72 (d, J = 8.8 Hz, 1H), 4.77-4.70 (m, 1H), 4.13 (s, 1H), 4.06 (dd, J = 12.2, 2.8 Hz, 1H), 3.86-3.66 (m, 1H), 3.60 (dd, J = 10.1, 1.7 Hz, 1H), 2.95-2.69 (m, 2H), 2.58-2.45 (m, 1H), 2.12 (d, J = 12.7 Hz, 1H), 1.85-1.71 (m, 1H), 1.61 (d, J = 13.2 Hz, 1H), 1.06 (d, J = 6.5 Hz, 3H), 0.97 (d, J = 6.6 Hz, 3H). |
| 62 | 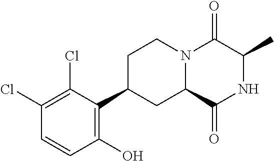 [SMILES: C[C@H]1NC(=O)[C@H]2C[C@@H](c3c(O)ccc(Cl)c3Cl)CCN2C1=O] | (3R,8S,9aR)-8-(2,3- dichloro-6- hydroxyphenyl)-3- methyl-hexahydro- 2H-pyrido[1,2- a]pyrazine-1,4-dione | [M + H]⁺: 343, 345 (3:2); ¹H NMR (400 MHz, CD₃OD) δ 7.22 (d, J = 8.8 Hz, 1H), 6.72 (d, J = 8.8 Hz, 1H), 4.72-4.62 (m, 1H), 4.12-4.04 (m, 2H), 3.87-3.69 (m, 1H), 2.79 (td, J = 13.1, 2.9 Hz, 1H), 2.66- 2.37 (m, 2H), 2.22-2.11 (m, 1H), 1.69- 1.60 (m, 1H), 1.52 (d, J = 7.0 Hz, 3H). |
| 63 | 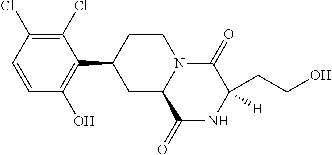 [SMILES: O=C1N[C@H](CCO)C(=O)N2CC[C@H](c3c(O)ccc(Cl)c3Cl)C[C@H]12] | (3R,8S,9aR)-8-(2,3- dichloro-6- hydroxyphenyl)-3-(2- hydroxyethyl)- hexahydro-2H- pyrido[1,2- a]pyrazine-1,4-dione | [M + H]⁺: 373, 375 (3:2); ¹H NMR (400 MHz, CD₃OD) δ 7.22 (d, J = 8.7 Hz, 1H), 6.72 (d, J = 8.8 Hz, 1H), 4.74-4.64 (m, 1H), 4.19-4.13 (m, 1H), 4.13-4.06 (m, 1H), 3.86-3.72 (m, 3H), 2.78 (td, J = 12.9, 2.9 Hz, 1H), 2.60 (q, J = 12.5 Hz, 1H), 2.54-2.40 (m, 1H), 2.21-2.10 (m, 2H), 2.10-1.98 (m, 1H), 1.68-1.59 (m, 1H). |
| 64 | 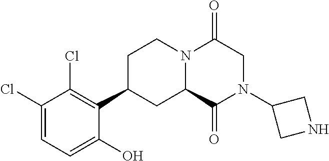 [SMILES: O=C1[C@H]2C[C@@H](c3c(O)ccc(Cl)c3Cl)CCN2C(=O)CN1C1CNC1] | (8S,9aR)-2-(azetidin- 3-yl)-8-(2,3-dichloro- 6-hydroxyphenyl)- octahydro-1H- pyrido[1,2- a]pyrazine-1,4-dione | [M + H]⁺: 384, 386 (3:2); ¹H NMR (400 MHz, CD₃OD) δ 7.22 (d, J = 8.8 Hz, 1H), 6.72 (d, J = 8.8 Hz, 1H), 5.02-4.92 (m, 1H), 4.76-4.63 (m, 1H), 4.30-3.97 (m, 3H), 3.97-3.89 (m, 2H), 3.85-3.65 (m, 3H), 2.79 (td, J = 13.1, 3.1 Hz, 1H), 2.65- 2.40 (m, 2H), 2.21-2.08 (m, 1H), 1.69- 1.56 (m, 1H). |
| 65 | 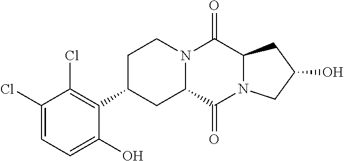 [SMILES: O=C1[C@@H]2C[C@H](c3c(O)ccc(Cl)c3Cl)CCN2C(=O)[C@H]2C[C@H](O)CN12] | 7-(2,3-dichloro-6- hydroxyphenyl)-2- hydroxyhexahydro- 1H-pyrido[1,2- a]pyrrolo[1,2- d]pyrazine- 5,11(5aH,11aH)- dione isomer 2 | [M + H]⁺: 385, 387 (3:2); ¹H NMR (400 MHz, CD₃OD) δ 7.23 (d, J = 8.8 Hz, 1H), 6.78 (d, J = 8.8 Hz, 1H), 4.61-4.55 (m, 2H), 4.48 (t, J = 4.7 Hz, 1H), 4.10-4.00 (m, 1H), 3.92 (dd, J = 13.2, 5.0 Hz, 1H), 3.79-3.67 (m, 1H), 3.47-3.34 (m, 2H), 2.87-2.74 (m, 1H), 2.65-2.56 (m, 1H), 2.38 (dd, J = 13.0, 5.7 Hz, 1H), 2.25-2.14 (m, 1H), 2.09-1.99 (m, 1H), 1.73-1.63 (m, 1H). |
| 66 | 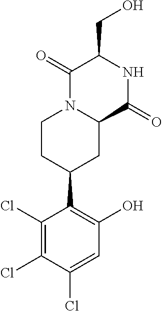 [SMILES: O=C1N[C@H](CO)C(=O)N2CC[C@H](c3c(O)cc(Cl)c(Cl)c3Cl)C[C@H]12] | (8S,9aR)3-((3R)- hydroxymethyl)-8- (2,3,4-trichloro-6- hydroxyphenyl)- hexahydro-2H- pyrido[1,2- a]pyrazine-1,4-dione | [M + H]⁺: 393, 395, 397 (3:3:1); ¹H NMR (400 MHz, CD₃OD) δ 6.93 (s, 1H), 4.78-4.68 (m, 1H), 4.08 (dd, J = 12.2, 2.8 Hz, 1H), 4.04-3.92 (m, 2H), 3.81-3.63 (m, 2H), 2.84-2.71 (m, 2H), 2.55-2.37 (m, 1H), 2.13 (d, J = 12.8 Hz, 1H), 1.61 (d, J = 13.2 Hz, 1H). |
| 67 | 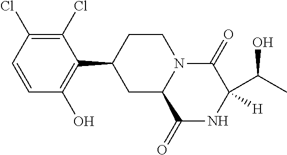 [SMILES: C[C@H](O)[C@H]1NC(=O)[C@H]2C[C@@H](c3c(O)ccc(Cl)c3Cl)CCN2C1=O] | (3R,8S,9aR)-8-(2,3- dichloro-6- hydroxyphenyl)-3- [(1S)-1- hydroxyethyl]- hexahydro-2H- pyrido[1,2- a]pyrazine-1,4-dione | [M + H]⁺: 373, 375 (3:2); ¹H NMR (400 MHz, CD₃OD) δ 7.20 (d, J = 8.8 Hz, 1H), 6.71 (d, J = 8.8 Hz, 1H), 4.78-4.66 (m, 1H), 4.30-4.20 (m, 1H), 4.12-4.03 (m, 1H), 3.94-3.66 (m, 2H), 2.95-2.70 (m, 2H), 2.61-2.44 (m, 1H), 2.11 (d, J = 12.7 Hz, 1H), 1.80-1.51 (m, 1H), 1.27 (d, J = 6.7 Hz, 3H). |
| 68 | 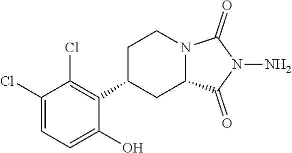 [SMILES: NN1C(=O)[C@@H]2C[C@H](c3c(O)ccc(Cl)c3Cl)CCN2C1=O] | (7R,8aS)-2-amino-7- (2,3-dichloro-6- hydroxyphenyl)tetrahydro- imidazo[1,5- a]pyridine- 1,3(2H,5H)-dione | [M + H]⁺: 330, 332 (3:2); ¹H NMR (400 MHz, CD₃OD) δ 7.22 (d, J = 8.8 Hz, 1H), 6.72 (d, J = 8.8 Hz, 1H), 4.21 (dd, J = 13.3, 5.0 Hz, 1H), 4.10 (dd, J = 12.1, 4.3 Hz, 1H), 3.80-3.65 (m, 1H), 3.09 (td, J = 12.9, 3.5 Hz, 1H), 2.50-2.36 (m, 2H), 2.09-2.00 (m, 1H), 1.64 (d, J = 13.2 Hz, 1H). |
| 69 | 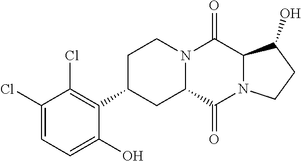 [SMILES: O=C1[C@H]2[C@H](O)CCN2C(=O)[C@@H]2C[C@H](c3c(O)ccc(Cl)c3Cl)CCN12] | 7-(2,3-dichloro-6- hydroxyphenyl)-1- hydroxyhexahydro- 1H-pyrido[1,2- a]pyrrolo[1,2- d]pyrazine- 5,11(5aH,11aH)- dione isomer 3 | [M + H]⁺: 385, 387 (3:2); ¹H NMR (400 MHz, CD₃OD) δ 7.22 (d, J = 8.7 Hz, 1H), 6.73 (d, J = 8.8 Hz, 1H), 4.76-4.68 (m, 1H), 4.65 (t, J = 3.4 Hz, 1H), 4.30-4.27 (m, 1H), 4.17-4.09 (m, 1H), 3.97-3.88 (m, 1H), 3.88-3.75 (m, 1H), 3.55-3.43 (m, 1H), 2.84 (td, J = 13.1, 3.0 Hz, 1H), 2.65-2.40 (m, 2H), 2.23-2.03 (m, 2H), 2.03-1.95 (m, 1H), 1.63 (d, J = 13.4 Hz, 1H). |
| 70 | 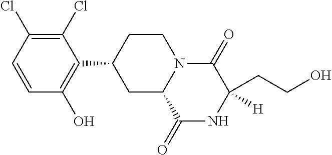 [SMILES: O=C1N[C@H](CCO)C(=O)N2CC[C@@H](c3c(O)ccc(Cl)c3Cl)C[C@@H]12] | (3R,8R,9aS)-8-(2,3- dichloro-6- hydroxyphenyl)-3-(2- hydroxyethyl)- hexahydro-2H- pyrido[1,2- a]pyrazine-1,4-dione | [M + H]⁺: 373, 375 (3:2); ¹H NMR (400 MHz, CD₃OD) δ 7.22 (d, J = 8.7 Hz, 1H), 6.73 (d, J = 8.8 Hz, 1H), 4.77-4.67 (m, 1H), 4.26-4.15 (m, 1H), 4.14-4.05 (m, 1H), 3.88-3.65 (m, 3H), 2.74 (td, J = 13.1, 3.0 Hz, 1H), 2.61-2.41 (m, 2H), 2.22-2.01 (m, 3H), 1.64 (d, J = 13.3 Hz, 1H). |

### Example 9. Evaluation of Kv1.3 Potassium Channel Blocker Activities

This assay is used to evaluate the disclosed compounds' activities as Kv1.3 potassium channel blockers.

### Cell Culture

CHO-K1 cells stably expressing Kv1.3 were grown in DMEM containing 10% heat-inactivated FBS, 1 mM Sodium Pyruvate, 2 mM L-Glutamine and G418 (500 μg/ml). Cells were grown in culture flasks at 37° C. in a 500 CO₂-humidified incubator.

### Solutions

The cells were bathed in an extracellular solution containing 140 mM NaCl, 4 mM KCl, 2 mM CaCl₂, 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The currents were evoked by applying 100 ms depolarizing pulses from −90 mV (holding potential) to +40 mV were applied with 0.1 Hz frequency. Control (compound-free) and compound pulse trains for each compound concentration applied contained 20 pulses. 10-second breaks were used between pulse trains (see Table A below).

### Patch Clamp Recordings and Compound Application

Whole-cell current recordings and compound application were enabled by means of an automated patch clamp platform Patchliner (Nanion Technologies GmbH). EPC 10 patch clamp amplifier (HEKA Elektronik Dr. Schulze GmbH) along with Patchmaster software (HEKA Elektronik Dr. Schulze GmbH) was used for data acquisition. Data were sampled at 10 kHz without filtering. Passive leak currents were subtracted online using a P/4 procedure (HEKA Elektronik Dr. Schulze GmbH). Increasing compound concentrations were applied consecutively to the same cell without washouts in between. Total compound incubation time before the next pulse train was not longer than 10 seconds. Peak current inhibition was observed during compound equilibration.

### Data Analysis

AUC and peak values were obtained with Patchmaster (HEKA Elektronik Dr. Schulze GmbH). To determine IC₅₀, the last single pulse in the pulse train corresponding to a given compound concentration was used. Obtained AUC and peak values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ value is the concentration at which current inhibition is half-maximal, [compound] is the applied compound concentration, A is the fraction of current that is not blocked and nH is the Hill coefficient.

### Example 10. Evaluation of hERG Activities

This assay is used to evaluate the disclosed compounds' inhibition activities against the hERG channel.

hERG Electrophysiology

This assay is used to evaluate the disclosed compounds' inhibition activities against the hERG channel.

### Cell Culture

CHO-K1 cells stably expressing hERG were grown in Ham's F-12 Medium with Glutamine containing 10% heat-inactivated FBS, 1% Penicillin/Streptomycin, Hygromycin (100 μg/ml) and G418 (100 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The voltage protocol (see Table B) was designed to simulate voltage changes during a cardiac action potential with a 300 ms depolarization to +20 mV (analogous to the plateau phase of the cardiac action potential), a repolarization for 300 ms to −50 mV (inducing a tail current) and a final step to the holding potential of −80 mV. The pulse frequency was 0.3 Hz. Control (compound-free) and compound pulse trains for each compound concentration applied contained 70 pulses.

### Patch Clamp Recordings and Compound Application

Whole-cell current recordings and compound application were enabled by means of an automated patch clamp platform Patchliner (Nanion). EPC 10 patch clamp amplifier (HEKA) along with Patchmaster software (HEKA Elektronik Dr. Schulze GmbH) was used for data acquisition. Data were sampled at 10 kHz without filtering. Increasing compound concentrations were applied consecutively to the same cell without washouts in between.

### Data Analysis

AUC and PEAK values were obtained with Patchmaster (HEKA Elektronik Dr. Schulze GmbH). To determine IC₅₀ the last single pulse in the pulse train corresponding to a given compound concentration was used. Obtained AUC and PEAK values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ is the concentration at which current inhibition is half-maximal, [compound] is the applied compound concentration, A is the fraction of current that is not blocked and nH is the Hill coefficient.

Table 1 provides a summary of the inhibition activities of certain selected compounds of the instant invention against Kv1.3 potassium channel and hERG channel.

TABLE 1
IC₅₀ (μM) values of certain exemplified compounds of the instant invention against
Kv1.3 potassium channel and hERG channel

| Compound | | | |
|---|---|---|---|
| Number | Structure | Kv1.3 IC₅₀ | hERG IC₅₀ |
| 1 | 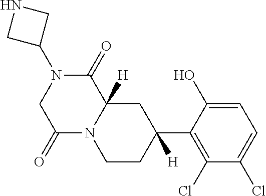 [SMILES: O=C1[C@@H]2C[C@H](c3c(O)ccc(Cl)c3Cl)CCN2C(=O)CN1C1CNC1] | <1 | >30 |
| 2 | 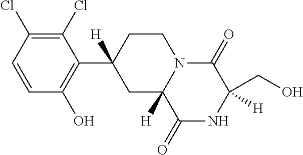 [SMILES: O=C1N[C@H](CO)C(=O)N2CC[C@@H](c3c(O)ccc(Cl)c3Cl)C[C@@H]12] | <1 | >30 |
| 3 | 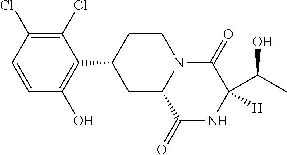 [SMILES: C[C@H](O)[C@H]1NC(=O)[C@@H]2C[C@H](c3c(O)ccc(Cl)c3Cl)CCN2C1=O] | <1 | >30 |
| 4 | 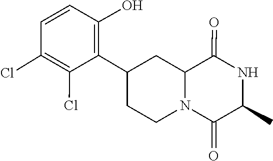 [SMILES: C[C@@H]1NC(=O)C2CC(c3c(O)ccc(Cl)c3Cl)CCN2C1=O] | <10 | * |
| 5 | 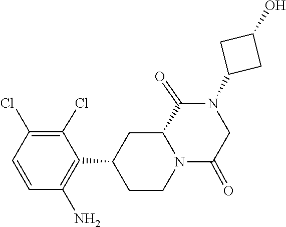 | <10 | * |
| 6 | 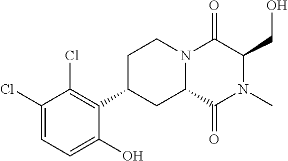 [SMILES: CN1C(=O)[C@@H]2C[C@H](c3c(O)ccc(Cl)c3Cl)CCN2C(=O)[C@H]1CO] | <1 | >30 |
| 7 | 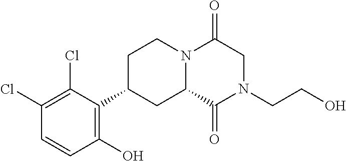 [SMILES: O=C1[C@@H]2C[C@H](c3c(O)ccc(Cl)c3Cl)CCN2C(=O)CN1CCO] | <1 | >30 |
| 8 | 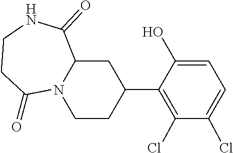 [SMILES: O=C1NCCC(=O)N2CCC(c3c(O)ccc(Cl)c3Cl)CC12] | <1 | >30 |
| 9 | 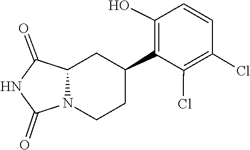 [SMILES: O=C1NC(=O)N2CC[C@H](c3c(O)ccc(Cl)c3Cl)C[C@@H]12] | <1 | >30 |
| 10 | 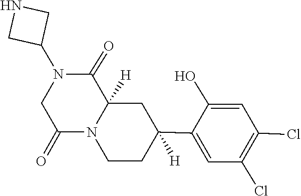 [SMILES: O=C1[C@H]2C[C@@H](c3cc(Cl)c(Cl)cc3O)CCN2C(=O)CN1C1CNC1] | <10 | * |
| 11 | 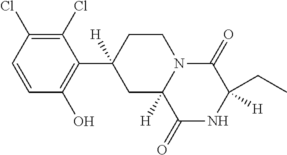 [SMILES: CC[C@H]1NC(=O)[C@H]2C[C@@H](c3c(O)ccc(Cl)c3Cl)CCN2C1=O] | <1 | * |
| 12 | 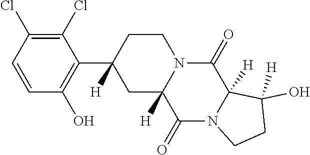 [SMILES: O=C1[C@H]2[C@@H](O)CCN2C(=O)[C@@H]2C[C@H](c3c(O)ccc(Cl)c3Cl)CCN12] | <1 | >30 |
| 13 | 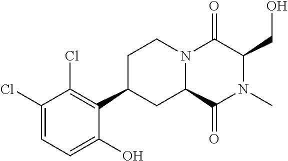 [SMILES: CN1C(=O)[C@H]2C[C@@H](c3c(O)ccc(Cl)c3Cl)CCN2C(=O)[C@H]1CO] | <30 | * |
| 14 | 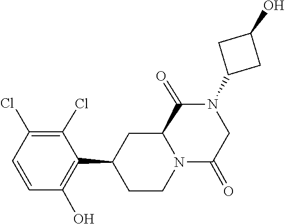 | <1 | >30 |
| 15 | 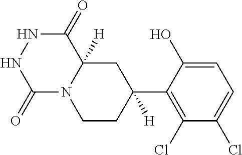 [SMILES: O=C1NNC(=O)N2CC[C@H](c3c(O)ccc(Cl)c3Cl)C[C@H]12] | <10 | * |
| 16 | 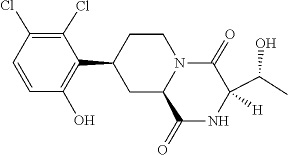 [SMILES: C[C@@H](O)[C@H]1NC(=O)[C@H]2C[C@@H](c3c(O)ccc(Cl)c3Cl)CCN2C1=O] | <10 | * |
| 17 | 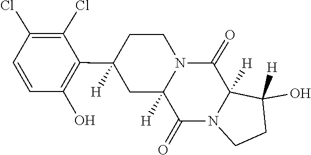 [SMILES: O=C1[C@H]2[C@H](O)CCN2C(=O)[C@H]2C[C@@H](c3c(O)ccc(Cl)c3Cl)CCN12] | <1 | * |
| 18 | 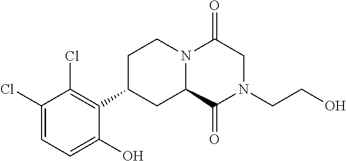 [SMILES: O=C1[C@H]2C[C@H](c3c(O)ccc(Cl)c3Cl)CCN2C(=O)CN1CCO] | <1 | >30 |
| 19 | 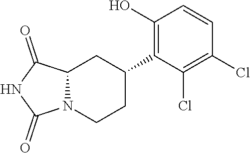 [SMILES: O=C1NC(=O)N2CC[C@@H](c3c(O)ccc(Cl)c3Cl)C[C@@H]12] | <1 | * |
| 20 | 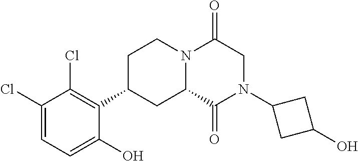 | <1 | >30 |
| 21 | 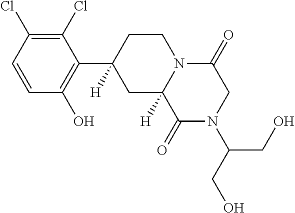 [SMILES: O=C1[C@H]2C[C@@H](c3c(O)ccc(Cl)c3Cl)CCN2C(=O)CN1C(CO)CO] | <10 | * |
| 22 | 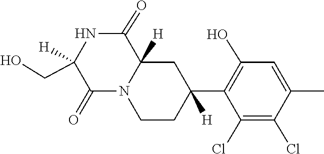 [SMILES: Cc1cc(O)c([C@@H]2CCN3C(=O)[C@@H](CO)NC(=O)[C@@H]3C2)c(Cl)c1Cl] | <1 | >30 |
| 23 | 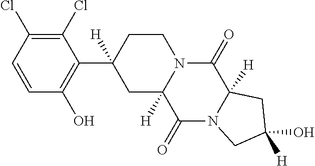 [SMILES: O=C1[C@H]2C[C@H](O)CN2C(=O)[C@H]2C[C@@H](c3c(O)ccc(Cl)c3Cl)CCN12] | <10 | * |
| 24 | 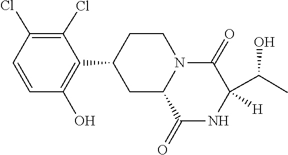 [SMILES: C[C@@H](O)[C@H]1NC(=O)[C@@H]2C[C@H](c3c(O)ccc(Cl)c3Cl)CCN2C1=O] | <1 | >30 |
| 25 | 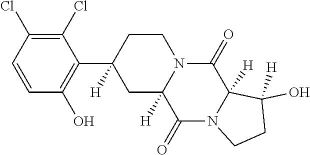 [SMILES: O=C1[C@H]2[C@@H](O)CCN2C(=O)[C@H]2C[C@@H](c3c(O)ccc(Cl)c3Cl)CCN12] | <10 | * |
| 26 | 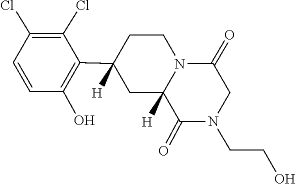 [SMILES: O=C1[C@@H]2C[C@H](c3c(O)ccc(Cl)c3Cl)CCN2C(=O)CN1CCO] | <1 | >30 |
| 27 | 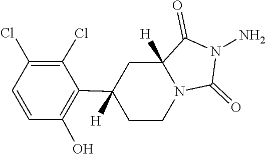 [SMILES: NN1C(=O)[C@H]2C[C@@H](c3c(O)ccc(Cl)c3Cl)CCN2C1=O] | <1 | * |
| 28 | 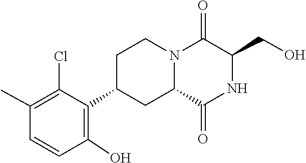 [SMILES: Cc1ccc(O)c([C@@H]2CCN3C(=O)[C@@H](CO)NC(=O)[C@@H]3C2)c1Cl] | <1 | * |
| 29 | 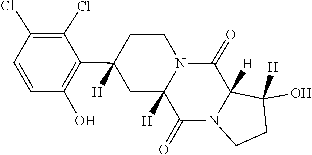 [SMILES: O=C1[C@@H]2C[C@H](c3c(O)ccc(Cl)c3Cl)CCN2C(=O)[C@@H]2[C@H](O)CCN12] | <10 | * |
| 30 | 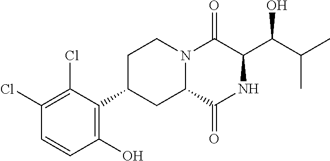 [SMILES: CC(C)[C@H](O)[C@H]1NC(=O)[C@@H]2C[C@H](c3c(O)ccc(Cl)c3Cl)CCN2C1=O] | <1 | >30 |
| 31 | 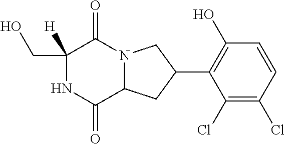 [SMILES: O=C1N[C@H](CO)C(=O)N2CC(c3c(O)ccc(Cl)c3Cl)CC12] | <1 | * |
| 32 | 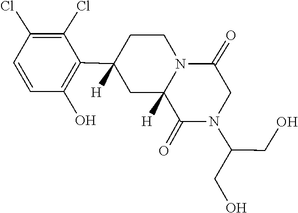 [SMILES: O=C1[C@@H]2C[C@H](c3c(O)ccc(Cl)c3Cl)CCN2C(=O)CN1C(CO)CO] | <1 | >30 |
| 33 | 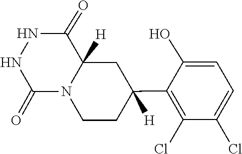 [SMILES: O=C1NNC(=O)N2CC[C@@H](c3c(O)ccc(Cl)c3Cl)C[C@@H]12] | <1 | * |
| 34 | 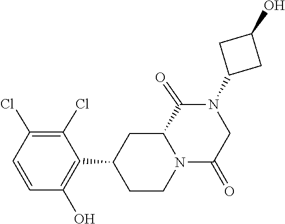 | <10 | * |
| 35 | 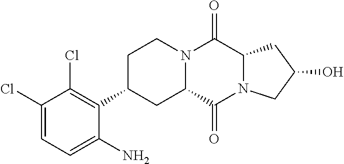 [SMILES: Nc1ccc(Cl)c(Cl)c1[C@@H]1CCN2C(=O)[C@@H]3C[C@H](O)CN3C(=O)[C@@H]2C1] | <1 | <30 |
| 36 | 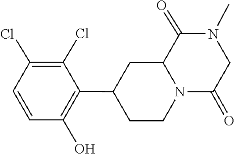 [SMILES: CN1CC(=O)N2CCC(c3c(O)ccc(Cl)c3Cl)CC2C1=O] | <1 | >30 |
| 37 | 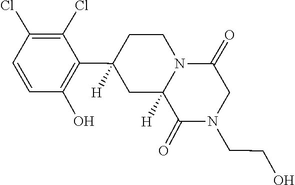 [SMILES: O=C1[C@H]2C[C@@H](c3c(O)ccc(Cl)c3Cl)CCN2C(=O)CN1CCO] | <10 | * |
| 38 | 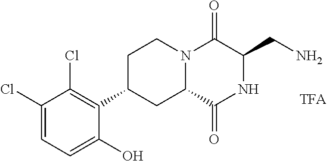 [SMILES: NC[C@H]1NC(=O)[C@@H]2C[C@H](c3c(O)ccc(Cl)c3Cl)CCN2C1=O] | <1 | * |
| 39 | 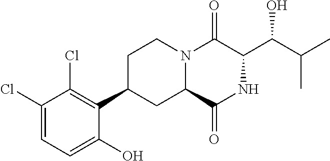 [SMILES: CC(C)[C@@H](O)[C@@H]1NC(=O)[C@H]2C[C@@H](c3c(O)ccc(Cl)c3Cl)CCN2C1=O] | <10 | * |
| 40 | 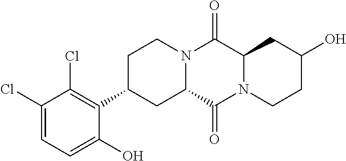 | <1 | * |
| 41 | 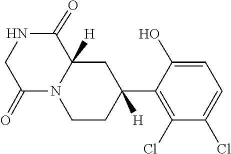 [SMILES: O=C1NCC(=O)N2CC[C@@H](c3c(O)ccc(Cl)c3Cl)C[C@@H]12] | <1 | >30 |
| 42 | 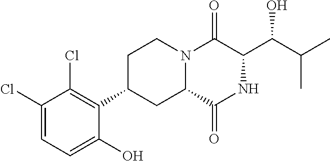 [SMILES: CC(C)[C@@H](O)[C@@H]1NC(=O)[C@@H]2C[C@H](c3c(O)ccc(Cl)c3Cl)CCN2C1=O] | <1 | * |
| 43 | 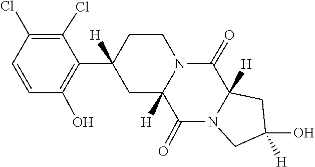 [SMILES: O=C1[C@@H]2C[C@@H](O)CN2C(=O)[C@@H]2C[C@H](c3c(O)ccc(Cl)c3Cl)CCN12] | <10 | * |
| 44 | 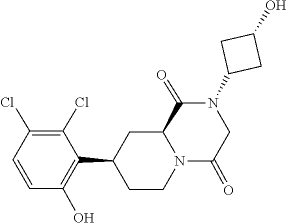 | <1 | >30 |
| 45 | 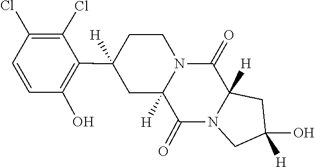 [SMILES: O=C1[C@@H]2C[C@H](O)CN2C(=O)[C@H]2C[C@@H](c3c(O)ccc(Cl)c3Cl)CCN12] | <10 | * |
| 46 | 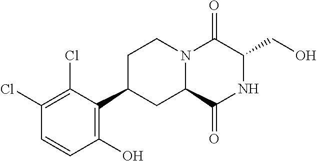 [SMILES: O=C1N[C@@H](CO)C(=O)N2CC[C@H](c3c(O)ccc(Cl)c3Cl)C[C@H]12] | <10 | * |
| 47 | 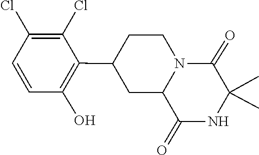 [SMILES: CC1(C)NC(=O)C2CC(c3c(O)ccc(Cl)c3Cl)CCN2C1=O] | <10 | * |
| 48 | 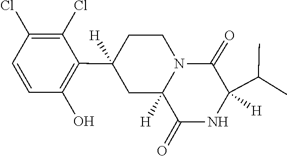 [SMILES: CC(C)[C@H]1NC(=O)[C@H]2C[C@@H](c3c(O)ccc(Cl)c3Cl)CCN2C1=O] | <10 | * |
| 49 | 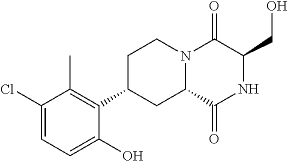 [SMILES: Cc1c(Cl)ccc(O)c1[C@@H]1CCN2C(=O)[C@@H](CO)NC(=O)[C@@H]2C1] | <10 | * |
| 50 | 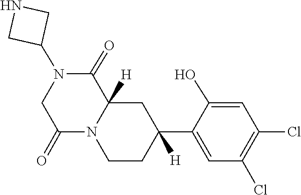 [SMILES: O=C1[C@@H]2C[C@H](c3cc(Cl)c(Cl)cc3O)CCN2C(=O)CN1C1CNC1] | <1 | >30 |
| 51 | 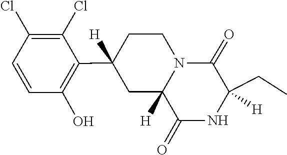 [SMILES: CC[C@H]1NC(=O)[C@@H]2C[C@H](c3c(O)ccc(Cl)c3Cl)CCN2C1=O] | <30 | * |
| 52 | 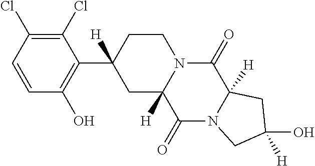 [SMILES: O=C1[C@@H]2C[C@H](c3c(O)ccc(Cl)c3Cl)CCN2C(=O)[C@H]2C[C@@H](O)CN12] | <1 | >30 |
| 53 | 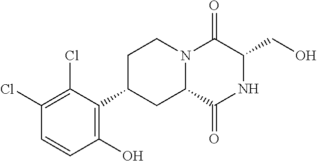 [SMILES: O=C1N[C@@H](CO)C(=O)N2CC[C@@H](c3c(O)ccc(Cl)c3Cl)C[C@@H]12] | <1 | >30 |
| 54 | 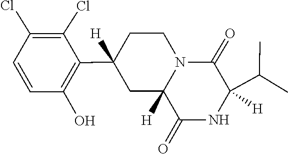 [SMILES: CC(C)[C@H]1NC(=O)[C@@H]2C[C@H](c3c(O)ccc(Cl)c3Cl)CCN2C1=O] | <1 | * |
| 55 | 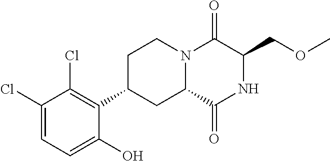 [SMILES: COC[C@H]1NC(=O)[C@@H]2C[C@H](c3c(O)ccc(Cl)c3Cl)CCN2C1=O] | <1 | >30 |
| 56 | 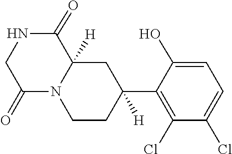 [SMILES: O=C1NCC(=O)N2CC[C@H](c3c(O)ccc(Cl)c3Cl)C[C@H]12] | <10 | * |
| 57 | 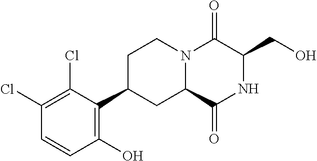 [SMILES: O=C1N[C@H](CO)C(=O)N2CC[C@H](c3c(O)ccc(Cl)c3Cl)C[C@H]12] | <10 | * |
| 58 | 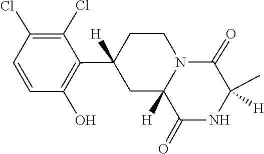 [SMILES: C[C@H]1NC(=O)[C@@H]2C[C@H](c3c(O)ccc(Cl)c3Cl)CCN2C1=O] | <1 | >30 |
| 59 | 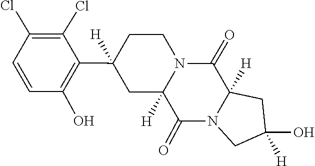 [SMILES: O=C1[C@H]2C[C@@H](O)CN2C(=O)[C@H]2C[C@@H](c3c(O)ccc(Cl)c3Cl)CCN12] | <10 | * |
| 60 | 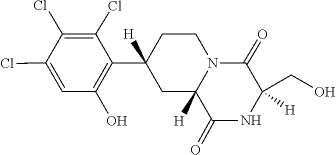 [SMILES: O=C1N[C@H](CO)C(=O)N2CC[C@@H](c3c(O)cc(Cl)c(Cl)c3Cl)C[C@@H]12] | <1 | >30 |
| 61 | 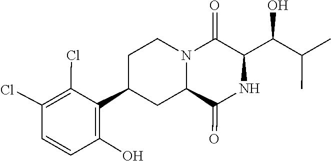 [SMILES: CC(C)[C@H](O)[C@H]1NC(=O)[C@H]2C[C@@H](c3c(O)ccc(Cl)c3Cl)CCN2C1=O] | <10 | * |
| 62 | 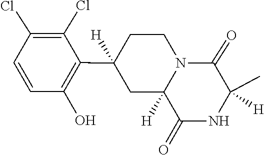 [SMILES: C[C@H]1NC(=O)[C@H]2C[C@@H](c3c(O)ccc(Cl)c3Cl)CCN2C1=O] | <10 | * |
| 63 | 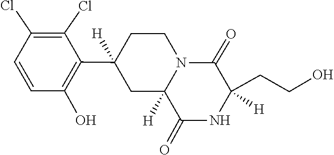 [SMILES: O=C1N[C@H](CCO)C(=O)N2CC[C@H](c3c(O)ccc(Cl)c3Cl)C[C@H]12] | <30 | * |
| 64 | 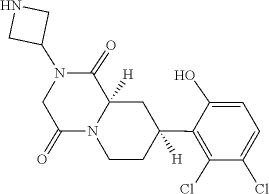 [SMILES: O=C1[C@H]2C[C@@H](c3c(O)ccc(Cl)c3Cl)CCN2C(=O)CN1C1CNC1] | <1 | >30 |
| 65 | 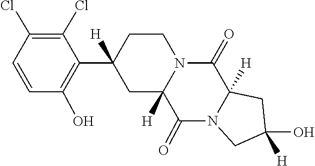 [SMILES: O=C1[C@@H]2C[C@H](c3c(O)ccc(Cl)c3Cl)CCN2C(=O)[C@H]2C[C@H](O)CN12] | <1 | <30 |
| 66 | 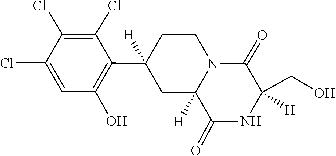 [SMILES: O=C1N[C@H](CO)C(=O)N2CC[C@H](c3c(O)cc(Cl)c(Cl)c3Cl)C[C@H]12] | <10 | * |
| 67 | 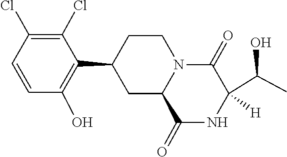 [SMILES: C[C@H](O)[C@H]1NC(=O)[C@H]2C[C@@H](c3c(O)ccc(Cl)c3Cl)CCN2C1=O] | <10 | * |
| 68 | 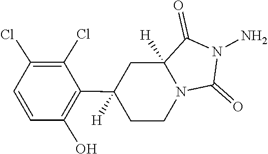 [SMILES: NN1C(=O)[C@@H]2C[C@H](c3c(O)ccc(Cl)c3Cl)CCN2C1=O] | <1 | >30 |
| 69 | 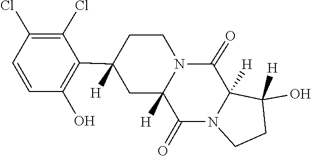 [SMILES: O=C1[C@H]2[C@H](O)CCN2C(=O)[C@@H]2C[C@H](c3c(O)ccc(Cl)c3Cl)CCN12] | <1 | * |
| 70 | 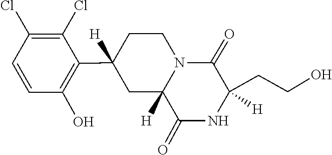 [SMILES: O=C1N[C@H](CCO)C(=O)N2CC[C@@H](c3c(O)ccc(Cl)c3Cl)C[C@@H]12] | <1 | * |

*Not Tested.

1. A compound of Formula I or a pharmaceutically acceptable salt thereof, 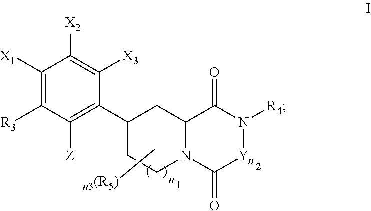
wherein
each occurrence of Y is independently C(R₂)₂ or NR₁;
Z is ORₐ;
X₁ is H, halogen, or alkyl;
X₂ is H, halogen, CN, alkyl, cycloalkyl, halogenated cycloalkyl, or halogenated alkyl;
X₃ is H, halogen, CN, alkyl, cycloalkyl, halogenated cycloalkyl, or halogenated alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is H, alkyl, cycloalkyl, heteroalkyl, or cycloheteroalkyl;
each occurrence of R₂ is H, alkyl, cycloalkyl, heteroalkyl, cycloheteroalkyl, or NRₐR_{b};
R₃ is H, alkyl, or halogen;
R₄ is H, alkyl, cycloalkyl, heteroalkyl, cycloheteroalkyl, or NRₐR_{b};
each occurrence of R₅ is H, halogen, OR₆, or alkyl, wherein each R₅ may be attached to any one of the carbon ring atoms of 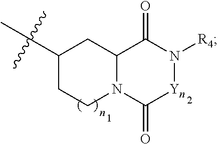
or alternatively R₁ and R₄ and the nitrogen atoms they are connected to taken together form an optionally substituted heterocycle;
or alternatively R₂ and R₄ and the carbon and nitrogen atoms they are connected to, respectively, taken together form an optionally substituted heterocycle;
each occurrence of Rₐ and R_{b} are independently H, alkyl, alkenyl, cycloalkyl, saturated heterocycle, aryl, or heteroaryl; or alternatively Rₐ and R_{b} together with the nitrogen atom that they are connected to form an optionally substituted heterocycle comprising the nitrogen atom and 0-3 additional heteroatoms each selected from the group consisting of N, O, and S;
the alkyl, cycloalkyl, heteroalkyl, cycloheteroalkyl, heterocycle, aryl, and heteroaryl in X₁, X₂, X₃, R₁, R₂, R₃, R₄, R₅, Rₐ, or R_{b}, where applicable, are each independently and optionally substituted by 1-4 substituents each independently selected from the group consisting of alkyl, cycloalkyl, halogenated alkyl, halogenated cycloalkyl, halogen, CN, OR₆, —(CH₂)₁₋₂OR₆, N(R₆)₂, (C═O)R₆, (C═O)N(R₆)₂, NR₆(C═O)R₆, and oxo where valence permits;
each occurrence of R₆ is independently H, alkyl, or heterocycle optionally substituted by alkyl; or alternatively two R₆ groups together with the nitrogen atom that they are connected to form a heterocycle optionally substituted by alkyl and comprising the nitrogen atom and 0-3 additional heteroatoms each selected from the group consisting of N, O, and S;
n₁ is an integer from 0-1;
n₂ is an integer from 0-2; and
n₃ is an integer from 0-2.

2. The compound of claim 1, wherein the structural moiety 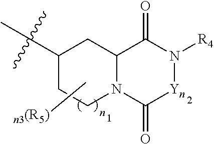
has the structure of

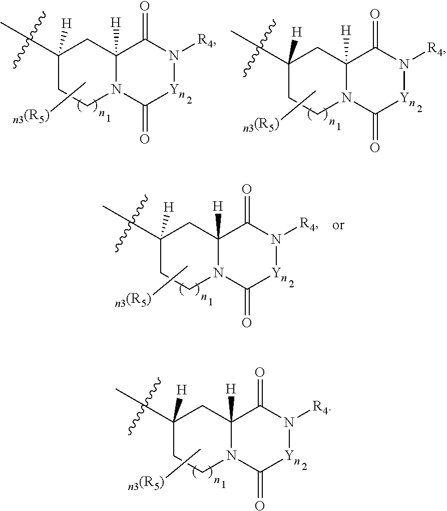

3. The compound of claim 1, wherein the structural moiety 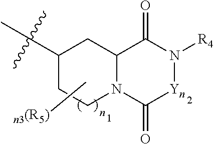
has the structure of

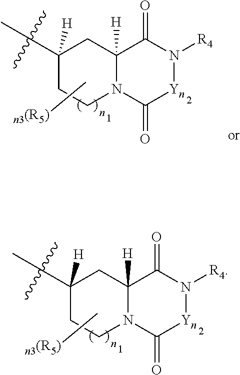

4. The compound of claim 1, wherein the structural moiety 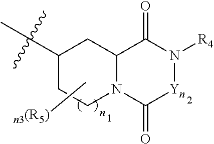
has the structure of

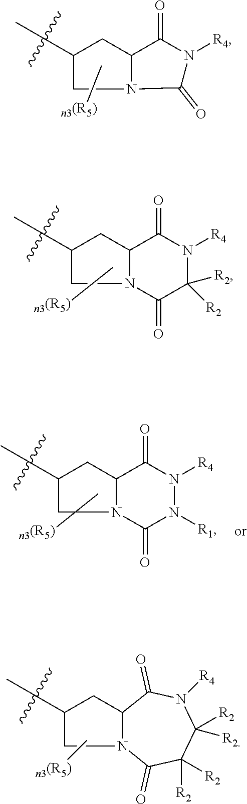

5. The compound of claim 1, wherein the structural moiety 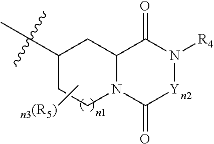
has the structure of

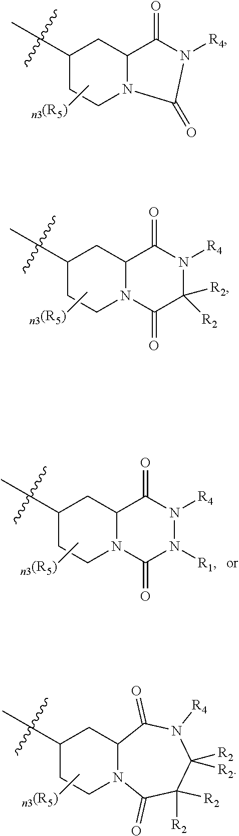

6. The compound of claim 1, wherein the structural moiety 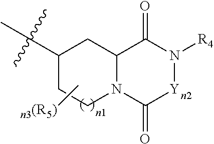
has the structure of

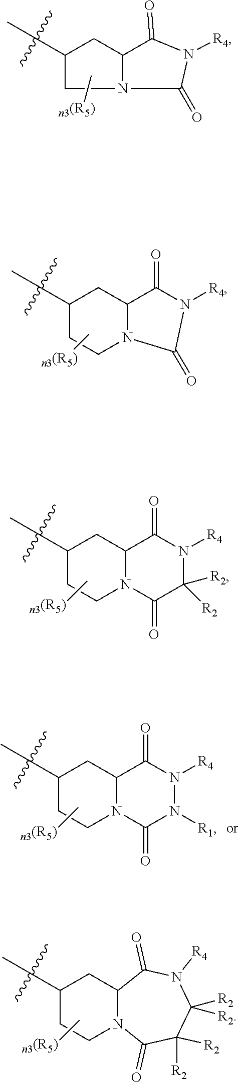

7. The compound of claim 6, wherein the structural moiety 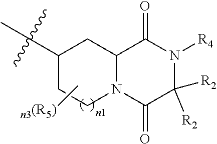
has the structure of

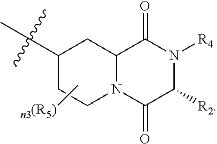

8. The compound of claim 1, wherein at least one of R₁, R₂, and R₄ is H, alkyl, or cycloalkyl.

9. The compound of claim 8, wherein at least one of R₁, R₂, and R₄ is H, Me, Et, n-Pr, iso-Pr, n-Bu, sec-Bu, or tert-Bu.

10. The compound of claim 8, wherein at least one of R₂ and R₄ is alkyl or cycloalkyl each optionally substituted by one or more OR₆, N(R₆)₂, or —(CH₂)₁₋₂OR₆.

11. The compound of claim 10, wherein at least one of R₂ and R₄ is

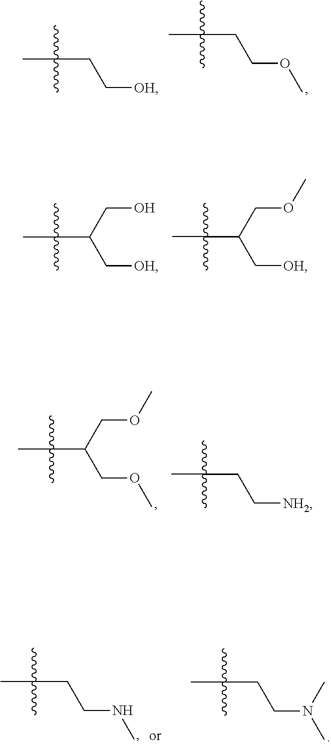

12. The compound of claim 10, wherein at least one occurrence of R₂ is Me, Et,

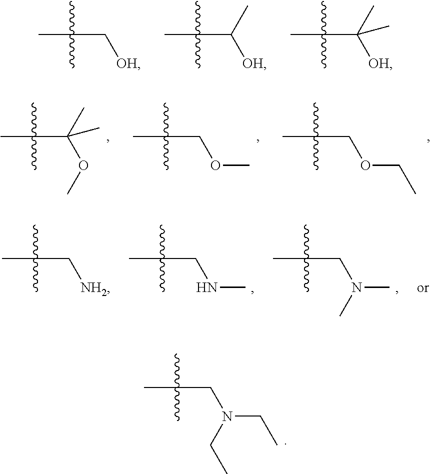

13. The compound of claim 10, wherein at least one of R₂ and R₄ is

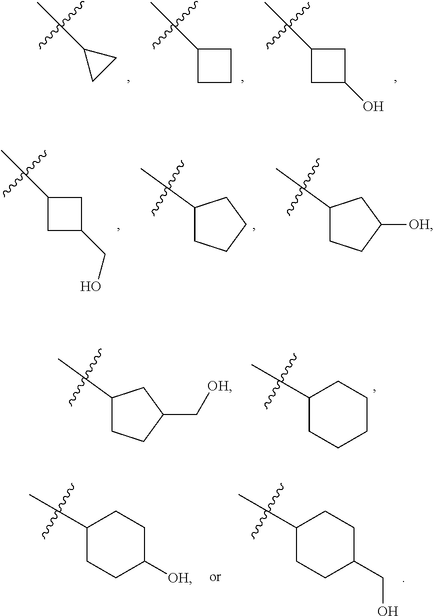

14. The compound of claim 1, wherein at least one of R₁, R₂, and R₄ is heteroalkyl, or cycloheteroalkyl.

15. The compound of claim 1, wherein at least one of R₂ and R₄ is NRₐR_{b}.

16. The compound of claim 15, wherein Rₐ and R_{b} are each independently H, alkyl or cycloalkyl.

17. The compound of claim 15, wherein at least one of R₂ and R₄ is NH₂, NHMe, or NHMe₂.

18. The compound of claim 14, wherein at least one of R₂ and R₄ is cycloheteroalkyl optionally substituted by one or more alkyl.

19. The compound of claim 18, wherein at least one of R₂ and R₄ is

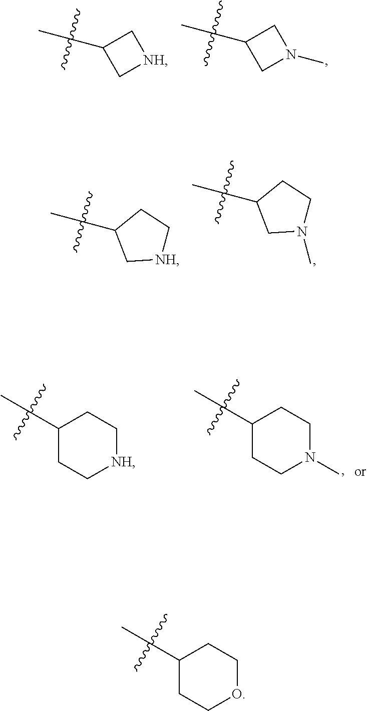

20. The compound of claim 1, wherein R₁ and R₄ and the nitrogen atoms they are connected to taken together form an optionally substituted heterocycle; or wherein R₂ and R₄ and the carbon and nitrogen atoms they are connected to, respectively, taken together form an optionally substituted heterocycle.

21. The compound of claim 20, wherein the structural moiety 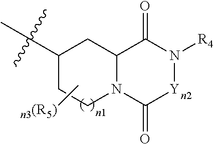
has the structure of

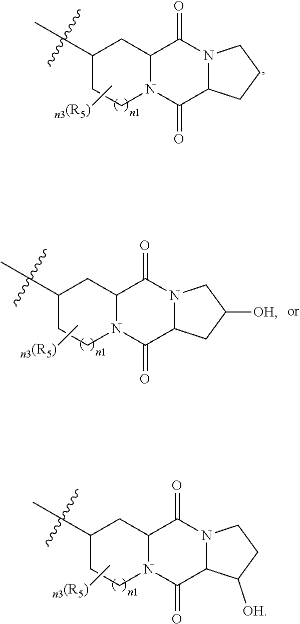

22. The compound of claim 20, wherein the structural moiety 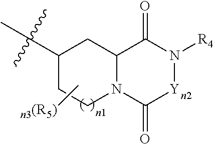
has the structure of

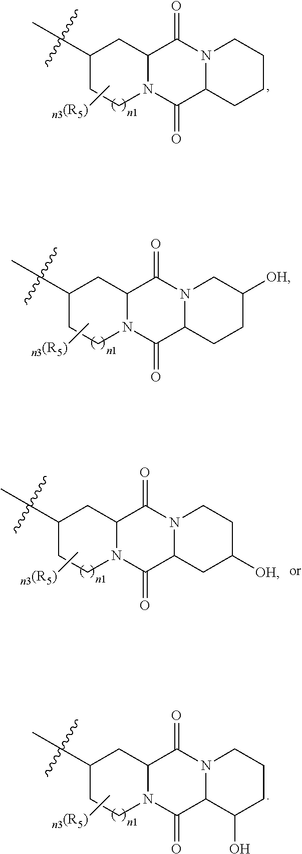

23. The compound of claim 1, wherein at least one occurrence of R₅ is H or alkyl.

24. The compound of claim 1, wherein at least one occurrence of R₅ is halogen or OH.

25. The compound of claim 1, wherein n₃ is 0 or 1.

26. The compound of claim 1, wherein Z is OH, OMe, OEt, OPr, or OBu.

27. The compound of claim 26, wherein Z is OH or OMe.

28. The compound of claim 27, wherein Z is OH.

29. The compound of claim 1, wherein X₁ is H, halogen, or Me.

30. The compound of claim 29, wherein X₁ is H or Cl.

31. The compound of claim 1, wherein X₂ is H, halogen, fluorinated alkyl, or alkyl.

32. The compound of claim 31, wherein X₂ is H, F, Cl, Br, Me, CF₂H, CF₂Cl, or CF₃.

33. The compound of claim 32, wherein X₂ is H or Cl.

34. The compound of claim 1, wherein X₃ is H, halogen, fluorinated alkyl, or alkyl.

35. The compound of claim 34, wherein X₃ is H, F, Cl, Br, Me, CF₂H, CF₂Cl, or CF₃.

36. The compound of claim 35, wherein X₃ is H or Cl.

37. The compound of claim 1, wherein R₃ is H, Me, Et, Pr, F, Cl, or Br.

38. The compound of claim 1, wherein R₃ is H.

39. The compound of claim 1, wherein R₃ is Me, Et, or Pr.

40. The compound of claim 1, wherein R₃ is F, Cl, or Br.

41. The compound of claim 1, wherein the structural moiety 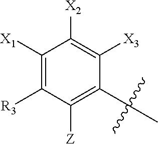
has the structure of

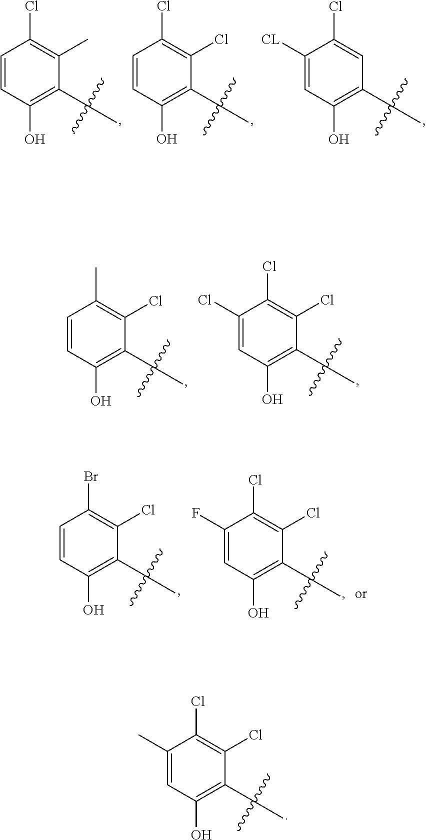

42. The compound of claim 1, wherein the compound has a structure of Formula II′ or II: 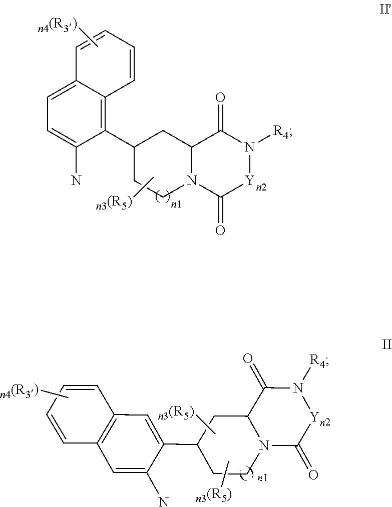
wherein R_{3′} is independently H, halogen, or alkyl; and
n₄ is an integer from 0-3.

43. The compound of claim 42, wherein n₄ is 0, 1, or 2.

44. The compound of claim 43, wherein n₄ is 0.

45. The compound of claim 42, wherein R_{3′} is H or alkyl.

46. The compound of claim 42, wherein R_{3′} is halogen.

47. The compound of claim 1, wherein at least one occurrence of Rₐ or R_{b} is independently H, alkyl, cycloalkyl, saturated heterocycle, aryl, or heteroaryl.

48. The compound of claim 47, wherein at least one occurrence of Rₐ or R_{b} is independently H, Me, Et, Pr, or a heterocycle selected from the group consisting of 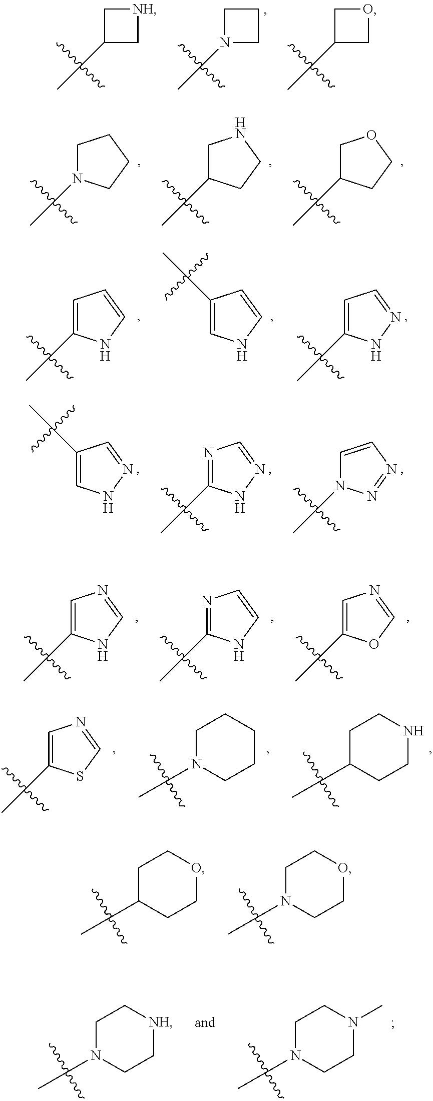
wherein the heterocycle is optionally substituted by alkyl, OH, oxo, or (C═O)C₁₋₄alkyl where valence permits.

49. The compound of claim 1, wherein Rₐ and R_{b} together with the nitrogen atom that they are connected to form an optionally substituted heterocycle comprising the nitrogen atom and 0-3 additional heteroatoms each selected from the group consisting of N, O, and S.

50. The compound of claim 1, wherein the compound is selected from the group consisting of compounds 1-70 as shown in Table 1.

51. A pharmaceutical composition comprising at least one compound according to claim 1 or a pharmaceutically acceptable salt thereof and a pharmaceutically acceptable carrier or diluent.

52. A method of treating a condition in a mammalian species in need thereof, comprising administering to the mammalian species a therapeutically effective amount of at least one compound according to claim 1 or a pharmaceutically acceptable salt thereof, wherein the condition is selected from the group consisting of cancer, an immunological disorder, a central nervous system disorder, an inflammatory disorder, a gastroenterological disorder, a metabolic disorder, a cardiovascular disorder, and a kidney disease.

53. The method of claim 52, wherein the immunological disorder is transplant rejection or an autoimmune disease.

54. The method of claim 53, wherein the autoimmune disease is rheumatoid arthritis, multiple sclerosis, systemic lupus erythematosus, or type I diabetes mellitus.

55. The method of claim 52, wherein the central nervous system disorder is Alzheimer's disease.

56. The method of claim 52, wherein the inflammatory disorder is an inflammatory skin condition, arthritis, psoriasis, spondylitis, parodontitits, or an inflammatory neuropathy.

57. The method of claim 52, wherein the gastroenterological disorder is an inflammatory bowel disease.

58. The method of claim 52, wherein the metabolic disorder is obesity or type II diabetes mellitus.

59. The method of claim 52, wherein the cardiovascular disorder is an ischemic stroke.

60. The method of claim 52, wherein the kidney disease is chronic kidney disease, nephritis, or chronic renal failure.

61. The method of claim 52, wherein the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

62. The method of claim 52, wherein the mammalian species is human.

63. A method of blocking Kv1.3 potassium channel in a mammalian species in need thereof, comprising administering to the mammalian species a therapeutically effective amount of at least one compound according to claim 1 or a pharmaceutically acceptable salt thereof.

64. The method of claim 63, wherein the mammalian species is human.
